SEQUENCE LISTING (40 sequences)

SEQ ID NO: 1 - DNA, 5520 bp, Fragaria X ananassa
  source 1..5520: mol_type = genomic DNA
aaagaagaagttgtgggcaggaatgatgtggctgccagaatggacttttctttggaaaaggcaatggggatcagatcttcacccaccacatgatacgataaatctatttctcactcaaaagcaagtctctcatcaccactttgcttctactcattcttgatctcttttgttaatagaacgaatgatttgatcaaaaactgctacatttgaagtcgatccgatccaacttcaaattcccaggaaatccaggttaagctccaactgatttctatccgttcttgcgtattacaatgttgccatgtttttgttctctgttatctttttggttatctttttctctggttagtatatctgcagctctgctcattatttattttaaaagaggcatcaattcgtaattgatcatagatttaaagtcagaatttcttatcagtttttggaacatttatgtatacttgggcactgtttagttttaattttttaaatattaacttgtggtcagataggatatatgcacttgcagctgctgctattgtttaacttcaactgtctatcaacaattcacctatcactctgtcatggggtttcagccagaactaagaaaaagaaaaatttgcttaactatctttcaatgaatcattctgacaccaattgtgttctgaagtatatttctttctagtttacaaggagctagtgttttgtgtctgtgtatttagataccataataagagaaagcaaaggagtaataatcgaccagatgaccacagaaaccaaaccagctaatgtttgttgtttcatagtcaatacagttctttttagaatgattacttattttcagttgaatgatgaggaaaattattgatcttttctatgttttatgcagtggagaagagaaaccaggacacacaaatacatggtttattggttttcatgatagttaattccatgccttgaaatagctagtattgaaatcttcccgggttactcagttggacacttactcttctacaagtaggcaagtaggtgtcaatggaaatgagcattcaaagaacctctacatctttctccccttcaaatcctcagtggaaatatgatgtctttctgagctttagaggtgatgacacccgcaagactttcacagaccacttatacactgcattggaacatcaaggaatcataactttcagggatgatcctgaacttaaaaaagggaaagctatttctccagaacttttttctgcaattaaagaatcaagatttgctctcattattctgtcacaaaattatgcatcgtcaacatggtgcttggatgaacttgtaagagttcttgaatgcatgaaagcaagagaaactgtgctgccaattttctatgatattgatccctctgatgtacgaaagcaaactggtagttttggagaagccttcactcatcacgaagaaaggtttagggatgacaaagagaaagtgcgaaggtggagagatgctttaactgaagtggcaagtttctctgggtggaattcaaaggaatggtatgcatatactcttttatttttgtctcatcaatctacagtggttgattttggtgctcttcattatttttttcacaaataatctagtcagatagtcaatctgatagtttatgaggcttttttgtcattactgaaatataaaacaaattgcagatacattttcattaactagatcttcatatcaataacttgatacttttattcttttatatgtaataggtatgaatcaaagctcattacaactattgttgaagttatatggacaaaattgcaacctacatcattcagttatgcagaaaatctagttgggattgattcaagattgcagcaagtcaatttgcttttaggtgtaggggtggatgatgtccgcttcattgggatatttggaatgggcgggattggtaagacaactatggtaagagcggtgtatgagagaatcgctcgtgaatttgaatttagtttccttctcagcgatgttagaaactctgttgagaaaagtggtctacttaatttacaaaagcaacttctctcggggatttggagaaagaaggcagagataacggaccttcatggaggagccaccattataaggagattgttaggtcacaaaaaagttcttctcattcttgatgatgtgaaccattcaagccatttaaaatatttggcaggaaaccaagagtggtttggttcagggagtagagttctaatcacaactagaaatgagcatttgttgattgaacatggagtggagagaagattgatggtggaagaattaaatgatgatgattctcttcagctctttagctggaaagcgttcaaaagaggttacactgaagaagattttcttggtttgtccaaatctgttgtaagttatgccaaaggtcttcctttagctcttgaagtactgggttcttttttgcatggaagagatgtaactgaatggaagagtgcactgagaaaactgggaagagtctgtaacttggaaatttttgatgtgctaaaaataagttataaggatctagattctgaagagaagaaaatattcctagacattgcatgtttctttaatggacaggacaaagatcgagttactgaagtattaaacagttgcaatgtctctgcaattattggaataaaagtcctgatggaaagatctttgttgactgtttcatatgggagacttaggatgcacgatttgctccaagaaatgggacgggaaattgtccgctcggaatatcctaacgagccgggcaggtgcagtaggttgtggcttgttgaagacatcaaaaatgtcttaaccaataataccgtaagagatttagaagaagaatcatttgaattgttattttctaatatattatatgaccagtcagtctactaaatctgctttttattgtatgatgacaatgggttaatagggaactgaagcaatagaaggcatatttgtggactccactgagtcggtagcagaggtggacgtgactccgaaatcattttcaatgatgaacaaattgagatacctgaagattaaaaatgggaacctacctaaggggctggaatatcttcccaatagtttactgattcttgattggactaggtatccgtcaaagtctctaccatcacatttcaaccctcagaagctgctggaacttagcttgtgtcacagctctattaaacatgtccggataggaacagaggtatactattattactattttaacgtataatgattgttgattatatcatggaagctaattttttctatgctttttacttgacgtgcagcctttatacaatttgaaaactgttgatctgagttactctctggatcttgtcaacaccccaaactttaaaagtatgccacatctagaggttttgtttcttgaaggttgtacaagattgtatgaggttgacccaacaattgaagtgcttgaaagacttactgtgcttaacttgaaagattgcaagaatctggtgcattttgcaagcagtgtaagtggcttaaaatctctcaaagttcttaatctttctggttgttcaaaaattaacaaactaccacatgacacgggtcatttggcgagcttggaggagctttatctgaatgacactggcataagggaactgcctacgtctactggaatgcttgaaagactttctctgctaaacatggaagattgcaaacattttgcgtgtcttcccatcagtgtaggtggtttaaaatctctgaaagatctcaatctgtctggttgctcaaagcttgacaaattgccagacgagctgagtcatgtttcctgtttagagaagcttgatgtgagtggaagtggcataagagaagtgccctcctttatcggtcttttgaaaaacctcaaaagattatctcttgctggatgtaaagcagtgtcacctaaatcatggagtataatgttcaacccctttcagttattgcaaaaaagagcttgcgttcccgcaggattgtcactggcttgtttgtctggtttgcattctttaaccaaactggatctaagtgactgtaatctttctgaagaagcaatccccagtgattttggatgctcgttagcctcattgaagatattacatttgagcaacaatcaatttattagactaccagagagcatcggccaactctctagacttgaatctctttacttgaaagggtgccgcaagcttcagacattaccagagtttcgatctgatgtacatgtagacgctctcaactgcacttcattagattcattggtcaatcagataggacaacgcaattcggtgctacgagcgagatgtgttaactgttttaaaatggtggagaataatgatagctataagagcagtgcaatttcattgctaagacgctaccttaagtttcgacgtcagagttcttctgtagattcaaagcataaaacatttgaattcattgctccagggaatgaaattccagagtggtacaaccaccaaagcgtggggccttttataactctagagctgcatcctggttggtttactaacaagtttatgggattcgccacctgtgtcagttttagaatggtgaaaccactcccgcctgcagtcgatgacttttcgatcagtatgctcttgaatattaatggagcatacaaagtgcatggctttgctataaattttgagtttggggaaaagtgcgatcaacctaagttggatcacatatggtttttctatatgcaccgtgatacatactcttattttgagtggcaggacatctactatcagcttgtattcacagtccactgctcggtgccaacagaaagccgcctaacaggtcatgaatgggagtttgcagggatggatgaggtgcaagtgaagaagtgtggtgtgcgtctggtctatgaggaagatgtggaggagcagagcaattttggctttaataccaagcgaggccttcaacacgaagatgaagaagaggatgaagcatctagtgcaagtcaggctcatccaaaaagaaccaaacaactccatcttgacggcagttgacccggtggaatggaagcccttatattaagtacgacggaaaggttgtccatatgggtggcaagtggcaacaacttctgtcttgtgttgtatggttttattgcaggccgaccgttgtggtcttcgaaagtagcaacatttcaagtttaatattgttggaggagggcaatggttctaccatgggaattcgctgcatttaccatcactttaacaatattaagccacggtttagtaatccaactccgcctcggatgaggtacccgatttcaaggggttatgaattagatcagtacggagatttccaggatgctttcgtctaaaaaaagttagttctgattcagtaattcatttgtttattaattacttgatagaaattggtttgccaactatcgaaaattttcagttaacgaagcacccctatattcccagaaaatttaactcatctgctattctcttctttcttgatccc

SEQ ID NO: 2 - protein, 1202 aa, Fragaria X ananassa
MEMSIQRTSTSFSPSNPQWKYDVFLSFRGDDTRKTFTDHLYTALEHQGIITFRDDPELKKGKAISPELFSAIKESRFALIILSQNYASSTWCLDELVRVLECMKARETVLPIFYDIDPSDVRKQTGSFGEAFTHHEERFRDDKEKVRRWRDALTEVASFSGWNSKEWYESKLITTIVEVIWTKLQPTSFSYAENLVGIDSRLQQVNLLLGVGVDDVRFIGIFGMGGIGKTTMVRAVYERIAREFEFSFLLSDVRNSVEKSGLLNLQKQLLSGIWRKKAEITDLHGGATIIRRLLGHKKVLLILDDVNHSSHLKYLAGNQEWFGSGSRVLITTRNEHLLIEHGVERRLMVEELNDDDSLQLFSWKAFKRGYTEEDFLGLSKSVVSYAKGLPLALEVLGSFLHGRDVTEWKSALRKLGRVCNLEIFDVLKISYKDLDSEEKKIFLDIACFFNGQDKDRVTEVLNSCNVSAIIGIKVLMERSLLTVSYGRLRMHDLLQEMGREIVRSEYPNEPGRCSRLWLVEDIKNVLTNNTGTEAIEGIFVDSTESVAEVDVTPKSFSMMNKLRYLKIKNGNLPKGLEYLPNSLLILDWTRYPSKSLPSHFNPQKLLELSLCHSSIKHVRIGTEPLYNLKTVDLSYSLDLVNTPNFKSMPHLEVLFLEGCTRLYEVDPTIEVLERLTVLNLKDCKNLVHFASSVSGLKSLKVLNLSGCSKINKLPHDTGHLASLEELYLNDTGIRELPTSTGMLERLSLLNMEDCKHFACLPISVGGLKSLKDLNLSGCSKLDKLPDELSHVSCLEKLDVSGSGIREVPSFIGLLKNLKRLSLAGCKAVSPKSWSIMFNPFQLLQKRACVPAGLSLACLSGLHSLTKLDLSDCNLSEEAIPSDFGCSLASLKILHLSNNQFIRLPESIGQLSRLESLYLKGCRKLQTLPEFRSDVHVDALNCTSLDSLVNQIGQRNSVLRARCVNCFKMVENNDSYKSSAISLLRRYLKFRRQSSSVDSKHKTFEFIAPGNEIPEWYNHQSVGPFITLELHPGWFTNKFMGFATCVSFRMVKPLPPAVDDFSISMLLNINGAYKVHGFAINFEFGEKCDQPKLDHIWFFYMHRDTYSYFEWQDIYYQLVFTVHCSVPTESRLTGHEWEFAGMDEVQVKKCGVRLVYEEDVEEQSNFGFNTKRGLQHEDEEEDEASSASQAHPKRTKQLHLDGS

SEQ ID NO: 3 - DNA, 3656 bp, Fragaria X ananassa
  source 1..3656: mol_type = genomic DNA
gaataccatattccccatgcttccaagctccaacctttgcgtctccaaattctcttcctttctccctctccactatgaacacgtgaacaccacctctctcaccaatgtgaagtcatatccaacttccaagccacctttccggtaccattccaacttcaccgtaaccaaaaaacgggcgccacaaccttaaagcataacccccttcaccggacggcatgttgttcctccggcaacggcaactccctccagttctccttctctcgctcctcttcctctcctcactctctccctccacatcaaacgagctcgaccaactcttgaagatcaaagcctcacttgaaacttcaaacccaactctgttcagttcatggaaccaagaaactcccacgtgcaacttcactgggattgtctgcaactcaaatgagctcgtctcagaaatcaatctttctcagcagaacctctctggctctcttcctttcgacgcattatgttcccttcctgaactcaaaaagctctcttttggttcaaattttctacacggaagcctgacagaggatttgaaaaagtgtactaatttgcaggaacttgatttgggtaagaattctttcactggagaagtgcctgagttgggttctttgagtcagttgaggctgctgagtctgaatgctagtggggtttcagggctttttccatggaagtcacttgggaatctgacgaagttggcttttttgagccttggtgataatgtattcgatcaaagtccgattacaatggagttgtcaaagcttgacaagctttactggctttacctctcaaactgtagcatcacaggtgaaatcccaaatggtattggaaacctgactctgcttcagaatctggagcttgctgataaccaattggttggtgaaattcctcagagtattataaatctcaacaagctttggcagcttgagctttatggtaattctctgagtgggaaattcccagttgggtttggcaacctcacaagcctcaggaactttgatgcttcaaaaaacaagcttgagggtgatctctctgagctcaagtccttgacagggcttgtgtctctgcagctgcttgagaatcaattagtcggcgaagtcccggaagagtttggggagttcaagaaccttgttgagctctccctttacaccaacaagcttactggctctcttcctcagaagcttggttcttggtctggcatggagtatattgatgtttcagagaactatttgactggtcctataccccctgggatgtgcaagaatgatagaatgattgatattcttctgctgcagaacaatttcacaggtgggattcctgaatcctatgcagattgcaagtctttggtccggttacgtgtgaataacaattcactttcgggtactgtccctgctgggatttggagcttgccaaatgttactattcttgatctctctatgaatcaatttgaaggtcccctggcttcagatgttggtaaggctaattctctgtcattattactgttaaataataatcgattttggggtgaactgcctgctgctttgtctgaagctacatcattggttacaattcagcttagtgagaaccaatttgagggtcaaattccatcaacaattgggaacttgaagaaattaggtagtcttcatttagagcataatatgttgtctggtgccataccagactcactaggttcatgtgttaggatttctgaaatcggccttgcccataacattctctctggccaaatcccaccaagtcttggatcattgcctaacttgaactctttgaacttatctggaaaccagctttctagtgaagttcctacaactttgtcatctctcaaactcagccttcttgatcttacaaacaaccagttgtctggccgcgtgcccgagtcactatccattgcagcttttaatgaaagctttgctggaaatcctggtttgtgcagccaaaccatgaagaatttgcggccttgttcttcaaattcagactctcctagtcacccaaggatatttttgtcttcttttattgctggagtacttcttctgcttattttgcttggtgtgttcttgctatggaagctaaggaggagcgatcttgatcaaccattgaagagtaattcatggactatgaagcagtaccatgtgttaagcttcacagagaatgaaatcctagagtcaatcaaagctgaaaacttgattggcaagggagggtcaggaaatgtgtacaaagtacaactgagagatgggaaagcacttgctgtgaagcacatctggacctcagcagactctgctaaccagaaaacttatcgaagcactacttccattttgaagaagcctaaaacaagctcatcggagtatgatgccgaggtggccacactcagctcattgaggcatgtgaatgtggtgaagctatactgcagcataagtagtgaggacagcaagctgctggtttacgagtactttcctaatgggagcttatgggatcagctgcacatgaataagaaggccaagatggggtgggaagtgaggtatgagattgctttgggggctgcaagaggattggagtatctccaccatggtagtcacagacctgttatacatagagatgtgaagtctagtaatattctgctagatggagattggaagcctagaattgctgattttgggcttgccaagattgtgcaggctggtggtgaaatgactcatgtcattgctgggacacatggctacatagctcctggtaaattctaattatattactatctttgttcttatttagatttgtagtttagattgatagtctcgcttagtcgcaacattacatgtcaaatgaacttatgagactgaaatgattgaacttcttgatgttttggtggttcagaatatgcatatacctacaaggtgaacgagaagagtgatgtgtatagctttggggttgtcttgatggagttggtgacaggaaagaggccagtggagcctgagtttggggagaacaaggacatagtgagctgggtttgcagtaaaataatttgcaaagatactgagcttgaattggtggactcgagcatcgaagaggctctaaaagaatatgccctcaacgttttgagcattgcaattcgctgcaccgctagagttccggttctaagaccctcaatgagaatggtggttcaaatgctggaagaggcacaaccttgtcagctcaccagcataagcatcaccaaggaaggtgaaagtaacccgaaaatggagcagaatttccactacggaaaaatgtagagtagttgcagttggtggtctgtgggtttttgctttggggtaagatggtcagatacagaaagaaagctaattgagaggaaagaagctcctgactacaaagtttgggaggaactgtatcaattagagatgtaagcagaaatacatcgtttttgttttggccgctcactgagaggggtttactttataatataataaaggtccaccgggggaggggggtgaatatgcattttttctaatttttgttaacttctatatcgaagatagtaataacacggggtgaatatgcattttttctaatttttgttaacttgtgttcgaagatagtaataacttgtgttatcccggattcac

SEQ ID NO: 4 - protein, 982 aa, Fragaria X ananassa
MLFLRQRQLPPVLLLSLLFLSSLSPSTSNELDQLLKIKASLETSNPTLFSSWNQETPTCNFTGIVCNSNELVSEINLSQQNLSGSLPFDALCSLPELKKLSFGSNFLHGSLTEDLKKCTNLQELDLGKNSFTGEVPELGSLSQLRLLSLNASGVSGLFPWKSLGNLTKLAFLSLGDNVFDQSPITMELSKLDKLYWLYLSNCSITGEIPNGIGNLTLLQNLELADNQLVGEIPQSIINLNKLWQLELYGNSLSGKFPVGFGNLTSLRNFDASKNKLEGDLSELKSLTGLVSLQLLENQLVGEVPEEFGEFKNLVELSLYTNKLTGSLPQKLGSWSGMEYIDVSENYLTGPIPPGMCKNDRMIDILLLQNNFTGGIPESYADCKSLVRLRVNNNSLSGTVPAGIWSLPNVTILDLSMNQFEGPLASDVGKANSLSLLLLNNNRFWGELPAALSEATSLVTIQLSENQFEGQIPSTIGNLKKLGSLHLEHNMLSGAIPDSLGSCVRISEIGLAHNILSGQIPPSLGSLPNLNSLNLSGNQLSSEVPTTLSSLKLSLLDLTNNQLSGRVPESLSIAAFNESFAGNPGLCSQTMKNLRPCSSNSDSPSHPRIFLSSFIAGVLLLLILLGVFLLWKLRRSDLDQPLKSNSWTMKQYHVLSFTENEILESIKAENLIGKGGSGNVYKVQLRDGKALAVKHIWTSADSANQKTYRSTTSILKKPKTSSSEYDAEVATLSSLRHVNVVKLYCSISSEDSKLLVYEYFPNGSLWDQLHMNKKAKMGWEVRYEIALGAARGLEYLHHGSHRPVIHRDVKSSNILLDGDWKPRIADFGLAKIVQAGGEMTHVIAGTHGYIAPEYAYTYKVNEKSDVYSFGVVLMELVTGKRPVEPEFGENKDIVSWVCSKIICKDTELELVDSSIEEALKEYALNVLSIAIRCTARVPVLRPSMRMVVQMLEEAQPCQLTSISITKEGESNPKMEQNFHYGKM

SEQ ID NO: 5 - protein, 85 aa, Fragaria X ananassa
SFRMVKPLPPAVDDFSISMLLNINGAYKVHGFAINFEFGEKCDQPKLDHIWFFYMHRDTYSYFEWQDIYYQLVFTVHCSVPTESR

SEQ ID NO: 6 - DNA, 3615 bp, Fragaria X ananassa
  source 1..3615: mol_type = other DNA
atggaaatgagcattcaaagaacctctacatctttctccccttcaaatcctcagtggaaatttgatgtctttctgagctttagaggtgatgacacccgcaagactttcacagaccacttatacactgcattggaacatcaaggaatcataactttcagggatgatcctgaacttaaaaaagggaaatctatttctccagaacttttttctgcaattaaagaatcaagatttgctctcattattctgtcacaaaattatgcatcgtcaacatggtgcttggatgaacttgtaagaattcttgaatgcatgaaagcaagagaaactgtgctgccaattttctatgatattgatccctctgatgtacgaaagcaaacaggtagttttggagaagccttcactcatcacgaagaaaggtttagggatgacaaacagaaagtgcgaaggtggagagatgctttaactgaagtggcaagtttttctgggtggaattcaaaggaatggtatgaatcaaagctcattacaactattgttgaagttatatggacaaaattgcaacctacatcattcagttatgcagaaaatctagttgggattgattcaagattgcagcaagtcaatttgcttttaggtgtaggggtggatgatgtccgcttcattgggatatttggaatgggcgggattggtaagacaactatggtaagagcggtgtatgagagaatcgctcgtgaatttgaatttagtttccttctcagcgatgttagaaactctgttgagaaaagtggtctacttaattttcaaaagcaacttctctcggggatttggagaaagaaggcggacataacagaccttcatggaggagccaccattataaggaggttattaggtcacaaaaaagttcttctcattcttgatgatgtgaaccattcaagccatttaaaatatttggcaggaaaccaagagtggtttggttcagggagtagagttctaatcacaactagaaatgagcatttgttgattgaacatggagtggagagaagattgatggtggaagaattaaatgatgatgattctcttcagctctttagctggaaagcgttcaaaagaggttacactgaagaagattttcttggtttgtccaaatctgttgtaagttatgccaaaggtcttcctttagctcttgaagtactgggttcctttttgcatggaagagatgcaactgaatggaagagtgcactgagaaaactgggaagagtctgtaacttggaaatttttgatgtgctaaaaataagttataatgatctagattctgaagagaagaaaatattcctagacattgcatgtttctttaatggacaggaaaaagatcgagttactgaagtattaaacagttgcaatgtctctgcaattattggaataaaagtcctgatggaaagatctttgttgactgtttcatatgggagacttaggatgcacgatttgctccaagaaatgggacgggaaattgttcgctcggaatatcctaacgagccgggcaggtgcagtaggttgtggcttgttgaagacatcaaaaatgtcttaaccaataataccggaactgaagcaataaaaggcatatttgtggactccactgagtcgggagcagaggtggacgtgactccgaaatcattttcaatgatgaacaaattgagatacctgaagattaaaaatgggaacctaccgaaggggctggaatatcttcccaatagtttactgattcttgattggactaggtatccgtcaaagtctctaccatcacatttcaaccctcagaagctgctggaacttagcttgtgtcacagctctattaaacatgtccggagaggaacagagcctttatacaatttgaaaactgttgatctgagttactctctggatcttgtcaacaccccaaactttaaaagtatgccacatctcgaggttttgtttcttgaaggttgtacaagattgtatgaggttgacccaacaattgaagtgcttgaaagacttactgtgcttaacttgaaagattgcaagaatctggtgcattttgcaagcagtgtaagtggcttaaaatctctcaaagttcttaatctttctggttgttcaaaaattaacaaactaccacatgacacgggtcatttggcgagcttggaggagctttatctgaatgacactggcataagggaactgcctacgtctactggaatgcttgaaagactttctctgctaaacatggaagattgcaaacattttgcgtgtcttcccattagtgtaggtggtttaaaatctctgaaagagctcaatctgtctggttgctcaaagcttgacaaattgccagacgagctgagtcatgttgcctgtttagagaagcttgatgtgagtggaagcggcataagagaagtgccctcctttatcggtcttttgaaaaacctcaaaagattatctcttgctggatgtaaagcagtgtcacctaaatcatggagtataatgttcaacccctttcagttattgcaaaaaagagcttgcgttcccgcaggattgtcactggcttgtttgtctggtttgcattctttaacccaactggatctaagtgactgtaatctttctgaagaagcgatccccagtgattttggatgctcattagcctcattgaagatattacatttgagcaataatcaatttattagactaccagagagcatcggccaactctctagacttgaatctctttacttgaaagggtgccgcaagcttcagacattaccagagtttcgatctgatgtacatgtagacgctctcaactgcacttcattagattcattggtcaatcagataggacaatgcaattcggtgctacgagcaagatgtgttaactgttttaaaatggtggagaataatgatagctataagagcagtgcaatttcattgctaagacgctaccttaagtttcgacgtcagagttcttctgtagattcaaagcataaaacatttgaattcattgctccagggaatgaaattccagagtggtacaaccaccaaagcgtggggccttttataactctagagctgcatcctggttggtttactaacaagtttatgggattcgccacctgtgtcatttttagaatggtgaaaccactcccgcctgcagtcgatgacttgtcgatctgtatgttcttgaatattattggagcaaacgaagtgcatggctttggtttaaactttgagtgtggggaaaagtgcgatcaacctaagttggatcacatatggtttttctatatgcatcgtgatacatactcttattttgagtggcaggacatctactatcagcttgtattctcattcccaccctactactcggtgcgaacagaaagcaacctaacaggtcatgaatgggagtttgcagggatggatgaggtgcaagtgaagaagtgtggtgtgcgtgtggtctatgaggaagatgtggaggagcagagcaattttggctttaataccaagcgaggccttcaacacgaagatgaagaagagggtgaagcatctagtagtgcaagtcaggctcatccaaaaagaaccaaacaactccatcttgacggcagt

SEQ ID NO: 7 - protein, 1205 aa, Fragaria X ananassa
MEMSIQRTSTSFSPSNPQWKFDVFLSFRGDDTRKTFTDHLYTALEHQGIITFRDDPELKKGKSISPELFSAIKESRFALIILSQNYASSTWCLDELVRILECMKARETVLPIFYDIDPSDVRKQTGSFGEAFTHHEERFRDDKQKVRRWRDALTEVASFSGWNSKEWYESKLITTIVEVIWTKLQPTSFSYAENLVGIDSRLQQVNLLLGVGVDDVRFIGIFGMGGIGKTTMVRAVYERIAREFEFSFLLSDVRNSVEKSGLLNFQKQLLSGIWRKKADITDLHGGATIIRRLLGHKKVLLILDDVNHSSHLKYLAGNQEWFGSGSRVLITTRNEHLLIEHGVERRLMVEELNDDDSLQLFSWKAFKRGYTEEDFLGLSKSVVSYAKGLPLALEVLGSFLHGRDATEWKSALRKLGRVCNLEIFDVLKISYNDLDSEEKKIFLDIACFFNGQEKDRVTEVLNSCNVSAIIGIKVLMERSLLTVSYGRLRMHDLLQEMGREIVRSEYPNEPGRCSRLWLVEDIKNVLTNNTGTEAIKGIFVDSTESGAEVDVTPKSFSMMNKLRYLKIKNGNLPKGLEYLPNSLLILDWTRYPSKSLPSHFNPQKLLELSLCHSSIKHVRRGTEPLYNLKTVDLSYSLDLVNTPNFKSMPHLEVLFLEGCTRLYEVDPTIEVLERLTVLNLKDCKNLVHFASSVSGLKSLKVLNLSGCSKINKLPHDTGHLASLEELYLNDTGIRELPTSTGMLERLSLLNMEDCKHFACLPISVGGLKSLKELNLSGCSKLDKLPDELSHVACLEKLDVSGSGIREVPSFIGLLKNLKRLSLAGCKAVSPKSWSIMFNPFQLLQKRACVPAGLSLACLSGLHSLTQLDLSDCNLSEEAIPSDFGCSLASLKILHLSNNQFIRLPESIGQLSRLESLYLKGCRKLQTLPEFRSDVHVDALNCTSLDSLVNQIGQCNSVLRARCVNCFKMVENNDSYKSSAISLLRRYLKFRRQSSSVDSKHKTFEFIAPGNEIPEWYNHQSVGPFITLELHPGWFTNKFMGFATCVIFRMVKPLPPAVDDLSICMFLNIIGANEVHGFGLNFECGEKCDQPKLDHIWFFYMHRDTYSYFEWQDIYYQLVFSFPPYYSVRTESNLTGHEWEFAGMDEVQVKKCGVRVVYEEDVEEQSNFGFNTKRGLQHEDEEEGEASSSASQAHPKRTKQLHLDGS

SEQ ID NO: 8 - DNA, 3618 bp, Fragaria X ananassa
  source 1..3618: mol_type = other DNA
atggaaatgagcattcaaagaacctctacatctttctccccttcaaatcctcagtggaaatttgatgtctttctgagctttagaggtgatgacacccgcaagactttcacagaccacttatacactgcattggaacatcaaggaatcataactttcagggatgatcctgaacttaaaaaagggaaatctatttctccagaacttttttctgcaattaaagaatcaagatttgctctcattattctgtcacaaaattatgcatcgtcaacatggtgcttggatgaacttgtaagaattcttgaatgcatgaaagcaagagaaactgtgctgccaattttctatgatattgatccctctgatgtacgaaagcaaacaggtagttttggagaagccttcactcatcacgaagaaaggtttagggatgacaaacagaaagtgcgaaggtggagagatgctttaactgaagtggcaagtttttctgggtggaattcaaaggaatggtatgaatcaaagctcattacaactattgttgaagttatatggacaaaattgcaacctacatcattcagttatgcagaaaatctagttgggattgattcaagattgcagcaagtcaatttgcttttaggtgtaggggtggatgatgtccgcttcattgggatatttggaatgggcgggattggtaagacaactatggtaagagcggtgtatgagagaatcgctcgtgaatttgaatttagtttccttctcagcgatgttagaaactctgttgagaaaagtggtctacttaattttcaaaagcaacttctctcggggatttggagaaagaaggcggacataacagaccttcatggaggagccaccattataaggaggttattaggtcacaaaaaagttcttctcattcttgatgatgtgaaccattcaagccatttaaaatatttggcaggaaaccaagagtggtttggttcagggagtagagttctaatcacaactagaaatgagcatttgttgattgaacatggagtggagagaagattgatggtggaagaattaaatgatgatgattctcttcagctctttagctggaaagcgttcaaaagaggttacactgaagaagattttcttggtttgtccaaatctgttgtaagttatgccaaaggtcttcctttagctcttgaagtactgggttcctttttgcatggaagagatgcaactgaatggaagagtgcactgagaaaactgggaagagtctgtaacttggaaatttttgatgtgctaaaaataagttataatgatctagattctgaagagaagaaaatattcctagacattgcatgtttctttaatggacaggaaaaagatcgagttactgaagtattaaacagttgcaatgtctctgcaattattggaataaaagtcctgatggaaagatctttgttgactgtttcatatgggagacttaggatgcacgatttgctccaagaaatgggacgggaaattgttcgctcggaatatcctaacgagccgggcaggtgcagtaggttgtggcttgttgaagacatcaaaaatgtcttaaccaataataccggaactgaagcaataaaaggcatatttgtggactccactgagtcgggagcagaggtggacgtgactccgaaatcattttcaatgatgaacaaattgagatacctgaagattaaaaatgggaacctaccgaaggggctggaatatcttcccaatagtttactgattcttgattggactaggtatccgtcaaagtctctaccatcacatttcaaccctcagaagctgctggaacttagcttgtgtcacagctctattaaacatgtccggagaggaacagagcctttatacaatttgaaaactgttgatctgagttactctctggatcttgtcaacaccccaaactttaaaagtatgccacatctcgaggttttgtttcttgaaggttgtacaagattgtatgaggttgacccaacaattgaagtgcttgaaagacttactgtgcttaacttgaaagattgcaagaatctggtgcattttgcaagcagtgtaagtggcttaaaatctctcaaagttcttaatctttctggttgttcaaaaattaacaaactaccacatgacacgggtcatttggcgagcttggaggagctttatctgaatgacactggcataagggaactgcctacgtctactggaatgcttgaaagactttctctgctaaacatggaagattgcaaacattttgcgtgtcttcccattagtgtaggtggtttaaaatctctgaaagagctcaatctgtctggttgctcaaagcttgacaaattgccagacgagctgagtcatgttgcctgtttagagaagcttgatgtgagtggaagcggcataagagaagtgccctcctttatcggtcttttgaaaaacctcaaaagattatctcttgctggatgtaaagcagtgtcacctaaatcatggagtataatgttcaacccctttcagttattgcaaaaaagagcttgcgttcccgcaggattgtcactggcttgtttgtctggtttgcattctttaacccaactggatctaagtgactgtaatctttctgaagaagcgatccccagtgattttggatgctcattagcctcattgaagatattacatttgagcaataatcaatttattagactaccagagagcatcggccaactctctagacttgaatctctttacttgaaagggtgccgcaagcttcagacattaccagagtttcgatctgatgtacatgtagacgctctcaactgcacttcattagattcattggtcaatcagataggacaatgcaattcggtgctacgagcaagatgtgttaactgttttaaaatggtggagaataatgatagctataagagcagtgcaatttcattgctaagacgctaccttaagtttcgacgtcagagttcttctgtagattcaaagcataaaacatttgaattcattgctccagggaatgaaattccagagtggtacaaccaccaaagcgtggggccttttataactctagagctgcatcctggttggtttactaacaagtttatgggattcgccacctgtgtcatttttagaatggtgaaaccactcccgcctgcagtcgatgacttgtcgatctgtatgttcttgaatattattggagcaaacgaagtgcatggctttggtttaaactttgagtgtggggaaaagtgcgatcaacctaagttggatcacatatggtttttctatatgcatcgtgatacatactcttattttgagtggcaggacatctactatcagcttgtattctcattcccaccctactactcggtgcgaacagaaagcaacctaacaggtcatgaatgggagtttgcagggatggatgaggtgcaagtgaagaagtgtggtgtgcgtgtggtctatgaggaagatgtggaggagcagagcaattttggctttaataccaagcgaggccttcaacacgaagatgaagaagagggtgaagcatctagtagtgcaagtcaggctcatccaaaaagaaccaaacaactccatcttgacggcagttga

SEQ ID NO: 9 - protein, 1205 aa, Fragaria X ananassa; identical to SEQ ID NO: 7

SEQ ID NO: 10 - DNA, 3606 bp, Fragaria X ananassa
  source 1..3606: mol_type = other DNA
atggaaatgagcattcaaagaacctctacatctttctccccttcaaatcctcagtggaaatatgatgtctttctgagctttagaggtgatgacacccgcaagactttcacagaccacttatacactgcattggaacatcaaggaatcataactttcagggatgatcctgaacttaaaaaagggaaagctatttctccagaacttttttctgcaattaaagaatcaagatttgctctcattattctgtcacaaaattatgcatcgtcaacatggtgcttggatgaacttgtaagagttcttgaatgcatgaaagcaagagaaactgtgctgccaattttctatgatattgatccctctgatgtacgaaagcaaactggtagttttggagaagccttcactcatcacgaagaaaggtttagggatgacaaagagaaagtgcgaaggtggagagatgctttaactgaagtggcaagtttctctgggtggaattcaaaggaatggtatgaatcaaagctcattacaactattgttgaagttatatggacaaaattgcaacctacatcattcagttatgcagaaaatctagttgggattgattcaagattgcagcaagtcaatttgcttttaggtgtaggggtggatgatgtccgcttcattgggatatttggaatgggcgggattggtaagacaactatggtaagagcggtgtatgagagaatcgctcgtgaatttgaatttagtttccttctcagcgatgttagaaactctgttgagaaaagtggtctacttaatttacaaaagcaacttctctcggggatttggagaaagaaggcagagataacggaccttcatggaggagccaccattataaggagattgttaggtcacaaaaaagttcttctcattcttgatgatgtgaaccattcaagccatttaaaatatttggcaggaaaccaagagtggtttggttcagggagtagagttctaatcacaactagaaatgagcatttgttgattgaacatggagtggagagaagattgatggtggaagaattaaatgatgatgattctcttcagctctttagctggaaagcgttcaaaagaggttacactgaagaagattttcttggtttgtccaaatctgttgtaagttatgccaaaggtcttcctttagctcttgaagtactgggttcttttttgcatggaagagatgtaactgaatggaagagtgcactgagaaaactgggaagagtctgtaacttggaaatttttgatgtgctaaaaataagttataaggatctagattctgaagagaagaaaatattcctagacattgcatgtttctttaatggacaggacaaagatcgagttactgaagtattaaacagttgcaatgtctctgcaattattggaataaaagtcctgatggaaagatctttgttgactgtttcatatgggagacttaggatgcacgatttgctccaagaaatgggacgggaaattgtccgctcggaatatcctaacgagccgggcaggtgcagtaggttgtggcttgttgaagacatcaaaaatgtcttaaccaataataccggaactgaagcaatagaaggcatatttgtggactccactgagtcggtagcagaggtggacgtgactccgaaatcattttcaatgatgaacaaattgagatacctgaagattaaaaatgggaacctacctaaggggctggaatatcttcccaatagtttactgattcttgattggactaggtatccgtcaaagtctctaccatcacatttcaaccctcagaagctgctggaacttagcttgtgtcacagctctattaaacatgtccggataggaacagagcctttatacaatttgaaaactgttgatctgagttactctctggatcttgtcaacaccccaaactttaaaagtatgccacatctagaggttttgtttcttgaaggttgtacaagattgtatgaggttgacccaacaattgaagtgcttgaaagacttactgtgcttaacttgaaagattgcaagaatctggtgcattttgcaagcagtgtaagtggcttaaaatctctcaaagttcttaatctttctggttgttcaaaaattaacaaactaccacatgacacgggtcatttggcgagcttggaggagctttatctgaatgacactggcataagggaactgcctacgtctactggaatgcttgaaagactttctctgctaaacatggaagattgcaaacattttgcgtgtcttcccatcagtgtaggtggtttaaaatctctgaaagatctcaatctgtctggttgctcaaagcttgacaaattgccagacgagctgagtcatgtttcctgtttagagaagcttgatgtgagtggaagtggcataagagaagtgccctcctttatcggtcttttgaaaaacctcaaaagattatctcttgctggatgtaaagcagtgtcacctaaatcatggagtataatgttcaacccctttcagttattgcaaaaaagagcttgcgttcccgcaggattgtcactggcttgtttgtctggtttgcattctttaaccaaactggatctaagtgactgtaatctttctgaagaagcaatccccagtgattttggatgctcgttagcctcattgaagatattacatttgagcaacaatcaatttattagactaccagagagcatcggccaactctctagacttgaatctctttacttgaaagggtgccgcaagcttcagacattaccagagtttcgatctgatgtacatgtagacgctctcaactgcacttcattagattcattggtcaatcagataggacaacgcaattcggtgctacgagcgagatgtgttaactgttttaaaatggtggagaataatgatagctataagagcagtgcaatttcattgctaagacgctaccttaagtttcgacgtcagagttcttctgtagattcaaagcataaaacatttgaattcattgctccagggaatgaaattccagagtggtacaaccaccaaagcgtggggccttttataactctagagctgcatcctggttggtttactaacaagtttatgggattcgccacctgtgtcagttttagaatggtgaaaccactcccgcctgcagtcgatgacttttcgatcagtatgctcttgaatattaatggagcatacaaagtgcatggctttgctataaattttgagtttggggaaaagtgcgatcaacctaagttggatcacatatggtttttctatatgcaccgtgatacatactcttattttgagtggcaggacatctactatcagcttgtattcacagtccactgctcggtgccaacagaaagccgcctaacaggtcatgaatgggagtttgcagggatggatgaggtgcaagtgaagaagtgtggtgtgcgtctggtctatgaggaagatgtggaggagcagagcaattttggctttaataccaagcgaggccttcaacacgaagatgaagaagaggatgaagcatctagtgcaagtcaggctcatccaaaaagaaccaaacaactccatcttgacggcagt

SEQ ID NO: 11 - protein, 1202 aa, Fragaria X ananassa; identical to SEQ ID NO: 2

SEQ ID NO: 12 - DNA, 2949 bp, Fragaria X ananassa
  source 1..2949: mol_type = other DNA
atgttgttcctccggcaacggcaactccctccggttctccttctctctctcctcttcctctcctcactctctccctccacatcaaacgagctcgaacaactcttgaagatcaaagcctcacttgaaacttcaaacccaactctgttcagttcatggaaccaagaaactcccacgtgcaacttcactgggattgtctgcaactcaaatgagctcgtctcagaaatcaatctttctcagcagaacctctctggctctcttcctttcgactcattatgttcccttcctgaactcaaaaagctctcttttggttcaaattttctacacggaagcctaacagaggatttgaaaaagtgtactaatttgcaggaacttgatttgggtaagaattctttcactggagaagtgcctgagttgggttctttgagtcagttgaggctgctgagtctgaatgctagtggggtttcagggctttttccatggaagtcacttgggaatctgacgaagttggcttttctgagccttggtgataatgtattcgatcaaagtccgattacaatcgagttgtcaaagcttgacaagctttactggctttacctctcaaactgtagcatcacaggtgaaatcccaaatggtattggaaacctgactctgcttcagaatctggagcttgctgataaccaattggttggtgaaattcctcagagtattataaatctcaacaagctttggcagcttgagctttatggtaattctctgagtgggaaaatcccagttgggtttggcaacctcacaagcctcaggaactttgatgcttcaaaaaacaagcttgagggtgatctctctgagctcaagtccttgacagggcttgtgtctctgcagctgcttgagaatcaattagtcggcgaagtcccggaagagtttggagagttcaagaaccttgttgagctctccctttacaccaacaagctcactggctctcttcctcagaagcttggttcttggtctggcatggagtacattgatgtttcagagaactatttgactggtcctataccccctgggatgtgcaagaatgatagaatgattgatattcttctgctgcagaacaatttcacaggtgggattcctgaatcctatgcagattgcaagtctttggtccggttacgtgtgaataacaattcacttgcgggtactgtccctgctgggatttggagcttgccaaatgttactattcttgatctctctatgaatcaatttgaaggtcccctggcttcagatgttggtaaggctaattctctgtcattattactgttaaataataatcgattttcgggtgaactgcctgctgctttgtctgaagctacatcattggttacaattcagcttagtgagaaccaatttgagggtcaaattccatcaacaattgggaacttgaagaaattaggtagtcttcatttagagcataatatgttgtctagtgccataccggactcactaggctcatgtgttgggatttctgaaatcagccttgcccataacattctctctggccaaatcccaccaagtcttggatcattgcctaacttgaactccttgaacttatctggaaaccagctttctagtgaagttcctgcaactttgtcatctctcaaactcagccttcttgatcttacaaacaaccagttgtctggccgcgtgcccgagtcactatccattgcagcttttaatgaaagctttgctggaaatcctggtttgtgcagccaaaccatgaagaatttgcggccttgttcttcaaattcggactctcctagtcacccaaggatatttttatcttcttttattgctggagtacttcttctgcttattttgcttggtgtgttcttgctatggaagttaaggaggagcgatcttgatcaaccattgaagagtaattcatggactatgaagcagtaccatgtgttaagcttcacagagaatgaaatcctggagtcaatcaaagctgaaaacttgattggcaagggagggtcaggaaatgtgtacaaagtacaactgagagatgggaaagcacttgctgtgaagcacatctggacctcagcagactctgctaaccagaaaacttatcgaagcactacttccattttgaagaagcctaaaacaagctcatcggagtatgatgccgaggtggccacactcagctcattgaggcatgtgaatgtggtgaagctatactgcagcataagtagtgaggacagcaagctgctggtttacgagtactttcctaatgggagcttatgggatcagctgcacatgaataagaaggccaagatggggtgggaagtgaggtatgagattgctttgggggctgcaagaggattggagtatctccaccatggtagtcacagacctgttatacatagagatgtgaagtctagtaatattctgctagatggagattggaagcctagaattgctgattttgggcttgccaagattgtgcaggctggtggtgaaatgactcatgtcattgctgggacacatggctacatagctcctgaatatgcatatacctacaaggtgaacgagaagagtgatgtgtatagctttggggttgtcttgatggagttggtgacaggaaagaggccagtggagcctgagtttggggagaacaaggacatagtgagctgggtttgcagtaaaataatttgcaaagatactgagcttgaattggtggactcgagcatcgaagaggctctaaaagaatatgccttcaacgttttgagcattgcaattcgctgcaccgctagagttccggttctaagaccctcaatgagaatggtggttcaaatgctggaagaggcacaaccttgtcagctcaccagcataagcatcaccaaggaaggtgaaagtaacccgaaaatggtgcagaatttccactacggaaaaatgtag

SEQ ID NO: 13 - protein, 982 aa, Fragaria X ananassa
MLFLRQRQLPPVLLLSLLFLSSLSPSTSNELEQLLKIKASLETSNPTLFSSWNQETPTCNFTGIVCNSNELVSEINLSQQNLSGSLPFDSLCSLPELKKLSFGSNFLHGSLTEDLKKCTNLQELDLGKNSFTGEVPELGSLSQLRLLSLNASGVSGLFPWKSLGNLTKLAFLSLGDNVFDQSPITIELSKLDKLYWLYLSNCSITGEIPNGIGNLTLLQNLELADNQLVGEIPQSIINLNKLWQLELYGNSLSGKIPVGFGNLTSLRNFDASKNKLEGDLSELKSLTGLVSLQLLENQLVGEVPEEFGEFKNLVELSLYTNKLTGSLPQKLGSWSGMEYIDVSENYLTGPIPPGMCKNDRMIDILLLQNNFTGGIPESYADCKSLVRLRVNNNSLAGTVPAGIWSLPNVTILDLSMNQFEGPLASDVGKANSLSLLLLNNNRFSGELPAALSEATSLVTIQLSENQFEGQIPSTIGNLKKLGSLHLEHNMLSSAIPDSLGSCVGISEISLAHNILSGQIPPSLGSLPNLNSLNLSGNQLSSEVPATLSSLKLSLLDLTNNQLSGRVPESLSIAAFNESFAGNPGLCSQTMKNLRPCSSNSDSPSHPRIFLSSFIAGVLLLLILLGVFLLWKLRRSDLDQPLKSNSWTMKQYHVLSFTENEILESIKAENLIGKGGSGNVYKVQLRDGKALAVKHIWTSADSANQKTYRSTTSILKKPKTSSSEYDAEVATLSSLRHVNVVKLYCSISSEDSKLLVYEYFPNGSLWDQLHMNKKAKMGWEVRYEIALGAARGLEYLHHGSHRPVIHRDVKSSNILLDGDWKPRIADFGLAKIVQAGGEMTHVIAGTHGYIAPEYAYTYKVNEKSDVYSFGVVLMELVTGKRPVEPEFGENKDIVSWVCSKIICKDTELELVDSSIEEALKEYAFNVLSIAIRCTARVPVLRPSMRMVVQMLEEAQPCQLTSISITKEGESNPKMVQNFHYGKM

SEQ ID NO: 14 - DNA, 2949 bp, Fragaria X ananassa
  source 1..2949: mol_type = other DNA
atgtcgttcctccggcaacggcaacttcctccggttctccttctctctctcctcttcctctcctcactctctccctccacatcaaacgagctcgaccaactcttgaagatcaaagcctcacttgaaacttcaaacccaacactgttcagttcatggaaccaagaaactcccacgtgcaacttcactgggattgtctgcaactcaaatgagctcgtctcagaaatcaatctttctcagcagaacctctctggctctcttcctttcgacgcattatgttcccttcctgaactcaaaaagctctcttttggttcaaattttctacacggaagcctaacagaggatttgaaaaagtgtactaatttgcaggaacttgatttgggtaagaattctttcactggagaagtgcctgagttgggttctttgagtcagttgaggctgctgagtctgaatgctagtggggtttcagggctttttccatggaagtcacttgggaatctgacgaagttggcttttttgagccttggtgataatgtattcgatcaaagtccgattacaatggagttgtcaaagcttgacaagctttactggctttacctctcaaactgtagcatcacaggtgaaatcccaaatggtattggaaacctgactctgcttcagaatctggagcttgctgataaccaattggttggtgaaattcctcagagtattataaatctcaacaagctttggcagcttgagctttatggtaattctctgagtgggaaaatcccagttgggtttggcaacctcacaagcctcaggaactttgatgcttcaataaacaagcttgagggtgatctctctgagctcaagtccttgacagggcttgtgtctctgcagctgcttgagaatcaattagtcggcgaagtcccggaagagtttggagagttcaagaaccttgttgagctctccctttacaccaacaagctcactggctctcttcctcagaagcttggttcttggtctggcatggagtacattgatgtttcagagaactatttgactggtccaataccccctgggatgtgcaagaatgatagaatgattgatattcttctgctgcagaacaatttcacaggtgggattcctgaatcctatgcagattgcaagtctttggtccggttacgtgtgaataacaattcactttcgggtactgtccctgctgggatttggagcttgccaaatgttactattcttgatctctctatgaatcaatttgaaggtcccctggcttcagatgttggtaaggctaattctctgtcattattactgttaaataataatcgattttcgggtgaactgcctgctgctttgtctgaagctacatcattggttacaattcagcttagtgagaaccaatttgagggtcaaattccatcaacaattgggaacttgaagaaattaggtagtcttcatttagagcataatatgttgtctggtgccataccggactcactaggctcatgtgttaggatttctgaaatcagccttgcccataacattctctctggccaaatcccaccaagtcttggatcattgcctaacttgaactccttgaacttatctggaaaccagctttctagtgaagttcctgcaactttgtcatctctcaaactcagccttcttgatcttacaaacaaccagttgtctggccgcgtgcccgagtcactatccattgcagcttttaatgaaagctttgctggaaatcctggtttgtgcagccaaaccatgaagaatttgcggccttgttcttcaaattcggactctcctagtcacccaaggatatttttatcttcttttattgctggagtacttcttctgcttattttgcttggtgtgttcttgctatggaagctaaggaggagcgatcttgatcaaccattgaagagtaattcatggactatgaagcagtaccatgtgttaagcttcacagagaatgaaatcctagagtcaatcaaagctgaaaacttgattggcaagggagggtcaggaaatgtgtacaaagtacaactgagagatgggaaagcacttgctgtgaagcacatctggacctcagcagactatgctaaccagaaaacttatcgaagcactacttccattttgaagaagcctaaaacaagctcatcggagtatgatgccgaggtggccacactcagctcattgaggcatgtgaatgtggtgaagctatactgcagcataagtagtgaggacagcaagctgctggtttacgagtactttcctaatgggagcttatgggatcagctgcacatgaataagaaggccaagatggggtgggaagtgaggtatgagattgctttgggggctgcaagaggattggagtatctccaccatggtagtcacagacctgttatacatagagatgtgaagtctagtaatattctgctagatggagattggaagcctagaattgctgattttgggcttgccaagattgtgcaggctggtggtgaaatgactcatgtcattgctgggacacatggctacatagctcctgaatatgcatatacctacaaggtgaacgagaagagtgatgtgtatagctttggggttgtcttgatggagttggtgacaggaaagaggccagtggagcctgagtttggggagaacaaggacatagtgagctgggtttgcagtaaaataatttgcaaagatactgagcttgaattggtggactcgagcatcgaagaggctctaaaagaatatgccctcaacgttttgagcattgcaattcgctgcaccgctagagttccggttctaagaccctcaatgagaatggtggttcaaatgctggaagaggcacaaccttgtcagctcaccagcataagcattaccaaggaaggtgaaagtaacccgaaaatggagcagaatttccactacggaaaaatgtag

SEQ ID NO: 15 - protein, 982 aa, Fragaria X ananassa
MSFLRQRQLPPVLLLSLLFLSSLSPSTSNELDQLLKIKASLETSNPTLFSSWNQETPTCNFTGIVCNSNELVSEINLSQQNLSGSLPFDALCSLPELKKLSFGSNFLHGSLTEDLKKCTNLQELDLGKNSFTGEVPELGSLSQLRLLSLNASGVSGLFPWKSLGNLTKLAFLSLGDNVFDQSPITMELSKLDKLYWLYLSNCSITGEIPNGIGNLTLLQNLELADNQLVGEIPQSIINLNKLWQLELYGNSLSGKIPVGFGNLTSLRNFDASINKLEGDLSELKSLTGLVSLQLLENQLVGEVPEEFGEFKNLVELSLYTNKLTGSLPQKLGSWSGMEYIDVSENYLTGPIPPGMCKNDRMIDILLLQNNFTGGIPESYADCKSLVRLRVNNNSLSGTVPAGIWSLPNVTILDLSMNQFEGPLASDVGKANSLSLLLLNNNRFSGELPAALSEATSLVTIQLSENQFEGQIPSTIGNLKKLGSLHLEHNMLSGAIPDSLGSCVRISEISLAHNILSGQIPPSLGSLPNLNSLNLSGNQLSSEVPATLSSLKLSLLDLTNNQLSGRVPESLSIAAFNESFAGNPGLCSQTMKNLRPCSSNSDSPSHPRIFLSSFIAGVLLLLILLGVFLLWKLRRSDLDQPLKSNSWTMKQYHVLSFTENEILESIKAENLIGKGGSGNVYKVQLRDGKALAVKHIWTSADYANQKTYRSTTSILKKPKTSSSEYDAEVATLSSLRHVNVVKLYCSISSEDSKLLVYEYFPNGSLWDQLHMNKKAKMGWEVRYEIALGAARGLEYLHHGSHRPVIHRDVKSSNILLDGDWKPRIADFGLAKIVQAGGEMTHVIAGTHGYIAPEYAYTYKVNEKSDVYSFGVVLMELVTGKRPVEPEFGENKDIVSWVCSKIICKDTELELVDSSIEEALKEYALNVLSIAIRCTARVPVLRPSMRMVVQMLEEAQPCQLTSISITKEGESNPKMEQNFHYGKM

SEQ ID NO: 16 - DNA, 2946 bp, Fragaria X ananassa
  source 1..2946: mol_type = other DNA
atgttgttcctccggcaacggcaactccctccagttctccttctctcgctcctcttcctctcctcactctctccctccacatcaaacgagctcgaccaactcttgaagatcaaagcctcacttgaaacttcaaacccaactctgttcagttcatggaaccaagaaactcccacgtgcaacttcactgggattgtctgcaactcaaatgagctcgtctcagaaatcaatctttctcagcagaacctctctggctctcttcctttcgacgcattatgttcccttcctgaactcaaaaagctctcttttggttcaaattttctacacggaagcctgacagaggatttgaaaaagtgtactaatttgcaggaacttgatttgggtaagaattctttcactggagaagtgcctgagttgggttctttgagtcagttgaggctgctgagtctgaatgctagtggggtttcagggctttttccatggaagtcacttgggaatctgacgaagttggcttttttgagccttggtgataatgtattcgatcaaagtccgattacaatggagttgtcaaagcttgacaagctttactggctttacctctcaaactgtagcatcacaggtgaaatcccaaatggtattggaaacctgactctgcttcagaatctggagcttgctgataaccaattggttggtgaaattcctcagagtattataaatctcaacaagctttggcagcttgagctttatggtaattctctgagtgggaaattcccagttgggtttggcaacctcacaagcctcaggaactttgatgcttcaaaaaacaagcttgagggtgatctctctgagctcaagtccttgacagggcttgtgtctctgcagctgcttgagaatcaattagtcggcgaagtcccggaagagtttggggagttcaagaaccttgttgagctctccctttacaccaacaagcttactggctctcttcctcagaagcttggttcttggtctggcatggagtatattgatgtttcagagaactatttgactggtcctataccccctgggatgtgcaagaatgatagaatgattgatattcttctgctgcagaacaatttcacaggtgggattcctgaatcctatgcagattgcaagtctttggtccggttacgtgtgaataacaattcactttcgggtactgtccctgctgggatttggagcttgccaaatgttactattcttgatctctctatgaatcaatttgaaggtcccctggcttcagatgttggtaaggctaattctctgtcattattactgttaaataataatcgattttggggtgaactgcctgctgctttgtctgaagctacatcattggttacaattcagcttagtgagaaccaatttgagggtcaaattccatcaacaattgggaacttgaagaaattaggtagtcttcatttagagcataatatgttgtctggtgccataccagactcactaggttcatgtgttaggatttctgaaatcggccttgcccataacattctctctggccaaatcccaccaagtcttggatcattgcctaacttgaactctttgaacttatctggaaaccagctttctagtgaagttcctacaactttgtcatctctcaaactcagccttcttgatcttacaaacaaccagttgtctggccgcgtgcccgagtcactatccattgcagcttttaatgaaagctttgctggaaatcctggtttgtgcagccaaaccatgaagaatttgcggccttgttcttcaaattcagactctcctagtcacccaaggatatttttgtcttcttttattgctggagtacttcttctgcttattttgcttggtgtgttcttgctatggaagctaaggaggagcgatcttgatcaaccattgaagagtaattcatggactatgaagcagtaccatgtgttaagcttcacagagaatgaaatcctagagtcaatcaaagctgaaaacttgattggcaagggagggtcaggaaatgtgtacaaagtacaactgagagatgggaaagcacttgctgtgaagcacatctggacctcagcagactctgctaaccagaaaacttatcgaagcactacttccattttgaagaagcctaaaacaagctcatcggagtatgatgccgaggtggccacactcagctcattgaggcatgtgaatgtggtgaagctatactgcagcataagtagtgaggacagcaagctgctggtttacgagtactttcctaatgggagcttatgggatcagctgcacatgaataagaaggccaagatggggtgggaagtgaggtatgagattgctttgggggctgcaagaggattggagtatctccaccatggtagtcacagacctgttatacatagagatgtgaagtctagtaatattctgctagatggagattggaagcctagaattgctgattttgggcttgccaagattgtgcaggctggtggtgaaatgactcatgtcattgctgggacacatggctacatagctcctgaatatgcatatacctacaaggtgaacgagaagagtgatgtgtatagctttggggttgtcttgatggagttggtgacaggaaagaggccagtggagcctgagtttggggagaacaaggacatagtgagctgggtttgcagtaaaataatttgcaaagatactgagcttgaattggtggactcgagcatcgaagaggctctaaaagaatatgccctcaacgttttgagcattgcaattcgctgcaccgctagagttccggttctaagaccctcaatgagaatggtggttcaaatgctggaagaggcacaaccttgtcagctcaccagcataagcatcaccaaggaaggtgaaagtaacccgaaaatggagcagaatttccactacggaaaaatg

SEQ ID NO: 17 - protein, 982 aa, Fragaria X ananassa; identical to SEQ ID NO: 4

SEQ ID NO: 18 - DNA, 21 bp, synthetic construct
  source 1..21: mol_type = other DNA
gctgttgtcttctcgattact

SEQ ID NO: 19 - DNA, 19 bp, synthetic construct
  source 1..19: mol_type = other DNA
caagttcatccaagcacca

SEQ ID NO: 20 - DNA, 23 bp, synthetic construct
  source 1..23: mol_type = other DNA
ggcaagtaggtgtcaatggaaat

SEQ ID NO: 21 - DNA, 20 bp, synthetic construct
  source 1..20: mol_type = other DNA
ctccaccttcgcactttctc

SEQ ID NO: 22 - DNA, 20 bp, synthetic construct
  source 1..20: mol_type = other DNA
gtattcacagtccactgctc

SEQ ID NO: 23 - DNA, 20 bp, synthetic construct
  source 1..20: mol_type = other DNA
actcccattcatgacctgtt

SEQ ID NO: 24 - DNA, 19 bp, synthetic construct
  source 1..19: mol_type = other DNA
tgccaacagaaagccgcct

SEQ ID NO: 25 - DNA, 18 bp, synthetic construct
  source 1..18: mol_type = other DNA
atgcgctgattgaatctc

SEQ ID NO: 26 - DNA, 19 bp, synthetic construct
  source 1..19: mol_type = other DNA
tggcttccggttcaaatag

SEQ ID NO: 27 - DNA, 18 bp, synthetic construct
  source 1..18: mol_type = other DNA
agctggagtagcgagtcg

SEQ ID NO: 28 - DNA, 4025 bp, Fragaria X ananassa
  source 1..4025: mol_type = genomic DNA
atggaaatgagcattcaaagaacctctacatctttctccccttcaaatcctcagtggaaatatgatgtctttctgagctttagaggtgatgacacccgcaagactttcacagaccacttatacactgcattggaacatcaaggaatcataactttcagggatgatcctgaacttaaaaaagggaaagctatttctccagaacttttttctgcaattaaagaatcaagatttgctctcattattctgtcacaaaattatgcatcgtcaacatggtgcttggatgaacttgtaagagttcttgaatgcatgaaagcaagagaaactgtgctgccaattttctatgatattgatccctctgatgtacgaaagcaaactggtagttttggagaagccttcactcatcacgaagaaaggtttagggatgacaaagagaaagtgcgaaggtggagagatgctttaactgaagtggcaagtttctctgggtggaattcaaaggaatggtatgcatatactcttttatttttgtctcatcaatctacagtggttgattttggtgctcttcattatttttttcacaaataatctagtcagatagtcaatctgatagtttatgaggcttttttgtcattactgaaatataaaacaaattgcagatacattttcattaactagatcttcatatcaataacttgatacttttattcttttatatgtaataggtatgaatcaaagctcattacaactattgttgaagttatatggacaaaattgcaacctacatcattcagttatgcagaaaatctagttgggattgattcaagattgcagcaagtcaatttgcttttaggtgtaggggtggatgatgtccgcttcattgggatatttggaatgggcgggattggtaagacaactatggtaagagcggtgtatgagagaatcgctcgtgaatttgaatttagtttccttctcagcgatgttagaaactctgttgagaaaagtggtctacttaatttacaaaagcaacttctctcggggatttggagaaagaaggcagagataacggaccttcatggaggagccaccattataaggagattgttaggtcacaaaaaagttcttctcattcttgatgatgtgaaccattcaagccatttaaaatatttggcaggaaaccaagagtggtttggttcagggagtagagttctaatcacaactagaaatgagcatttgttgattgaacatggagtggagagaagattgatggtggaagaattaaatgatgatgattctcttcagctctttagctggaaagcgttcaaaagaggttacactgaagaagattttcttggtttgtccaaatctgttgtaagttatgccaaaggtcttcctttagctcttgaagtactgggttcttttttgcatggaagagatgtaactgaatggaagagtgcactgagaaaactgggaagagtctgtaacttggaaatttttgatgtgctaaaaataagttataaggatctagattctgaagagaagaaaatattcctagacattgcatgtttctttaatggacaggacaaagatcgagttactgaagtattaaacagttgcaatgtctctgcaattattggaataaaagtcctgatggaaagatctttgttgactgtttcatatgggagacttaggatgcacgatttgctccaagaaatgggacgggaaattgtccgctcggaatatcctaacgagccgggcaggtgcagtaggttgtggcttgttgaagacatcaaaaatgtcttaaccaataataccgtaagagatttagaagaagaatcatttgaattgttattttctaatatattatatgaccagtcagtctactaaatctgctttttattgtatgatgacaatgggttaatagggaactgaagcaatagaaggcatatttgtggactccactgagtcggtagcagaggtggacgtgactccgaaatcattttcaatgatgaacaaattgagatacctgaagattaaaaatgggaacctacctaaggggctggaatatcttcccaatagtttactgattcttgattggactaggtatccgtcaaagtctctaccatcacatttcaaccctcagaagctgctggaacttagcttgtgtcacagctctattaaacatgtccggataggaacagaggtatactattattactattttaacgtataatgattgttgattatatcatggaagctaattttttctatgctttttacttgacgtgcagcctttatacaatttgaaaactgttgatctgagttactctctggatcttgtcaacaccccaaactttaaaagtatgccacatctagaggttttgtttcttgaaggttgtacaagattgtatgaggttgacccaacaattgaagtgcttgaaagacttactgtgcttaacttgaaagattgcaagaatctggtgcattttgcaagcagtgtaagtggcttaaaatctctcaaagttcttaatctttctggttgttcaaaaattaacaaactaccacatgacacgggtcatttggcgagcttggaggagctttatctgaatgacactggcataagggaactgcctacgtctactggaatgcttgaaagactttctctgctaaacatggaagattgcaaacattttgcgtgtcttcccatcagtgtaggtggtttaaaatctctgaaagatctcaatctgtctggttgctcaaagcttgacaaattgccagacgagctgagtcatgtttcctgtttagagaagcttgatgtgagtggaagtggcataagagaagtgccctcctttatcggtcttttgaaaaacctcaaaagattatctcttgctggatgtaaagcagtgtcacctaaatcatggagtataatgttcaacccctttcagttattgcaaaaaagagcttgcgttcccgcaggattgtcactggcttgtttgtctggtttgcattctttaaccaaactggatctaagtgactgtaatctttctgaagaagcaatccccagtgattttggatgctcgttagcctcattgaagatattacatttgagcaacaatcaatttattagactaccagagagcatcggccaactctctagacttgaatctctttacttgaaagggtgccgcaagcttcagacattaccagagtttcgatctgatgtacatgtagacgctctcaactgcacttcattagattcattggtcaatcagataggacaacgcaattcggtgctacgagcgagatgtgttaactgttttaaaatggtggagaataatgatagctataagagcagtgcaatttcattgctaagacgctaccttaagtttcgacgtcagagttcttctgtagattcaaagcataaaacatttgaattcattgctccagggaatgaaattccagagtggtacaaccaccaaagcgtggggccttttataactctagagctgcatcctggttggtttactaacaagtttatgggattcgccacctgtgtcagttttagaatggtgaaaccactcccgcctgcagtcgatgacttttcgatcagtatgctcttgaatattaatggagcatacaaagtgcatggctttgctataaattttgagtttggggaaaagtgcgatcaacctaagttggatcacatatggtttttctatatgcaccgtgatacatactcttattttgagtggcaggacatctactatcagcttgtattcacagtccactgctcggtgccaacagaaagccgcctaacaggtcatgaatgggagtttgcagggatggatgaggtgcaagtgaagaagtgtggtgtgcgtctggtctatgaggaagatgtggaggagcagagcaattttggctttaataccaagcgaggccttcaacacgaagatgaagaagaggatgaagcatctagtgcaagtcaggctcatccaaaaagaaccaaacaactccatcttgacggcagttga

SEQ ID NO: 29 - DNA, 3090 bp, Fragaria X ananassa
  source 1..3090: mol_type = genomic DNA
atgttgttcctccggcaacggcaactccctccagttctccttctctcgctcctcttcctctcctcactctctccctccacatcaaacgagctcgaccaactcttgaagatcaaagcctcacttgaaacttcaaacccaactctgttcagttcatggaaccaagaaactcccacgtgcaacttcactgggattgtctgcaactcaaatgagctcgtctcagaaatcaatctttctcagcagaacctctctggctctcttcctttcgacgcattatgttcccttcctgaactcaaaaagctctcttttggttcaaattttctacacggaagcctgacagaggatttgaaaaagtgtactaatttgcaggaacttgatttgggtaagaattctttcactggagaagtgcctgagttgggttctttgagtcagttgaggctgctgagtctgaatgctagtggggtttcagggctttttccatggaagtcacttgggaatctgacgaagttggcttttttgagccttggtgataatgtattcgatcaaagtccgattacaatggagttgtcaaagcttgacaagctttactggctttacctctcaaactgtagcatcacaggtgaaatcccaaatggtattggaaacctgactctgcttcagaatctggagcttgctgataaccaattggttggtgaaattcctcagagtattataaatctcaacaagctttggcagcttgagctttatggtaattctctgagtgggaaattcccagttgggtttggcaacctcacaagcctcaggaactttgatgcttcaaaaaacaagcttgagggtgatctctctgagctcaagtccttgacagggcttgtgtctctgcagctgcttgagaatcaattagtcggcgaagtcccggaagagtttggggagttcaagaaccttgttgagctctccctttacaccaacaagcttactggctctcttcctcagaagcttggttcttggtctggcatggagtatattgatgtttcagagaactatttgactggtcctataccccctgggatgtgcaagaatgatagaatgattgatattcttctgctgcagaacaatttcacaggtgggattcctgaatcctatgcagattgcaagtctttggtccggttacgtgtgaataacaattcactttcgggtactgtccctgctgggatttggagcttgccaaatgttactattcttgatctctctatgaatcaatttgaaggtcccctggcttcagatgttggtaaggctaattctctgtcattattactgttaaataataatcgattttggggtgaactgcctgctgctttgtctgaagctacatcattggttacaattcagcttagtgagaaccaatttgagggtcaaattccatcaacaattgggaacttgaagaaattaggtagtcttcatttagagcataatatgttgtctggtgccataccagactcactaggttcatgtgttaggatttctgaaatcggccttgcccataacattctctctggccaaatcccaccaagtcttggatcattgcctaacttgaactctttgaacttatctggaaaccagctttctagtgaagttcctacaactttgtcatctctcaaactcagccttcttgatcttacaaacaaccagttgtctggccgcgtgcccgagtcactatccattgcagcttttaatgaaagctttgctggaaatcctggtttgtgcagccaaaccatgaagaatttgcggccttgttcttcaaattcagactctcctagtcacccaaggatatttttgtcttcttttattgctggagtacttcttctgcttattttgcttggtgtgttcttgctatggaagctaaggaggagcgatcttgatcaaccattgaagagtaattcatggactatgaagcagtaccatgtgttaagcttcacagagaatgaaatcctagagtcaatcaaagctgaaaacttgattggcaagggagggtcaggaaatgtgtacaaagtacaactgagagatgggaaagcacttgctgtgaagcacatctggacctcagcagactctgctaaccagaaaacttatcgaagcactacttccattttgaagaagcctaaaacaagctcatcggagtatgatgccgaggtggccacactcagctcattgaggcatgtgaatgtggtgaagctatactgcagcataagtagtgaggacagcaagctgctggtttacgagtactttcctaatgggagcttatgggatcagctgcacatgaataagaaggccaagatggggtgggaagtgaggtatgagattgctttgggggctgcaagaggattggagtatctccaccatggtagtcacagacctgttatacatagagatgtgaagtctagtaatattctgctagatggagattggaagcctagaattgctgattttgggcttgccaagattgtgcaggctggtggtgaaatgactcatgtcattgctgggacacatggctacatagctcctggtaaattctaattatattactatctttgttcttatttagatttgtagtttagattgatagtctcgcttagtcgcaacattacatgtcaaatgaacttatgagactgaaatgattgaacttcttgatgttttggtggttcagaatatgcatatacctacaaggtgaacgagaagagtgatgtgtatagctttggggttgtcttgatggagttggtgacaggaaagaggccagtggagcctgagtttggggagaacaaggacatagtgagctgggtttgcagtaaaataatttgcaaagatactgagcttgaattggtggactcgagcatcgaagaggctctaaaagaatatgccctcaacgttttgagcattgcaattcgctgcaccgctagagttccggttctaagaccctcaatgagaatggtggttcaaatgctggaagaggcacaaccttgtcagctcaccagcataagcatcaccaaggaaggtgaaagtaacccgaaaatggagcagaatttccactacggaaaaatgtag

SEQ ID NO: 30 - DNA, 21 bp, synthetic construct
  source 1..21: mol_type = other DNA
ttacatatccgtggaggagat

SEQ ID NO: 31 - DNA, 19 bp, synthetic construct
  source 1..19: mol_type = other DNA
gagcgagagaaggagaact

SEQ ID NO: 32 - DNA, 23 bp, synthetic construct
  source 1..23: mol_type = other DNA
gggaacttgaagaaattaggtag

SEQ ID NO: 33 - DNA, 18 bp, synthetic construct
  source 1..18: mol_type = other DNA
aatgttatgggcaaggcc

SEQ ID NO: 34 - DNA, 24 bp, synthetic construct
  source 1..24: mol_type = other DNA
ggtgccataccagactcactaggt

SEQ ID NO: 35 - DNA, 18 bp, synthetic construct
  source 1..18: mol_type = other DNA
aaccctaaccctaattcc

SEQ ID NO: 36 - DNA, 18 bp, synthetic construct
  source 1..18: mol_type = other DNA
gagaggttttggagaagg

SEQ ID NO: 37 - DNA, 20 bp, synthetic construct
  source 1..20: mol_type = other DNA
aaggatctatggcggccacc

SEQ ID NO: 38 - DNA, 19 bp, synthetic construct
  source 1..19: mol_type = other DNA
tgccacgtgtttaaggact

SEQ ID NO: 39 - DNA, 22 bp, synthetic construct
  source 1..22: mol_type = other DNA
cccgaaaacaaacaaatgagac

SEQ ID NO: 40 - DNA, 20 bp, synthetic construct
  source 1..20: mol_type = other DNA
aacaccacgagggccccact

(19) United States
(12) Patent Application Publication
(10) Pub. No.: US 2025/0043304 A1
(43) Pub. Date: Feb. 6, 2025

(54) FUSARIUM WILT RESISTANCE GENES

(71) Applicants: J.R. Simplot Company, Boise, ID (US); Plant Sciences, Inc., Watsonville, CA (US)

(72) Inventors: Jeffrey W. HABIG, Boise, ID (US); Genevieve HALVORSEN, Boise, ID (US); Eric JACKSON, Pocatello, ID (US)

(21) Appl. No.: 18/792,335

(22) Filed: Aug. 1, 2024

Related U.S. Application Data
(60) Provisional application No. 63/517,418, filed on Aug. 3, 2023.

(51) Int. Cl. C12N 15/82; C07K 14/415

(52) U.S. Cl. CPC C12N 15/8282; C07K 14/415

(57) ABSTRACT

The present disclosure relates to cisgenic, transgenic, and genetically engineered plants having resistance to Fusarium wilt. The disclosure further relates to the use of disclosed sequences for marker-assisted breeding to introduce Fusarium wilt resistance via breeding methods. The disclosure also teaches methods for the precise introduction of resistance in any strawberry variety via genetic modification or gene editing approaches. The disclosure further relates to methods of identifying plants having one or more of the resistance alleles.

### CROSS-REFERENCE TO RELATED APPLICATION

This application claims the benefit of priority to U.S. Provisional Application No. 63/517,418, filed Aug. 3, 2023, the entire contents of which is incorporated herein by reference.

### FIELD

The present disclosure relates to methods and compositions for marker-assisted breeding for plants having resistance to fungal pathogens, such as fusarium wilt. Also provided are methods for transforming plants with the disclosed sequences, engineering plants to comprise resistant alleles, detecting said sequences, and plants produced therefrom.

### REFERENCE TO AN ELECTRONIC SEQUENCE LISTING

The contents of the electronic sequence listing (JRSI_088_O1US_SeqList_ST26.xml; Size: 80,686 bytes; and Date of Creation: Jul. 26, 2024) are herein incorporated by reference in its entirety.

### BACKGROUND

Plant diseases such as fusarium wilt are detrimental, causing economic loss of plant crops. Fusarium wilt is a vascular disease caused by the fungal pathogen Fusarium oxysporum, leading to plant stunting, wilting, and vegetative necrosis. F. oxysporum has a wide host range, with specific strains that are genetically distinct and typically host specific. In strawberry, F. oxysporum f. sp. Fragariae is the causal agent of fusarium wilt, which leads to yield loss and has become a significant threat to production. Two races of F. oxysporum f. sp. Fragariae have been identified, with race 1 widely distributed in California, the largest production region in the U.S.

Several management strategies exist to reduce fusarium wilt effects, such as crop rotation and soil fumigation. However, these are costly, harmful to the environment, and/or don't consistently prevent disease. Further, fungicides and other pesticides have strong physiological effects on the plants, may produce residues that are toxic to food crops, can have high animal toxicity, and are potentially hazardous to workers using them.

Therefore, there is a need to identify resistant genes and develop compositions and methods for controlling fungal pathogens.

### SUMMARY OF THE DISCLOSURE

The following embodiments and aspects thereof are described in conjunction with methods, plants, genetic markers, and compositions which are meant to be exemplary, not limiting in scope. In various embodiments, one or more of the above-described problems have been reduced or eliminated, while other embodiments are directed to other improvements.

In some aspects, the techniques described herein relate to a method for conferring Fusarium resistance in a plant, including at least one of: (a) introducing a nucleic acid into a plant, plant part, or plant cell, wherein said nucleic acid encodes an amino acid sequence at least 90% identical to SEQ ID NO: 2, and (b) introducing a nucleic acid into a plant, plant part, or plant cell, wherein said nucleic acid encodes an amino acid sequence at least 90% identical to SEQ ID NO: 4. In some aspects, the techniques described herein relate to a Fragaria spp. plant produced by the method. In some aspects, the expression of the nucleotide sequence in the plant prevents Fusarium from colonizing the plant, or prevents Fusarium from affecting plant growth or marketable yield.

In some aspects, the techniques described herein relate to a cisgenic Fragaria spp. plant produced by the methods disclosed herein, wherein the cisgenic Fragaria spp. plant includes a nucleic acid encoding an amino acid sequence at least 90% identical to SEQ ID NO: 2. In some aspects, the techniques described herein relate to a cisgenic Fragaria spp. plant produced by the methods disclosed herein, wherein the cisgenic Fragaria spp. plant includes a nucleic acid encoding an amino acid sequence at least 90% identical to SEQ ID NO: 4.

In some aspects, the techniques described herein relate to a transgenic Fragaria spp. plant produced by the methods disclosed herein, wherein the transgenic Fragaria spp. plant includes a nucleic acid encoding an amino acid sequence at least 90% identical to SEQ ID NO: 2. In some aspects, the techniques described herein relate to a transgenic Fragaria spp. plant produced by the methods disclosed herein, wherein the transgenic Fragaria spp. plant includes a nucleic acid encoding an amino acid sequence at least 90% identical to SEQ ID NO: 4. In some aspects, the the nucleic acid encoding an amino acid sequence at least 90% identical to SEQ ID NO: 2, and/or the nucleic acid encoding an amino acid sequence at least 90% identical to SEQ ID NO: 4 is operably linked to a constitutive promoter. In some aspects, the transgenic Fragaria spp. plant has between a 1.1 and 20 fold increase in expression of the nucleic acid encoding an amino acid sequence at least 90% identical to SEQ ID NO: 2 and/or the nucleic acid encoding an amino acid sequence at least 90% identical to SEQ ID NO: 4 compared to a non-transgenic Fragaria spp. plant.

In some aspects, the Fragaria spp. plants produced from the methods disclosed herein have a Fusarium wilt resistance rAUDPC score of less than 0.04, less than 0.03, less than 0.02, less than 0.01, or between 0.01 and 0.02.

In some aspects, the techniques described herein relate to a method of producing a Fragaria spp. plant including a Fusarium wilt resistance allele, including: screening a Fragaria spp. plant for the presence of a Fusarium wilt resistance allele designated FUS1, wherein said FUS1 allele encodes a protein including SEQ ID NO: 2 or a sequence at least 90% identical thereto, and wherein the protein is a TIR-NB-ARC-LRR protein that includes a C-terminal Jelly-roll/Ig-like Domain, and selecting a Fragaria spp. plant including said FUS1 allele; crossing the selected Fragaria spp. plant including said FUS1 allele with a second Fragaria spp. plant to produce progeny Fragaria spp. plants; screening said progeny Fragaria spp. plants for the presence of said FUS1 allele; and selecting a progeny Fragaria spp. plant including at least one copy of said FUS1 allele.

In some aspects, the techniques described herein relate to a method, wherein the FUS1 allele encoding a protein having at least 90% identity SEQ ID NO: 2 includes a C-JID sequence having a serine at a position corresponding to 1,046 of SEQ ID NO: 2; a phenylalanine at a position corresponding to 1,060 of SEQ ID NO: 2; a serine at a position corresponding to 1,063 of SEQ ID NO: 2; a leucine at a position corresponding to 1,065 of SEQ ID NO: 2; an asparagine at a position corresponding to 1,069 of SEQ ID NO: 2; a tyrosine at a position corresponding to 1,072 of SEQ ID NO: 2; a lysine at a position corresponding to 1,073 of SEQ ID NO: 2; a phenylalanine at a position corresponding to 1,083 of SEQ ID NO: 2; a valine at a position corresponding to 1,121 of SEQ ID NO: 2; a histidine at a position corresponding to 1,122 of SEQ ID NO: 2; a cysteine at a position corresponding to 1,123 of SEQ ID NO: 2; a proline at a position corresponding to 1,126 of SEQ ID NO: 2; and an arginine at a position corresponding to 1,130 of SEQ ID NO: 2. In some embodiments, the FUS1 allele encoding a protein having at least 90% identity SEQ ID NO: 2 further includes an alanine at a position corresponding to 1,078 of SEQ ID NO:2; an isoleucine at a position corresponding to 1,079 of SEQ ID NO:2; a threonine at a position corresponding to 1,120 of SEQ ID NO: 2; and a leucine at a position corresponding to 1,153 of SEQ ID NO: 2.

In some aspects, the techniques described herein relate to a method using marker-assisted selection, wherein the marker includes a DNA marker selected from the group consisting of SEQ ID NO: 1, cDNA sequences thereof, fragments of at least 18 consecutive nucleotides thereof, and complementary sequences thereof. In some aspects, the marker is selected from the group consisting of SEQ ID NO: 28, cDNA sequences thereof, fragments of at least 18 consecutive nucleotides thereof, and complementary sequences thereof.

In some aspects, the techniques described herein relate to a method of producing a Fragaria spp. plant including a Fusarium wilt resistance allele, including: screening a Fragaria spp. plant for the presence of a Fusarium wilt resistance allele designated FUS4, wherein said FUS4 allele encodes a protein including SEQ ID NO: 4, or a sequence at least 90% identical thereto, and wherein the protein encoded by said allele is a leucine rich receptor like kinase, and selecting a Fragaria spp. plant including said FUS4 allele; crossing the selected Fragaria spp. plant including said FUS4 allele with a second Fragaria spp. plant to produce progeny Fragaria spp. plants; screening said progeny Fragaria spp. plants for the presence of said FUS4 allele; and selecting a progeny Fragaria spp. plant including at least one copy of said FUS4 allele.

In some aspects, the techniques described herein relate to a method, wherein the FUS4 allele encoding a protein having at least 90% identity SEQ ID NO: 4 includes a phenylalanine at a position corresponding to 256 of SEQ ID NO: 4; a tryptophan at a position corresponding to 444 of SEQ ID NO: 4; a glycine at a position corresponding to 509 of SEQ ID NO: 4; and a threonine at a position corresponding to 545 of SEQ ID NO: 4.

In some aspects, the techniques described herein relate to a method using marker-assisted selection, wherein the marker includes a DNA marker selected from the group consisting of SEQ ID NO: 3, cDNA sequences thereof, fragments of at least 18 consecutive nucleotides thereof, and complementary sequences thereof. In some aspects, the marker is selected from the group consisting of SEQ ID NO: 29, cDNA sequences thereof, fragments of at least 18 consecutive nucleotides thereof, and complementary sequences thereof.

In some aspects, the techniques described herein relate to a molecular marker for a Fusarium wilt resistance allele, including: at least one sequence selected from the group consisting of SEQ ID NO: 1, cDNA sequences thereof, fragments of at least 18 consecutive nucleotides thereof, and complementary sequences thereof. In some aspects, the molecular marker is selected from SEQ ID NOs: 22-24, and combinations thereof.

In some aspects, the techniques described herein relate to a molecular marker for distinguishing a plant having at least one Fusarium wilt resistance allele, including: at least one sequence selected from the group consisting of SEQ ID NO: 3, cDNA sequences thereof, fragments of at least 18 consecutive nucleotides thereof, and complementary sequences thereof. In some aspects, the molecular marker is selected from SEQ ID NOs: 32-34, and combinations thereof.

In some aspects, the techniques described herein relate to a recombinant nucleic acid molecule including a nucleotide sequence encoding SEQ ID NO: 2, or a sequence at least 90% identical thereto.

In some aspects, the techniques described herein relate to a recombinant nucleic acid molecule including a nucleotide sequence encoding SEQ ID NO: 4, or a sequence at least 90% identical thereto.

In some aspects, the disclosure relates to a vector including the recombinant nucleic acid molecules described herein.

In some aspects, the techniques described herein relate to a method for identifying a plant, plant part, or plant cell including a FUS1 allele, which confers resistance to Fusarium wilt when present in a strawberry plant, wherein said FUS1 allele encodes a TIR-NB-ARC-LRR protein that includes in its amino acid sequence a C-terminal Jelly-roll/Ig-like Domain, the method including: determining the presence of a genomic nucleotide sequence or a resistance-conferring part thereof in the genome of a plant, plant part, or plant cell, wherein said nucleotide sequence encodes a protein having at least 90% sequence similarity to SEQ ID NO: 2.

In some aspects, the techniques described herein relate to a method for identifying a plant, plant part, or plant cell including a FUS4 allele, which confers resistance to Fusarium wilt when present in a strawberry plant, wherein said FUS4 allele encodes a leucine rich receptor like kinase, said method including: determining the presence of genomic nucleotide sequence or a resistance-conferring part thereof in the genome of a plant, plant part, or plant cell, wherein said nucleotide sequence encodes a protein having at least 90% sequence similarity to SEQ ID NO: 4.

In some aspects, the techniques described herein relate to a molecular marker for a Fusarium wilt resistance allele, including: at least one sequence selected from the group consisting of SEQ ID NO: 28, cDNA sequences thereof, fragments of at least 18 consecutive nucleotides thereof, and complementary sequences thereof.

In some aspects, the techniques described herein relate to a molecular marker for distinguishing a plant having at least one Fusarium wilt resistance allele, including: at least one sequence selected from the group consisting of SEQ ID NO: 29, cDNA sequences thereof, fragments of at least 18 consecutive nucleotides thereof, and complementary sequences thereof.

In some aspects, the techniques described herein relate to a genetically engineered Fragaria spp. plant or plant part including a Fusarium wilt resistance allele designated FUS4, wherein said FUS4 allele encodes a protein including SEQ ID NO: 4, or a sequence at least 90% identical thereto, wherein the protein encoded by said allele is a leucine rich receptor like kinase, and wherein said genetically engineered Fragaria spp. plant or plant part has a Fusarium wilt resistance rAUDPC score of less than 0.04.

In some aspects, the techniques described herein relate to a genetically engineered Fragaria spp. plant or plant part including a Fusarium wilt resistance allele designated FUS1, wherein said FUS1 allele encodes a protein including SEQ ID NO: 2, or a sequence at least 90% identical thereto, wherein said FUS1 allele encodes a TIR-NB-ARC-LRR protein that includes in its amino acid sequence a C-terminal Jelly-roll/Ig-like Domain, and wherein said genetically engineered Fragaria spp. plant or plant part has an average Fusarium wilt resistance rAUDPC score of less than 0.04.

In some aspects, the genetically engineered Fragaria spp. plant or plant parts described herein are cisgenic, wherein the FUS1 and/or FUS4 allele is driven by its native promoter.

In some aspects, the genetically engineered Fragaria spp. plant or plant parts described herein are transgenic. In some aspects, the FUS1 and/or FUS4 allele is driven by a constitutive promoter. In some aspects, the genetically engineered Fragaria spp. plant or plant part has increased FUS1 and/or FUS4 expression compared to a non-transgenic Fragaria spp. plant or plant part. In some aspects, the increased expression of FUS1 and/or FUS4 is between 1.1 and 20-fold greater than a non-transgenic Fragaria spp. plant or plant part.

In addition to the exemplary aspects and embodiments described above, further aspects and embodiments will become apparent by the following descriptions.

### BRIEF DESCRIPTION OF THE DRAWINGS

FIG. 1A is a graphic of the genetic elements present on the T-DNA insert from pSIM6102

FIG. 1B is a graphic of the genetic elements present on the T-DNA insert from pSIM6103

FIG. 1C is a graphic of the genetic elements present on the T-DNA insert from pSIM6145.

FIG. 1D is a graphic of the genetic elements present on the T-DNA insert from pSIM6274.

FIG. 2 shows photographs of plants with various Fusarium wilt disease scoring.

FIG. 3 shows a partial protein alignment for three resistant FUS1 alleles: BG-4352_39087 (SEQ ID NO: 11), BG123258_58000 (SEQ ID NO: 2), and PS9271_PR.4.1.4.0 (SEQ ID NO: 11) against the susceptible alleles Inspire_2B-1 (SEQ ID NO: 7) and Fxa2Dg200066 (from ‘UCD Royal Royce’, SEQ ID NO: 9). The C-JID domain is indicated by the box.

FIG. 4 shows a partial protein alignment for the resistant alleles BG-4352_39087 (SEQ ID NO: 11) and BG123258_58000 (SEQ ID NO: 2) against susceptible alleles Inspire_2B-1 (SEQ ID NO: 7) and Fxa2Dg200066 (from ‘UCD Royal Royce’, SEQ ID NO: 9). The second and third leucine rich repeat (LRR) domains are indicated by the boxes.

FIG. 5 shows disease assay results for pSIM6103 numbered transformants with Inspire, the susceptible control, and PS 3.108, the resistant control.

FIG. 6 shows disease assay results for pSIM6102 numbered transformants with Inspire, the susceptible control, and PS 3.108, the resistant control.

FIG. 7 shows disease assay results for pSIM6145 numbered transformants with Inspire, the susceptible control, and PS 3.108, the resistant control.

FIG. 8 shows disease assay re-screening results for selected lines from pSIM6102, pSIM6103, and pSIM6145 with Inspire, the susceptible control, and PS 3.108, the resistant control.

FIG. 9 shows the average rAUDPC score by PS 3.108 normalized FUS1 expression in fusarium inoculated root tissue for all lines which underwent disease re-screening. Shapes indicate the identified FUS1 copy number.

FIG. 10 shows the average rAUDPC score by PS 3.108 normalized FUS1 expression in fusarium inoculated root tissue for all lines which underwent disease re-screening, excluding pSIM6103 lines. Shapes indicate the identified FUS1 copy number.

FIG. 11 shows the average rAUDPC score by PS 3.108 normalized FUS4 expression in fusarium inoculated root tissue for all pSIM6145 lines which underwent disease re-screening. Shapes indicate the identified FUS1 copy number.

### DETAILED DESCRIPTION

In the description and tables which follow, a number of terms are used. In order to provide a clear and consistent understanding of the specification and claims, including the scope to be given such terms, the following definitions are provided.

### Definitions

The term “a” or “an” refers to one or more of that entity; for example, “a primer” refers to one or more primers or at least one primer. As such, the terms “a” (or “an”), “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used in this application, the term “about” is meant to cover any normal fluctuations appreciated by one of ordinary skill in the relevant art. The term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used in this application, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Artificially manipulated, as used herein, “artificially manipulated” means to move, arrange, operate or control by the hands or by mechanical means or recombinant means, such as by genetic engineering techniques, a plant or plant cell, so as to produce a plant or plant cell that has a different biological, biochemical, morphological, or physiological phenotype and/or genotype in comparison to unmanipulated, naturally-occurring counterpart.

In the context of this disclosure, an “elite strawberry plant” is a non-naturally occurring plant having a genotype that results into an accumulation of distinguishable and desirable traits which is the result of human intervention, and is achieved by crossing and selection, mutagenizing, transforming or otherwise introducing such traits.

As used herein, “biomolecular characterization” refers to using molecular markers, including DNA, RNA, and proteins, to determine the genetic characteristics of cells or tissues.

Cisgenic as used herein refers to a plant that has been genetically modified with a natural gene from a crossable-sexually compatible plant. The cisgene includes the introns and is flanked by its native promoter and terminator in the normalsense orientation. Cisgenic plants therefore are similar to traditionally bred plants.

Degenerate primer. A “degenerate primer” is an oligonucleotide that contains sufficient nucleotide variations that it can accommodate base mismatches when hybridized to sequences of similar, but not exact, homology.

Event. Event refers to the unique DNA recombination event that took place in one plant cell, which was then used to generate an entire plant. Plant cells are transformed with a vector carrying a DNA insert of interest. Transformed cells are regenerated into plants, and each resulting plant represents a unique event. Different events possess differences in the number of copies of DNA insert in the cell genome, the arrangement of the DNA insert copies and/or the DNA insert location in the genome.

Foreign. “Foreign” with respect to a nucleic acid is non-native nucleic acid.

Functional homolog. In this disclosure, “functional homolog” refers to a protein or a gene (DNA sequence) encoding a protein that shares the same or similar functions in a biological, biochemical, catalytic or metabolic process. The “functional homolog” may be an actual homolog by virtue of descent from a common ancestor, or it may be unrelated (for example, an analogous sequence or protein arisen from convergent evolution).

Single locus Converted (Conversion). Single locus converted (conversion) plant refers to plants wherein essentially all of the desired morphological and physiological characteristics of a variety are recovered in addition to the one or more loci transferred into the variety via, for example, the backcrossing technique, via genetic engineering or via mutation.

Genetically engineered as used herein refers artificial genetic manipulation by a person. “Genetically engineered” thus includes, for example, cisgenics, transgenics, and targeted genome editing.

Genetic rearrangement. Refers to the re-association of genetic elements that can occur spontaneously in vivo as well as in vitro which introduce a new organization of genetic material. For instance, the splicing together of polynucleotides at different chromosomal loci, can occur spontaneously in vivo during both plant development and sexual recombination. Accordingly, recombination of genetic elements by non-natural genetic modification techniques in vitro is akin to recombination events that also can occur through sexual recombination in vivo.

Isolated. “Isolated” refers to any nucleic acid or compound that is physically separated from its normal, native environment. The isolated material may be maintained in a suitable solution containing, for instance, a solvent, a buffer, an ion, or other component, and may be in purified, or unpurified, form. Isolated may also refer to nucleic acid that has been isolated from an organism and is maintained, for example, in a plasmid.

Marketable Yield. As used herein, “marketable yield” refers to the total weight of USDA grade No. 1 and grade No. 2 fruit combined and may be presented as crates or flats per acre. Marketable yield is separate and distinct from total yield.

Native. A “native” genetic element refers to a nucleic acid that naturally exists in, originates from, or belongs to the genome of a plant that is to be transformed.

Non-natural nucleotide junction. “Non-natural nucleotide junction” or “non-naturally occurring nucleotide junction” refers to a sequence of nucleotides that do not occur in nature. Rather, these sequences are formed via a genetic transformation event. For example, the genetic transformation events described herein may be created with expression cassettes that contain no non-native DNA. Thus, these non-natural nucleotide junctions are composed of native nucleotides, but these nucleotides are in a genetic arrangement that does not occur in nature, but which results from the manipulation of man that occurs during the genetic transformation of the plant.

Operably linked. Combining two or more molecules in such a fashion that in combination they function properly in a plant cell. For instance, a promoter is operably linked to a structural gene when the promoter controls transcription of the structural gene.

Plant. As used herein, the term “plant” includes a monocot or a dicot. The word “plant,” as used herein, also encompasses plant cells, seed, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds and microspores.

Plant Parts. As used herein, the term “plant parts” (or a part thereof) includes but is not limited to protoplast, leaf, stem, root, root tip, anther, pistil, seed, embryo, pollen, ovule, cotyledon, hypocotyl, flower, tissue, petiole, cell, meristematic cell, and the like.

Progeny. As used herein, includes an F₁ strawberry plant produced from the cross of two strawberry plants where at least one plant includes the selected event or Fusarium resistance gene and progeny further includes, but is not limited to, subsequent F₂, F₃, F₄, F₅, F₆, F₇, F₈, F₉, generations.

Quantitative Trait Loci (QTL). Quantitative trait loci (QTL) refer to genetic loci that control to some degree numerically representable traits that are usually continuously distributed.

Recombinant. As used herein, refers to DNA, proteins, cells, or organisms that are man-made by combining genetic material from two different sources.

Regulatory sequences. Refers to those sequences which are standard and known to those in the art that may be included in the expression vectors to increase and/or maximize transcription of a gene of interest or translation of the resulting RNA in a plant system. These include, but are not limited to, promoters, peptide export signal sequences, introns, polyadenylation, and transcription termination sites.

Resistance-conferring as used herein means the part of the sequence that confers resistance. For example, a resistance-conferring part could be one or more genetic changes, such as a single nucleotide polymorphism.

rAUDPC as used herein refers to the relative area under the disease progress curve. This is calculated by dividing the area under the disease progress curve (AUDPC) by the AUDPC maximum potential (defined as the total number of days between the first and last rating day multiplied by 100). Disease ratings were determined as described in Table 6, example plants shown in FIG. 2.

Sequence identity. “sequence identity” or “identity” in the context of two nucleic acid or polypeptide sequences includes reference to the number of residues in the two sequences which are the same when aligned for maximum correspondence over a specified comparison window.

When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988). The comparison of sequences and determination of percent identity between two sequences can be accomplished using a mathematical algorithm. An example of a local alignment algorithm utilized for the comparison of sequences is the NCBI Basic Local Alignment Search Tool (BLAST®) (Altschul et al. 1990 J. Mol. Biol. 215: 403-10), which is available from several sources, including the National Center for Biotechnology Information (NCBI, Bethesda, Md.) and on the Internet, for use in connection with the sequence analysis programs blastp, blastn, blastx, tblastn and tblastx. It can be accessed on the internet via the National Library of Medicine (NLM)'s world-wide-web URL. A description of how to determine sequence identity using this program is available at the NLM's website on BLAST tutorial. Another example of a mathematical algorithm utilized for the global comparison of sequences is the Clustal W and Clustal X (Larkin et al. 2007 Bioinformatics, 23, 2947-294, Clustal W and Clustal X version 2.0) as well as Clustal omega. Unless otherwise stated, references to sequence identity used herein refer to the NCBI Basic Local Alignment Search Tool (BLAST®).

Total Yield. Total yield refers to the total weight of fruit (both marketable and unmarketable) and may be presented as crates or flats per acre.

Transformation of plant cells. A process by which DNA is integrated into the genome of a plant cell. The integration may be transient or stable. “Stably” refers to the permanent, or non-transient retention and/or expression of a polynucleotide in and by a cell genome. Thus, a stably integrated polynucleotide is one that is a fixture within a transformed cell genome and can be replicated and propagated through successive progeny of the cell or resultant transformed plant. Transformation may occur under natural or artificial conditions using various methods well known in the art. Transformation may rely on any known method for the insertion of nucleic acid sequences into a prokaryotic or eukaryotic host cell, including Agrobacterium-mediated transformation protocols, viral infection, whiskers, electroporation, heat shock, lipofection, polyethylene glycol treatment, micro-injection, and particle bombardment.

The term “resistant”, or “resistance” is used herein as defined by the International Seed Federation (ISF), a non-governmental, non-profit organization representing the seed industry. Resistance is defined as the ability of plant types to restrict the growth and development of a specified pest or pathogen and/or the damage they cause when compared to susceptible plant varieties under similar environmental conditions and pest or pathogen pressure. Resistant plant types may still exhibit some disease symptoms or damage, but they may still produce a crop, even though the plants may appear visually stunted and the marketable and/or total yield is reduced compared to uninfected plants. Two levels of resistance are defined. The term “high/standard resistance” is used for plant varieties that highly restrict the growth and development of the specified pest or pathogen under normal pest or pathogen pressure when compared to susceptible varieties. “Moderate/intermediate resistance” is applied to plant types that restrict the growth and development of the specified pest or pathogen, but exhibit a greater range of symptoms or damage compared to plant types with high resistance. Plant types with intermediate resistance will show less severe symptoms than susceptible plant varieties, when grown under similar field conditions and pathogen pressure. Methods of evaluating resistance are well known to one skilled in the art. Such evaluation may be performed by visual observation of a plant or a plant part (e.g., leaves, roots, flowers, fruits et. al) in determining the severity of symptoms and may be given a resistance score on a scale of 0 to 10 based on the severity of the reaction or symptoms, with 0 being the resistance score applied to the most resistant plants (e.g., no symptoms), and 10 the score applied to the plants with the most severe symptoms (death from the pathogen). In addition to such visual evaluations, disease evaluations can be performed by determining the pathogen bio-density in a plant or plant part using electron microscopy and/or through molecular biological methods, such as protein hybridization (e.g., ELISA, measuring pathogen protein density) and/or nucleic acid hybridization (e.g., RT-PCR, measuring pathogen RNA density). Depending on the particular pathogen/plant combination, a plant may be determined resistant to the pathogen, for example, if it has a pathogen RNA/DNA and/or protein density that is about 50%, or about 40%, or about 30%, or about 20%, or about 10%, or about 5%, or about 2%, or about 1%, or about 0.1%, or about 0.01%, or about 0.001%, or about 0.0001% of the RNA/DNA and/or protein density in a susceptible plant.

As used herein, the term “full resistance” is referred to as complete failure of the pathogen to develop after infection, and may either be the result of failure of the pathogen to enter the cell (no initial infection) or may be the result of failure of the pathogen to multiply in the cell and infect subsequent cells (no subliminal infection, no spread). The presence of full resistance may be determined by establishing the absence of pathogen protein or pathogen RNA in cells of the plant, as well as the absence of any disease symptoms in said plant, upon exposure of said plant to an infective dosage of pathogen (i.e. after ‘infection’). Among breeders, this phenotype is often referred to as “immune.” “Immunity” as used herein thus refers to a form of resistance characterized by absence of pathogen replication even when the pathogen is actively transferred into cells by e.g. electroporation.

As used herein, the term “tolerant” is used herein to indicate a phenotype of a plant wherein disease-symptoms remain absent upon exposure of said plant to an infective dosage of pathogen, whereby the presence of a systemic or local pathogen infection, pathogen multiplication, at least the presence of pathogen genomic sequences in cells of said plant and/or genomic integration thereof can be established. Tolerant plants are therefore symptomless carriers of the pathogen. Sometimes, pathogen sequences may be present or even multiply in plants without causing disease symptoms.

As used herein, the term “susceptible” is used herein to refer to a plant having no resistance to the pathogen resulting in entry of the pathogen into the plant and multiplication and systemic spread of the pathogen, resulting in disease symptoms. The term “susceptible” is therefore equivalent to “non-resistant”.

The term “single allele converted plant” as used herein refers to those plants wherein essentially all of the desired morphological and physiological characteristics of an inbred are present in addition to the single allele transferred into the inbred.

As used herein, the terms “introgression”, “introgressed” and “introgressing” refer to the process whereby genes of one species, variety or cultivar are moved into the genome of another species, variety or cultivar, by crossing those species. The crossing may be natural or artificial.

The process may optionally be completed by backcrossing to the recurrent parent, in which case introgression refers to infiltration of the genes of one species into the gene pool of another through repeated backcrossing of an interspecific hybrid with one of its parents. An introgression may also be described as a heterologous genetic material stably integrated in the genome of a recipient plant.

Transgenic plant. A genetically modified plant which contains at least one transgene.

Transgene. A transgene is a gene taken from one species, or artificially constructed (synthetic) and which is artificially introduced into the genome of another species.

Variant. A “variant,” as used herein, is understood to mean a nucleotide or amino acid sequence that deviates from the standard, or given, nucleotide or amino acid sequence of a particular gene or protein. The terms, “isoform,” “isotype,” and “analog” also refer to “variant” forms of a nucleotide or an amino acid sequence. An amino acid sequence that is altered by the addition, removal or substitution of one or more amino acids, or a change in nucleotide sequence, may be considered a “variant” sequence. The variant may have “conservative” changes, wherein a substituted amino acid has similar structural or chemical properties, e.g., replacement of leucine with isoleucine. A variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software, CLC Genomics Workbench (Germantown, MD) or EMBL-EBI online software.

### Overview

Two alleles conferring strawberry fusarium wilt resistance have been identified via bioinformatics approaches. The present disclosure relates to use of these sequences for marker-assisted breeding to introduce Fusarium wilt resistance via breeding methods. The disclosure also teaches methods for the precise introduction of resistance in any strawberry variety via genetic modification or gene editing approaches. The disclosure further relates to methods of identifying plants having one or more of the resistance alleles.

### Strawberry

Strawberry fruit is widely appreciated for its characteristic aroma, bright red color, juicy texture and sweetness. It is consumed in large quantities either fresh or in numerous prepared foods, including jams, juices, pies, milkshakes, etc.

Global strawberry production increased by 39.4% between 2008 and 2018. China led worldwide production at 3 million tons in 2018, followed by the United States with 1.3 million tons. Despite having the world's highest average yield/hectare, production in the United States over this time period did not show the sharp increases in production obtained in some other countries, including China. See, e.g., HortDaily, Sep. 23, 2020, Global strawberry production up by almost 40%, online publication.

Strawberry is a member of family Rosaceae and genus Fragaria. Garden strawberry (Fragaria x ananassa) is a widely grown hybrid species of the genus Fragaria, cultivated worldwide for their fruit. Cultivars of Fragaria x ananassa have replaced the woodland strawberry (Fragaria vesca) in large-scale commercial production fields. Pincot et al. (Oct. 1, 2020, Social Network Analysis of the Genealogy of Cultivated Strawberry: Retracing the Wild Roots of Heirloom and Modern Cultivars, Genetics Investigation, 31 pages) studied the pedigree records of 8,851 strawberry genotypes, including 2,656 cultivars developed since 1775. They identified 187 wild octoploid and 1,171 F. x ananassa founders in the genealogy they traced from the earliest hybrids to modern cultivars. They estimated that new cultivar selection cycle time over the past 200 years decreased from 16-17 years/generation to 6-10 years/generation.

Strawberry plants suffer from severe inbreeding depression, and most cultivars are highly heterozygous. Most cultivars are somewhat self-fertile, but good bee activity has been shown to improve pollination. In most cases, the flowers appear hermaphroditic in structure, but function as either male or female.

Strawberry plants are rosette-forming, herbaceous perennials. Strawberries occur in three basic flowering habits: short day, long day and day neutral. These classifications refer to the day length sensitivity of the plant and the type of photoperiod, which induces flower formation. Day neutral cultivars produce flowers regardless of the photoperiod. Most commercial strawberries are either short day or day neutral. Strawberry cultivars vary widely in size, color, flavor, shape, and degree of fertility, season of ripening, and susceptibility or resistance to pests.

The strawberry is not, from a botanical point of view, a berry. Technically, it is an aggregate accessory fruit, meaning that the fleshy part is derived not from the plant's ovaries but from the receptacle, that holds the ovaries. Each apparent “seed” (achene) on the outside of the fruit is actually one of the ovaries of the flower, with a seed inside it.

There are three main ways to propagate strawberry plants. The plants can be divided and transplanted once multiple crowns have been grown (or division of rhizomes), new plants can be grown from strawberry seeds, or the runners that strawberry plants put out can be controlled, guided and caused to root where clone plants can be utilized most efficiently. Attempting to grow strawberries from the seeds of commercial cultivars will likely not produce true-to-type plants. For purposes of commercial production, plants are propagated from runners and, in general, grown using annual plasticulture, as a perennial system of matted rows or mounds, or using compost socks. Greenhouse and indoor vertical farms produce small amounts of strawberries mostly during the off seasons. Naing et al. (2019, Plant Methods, 15:36) provide in vitro propagation methods for the production of morphologically and genetically stable plants of different strawberry cultivars using meristem cultures.

In strawberry breeding and farming, emphasis is placed on sugars, acids and volatile compounds, which improve the taste and fragrance of a ripe strawberry. Strawberries produce many important chemical organic compounds, including, for example, flavonoids, anthocyanins, fructose, glucose, malic acid and citric acid. Esters, terpenes and furans are chemical compounds having the strongest relationships to strawberry flavor and fragrance.

Edger et al. (2019, Origin and evolution of the octoploid strawberry genome. Nat. Genet. 51, 541-547) published the first chromosome-scale genome assembly for the octoploid strawberry sequence in February of 2019. The team obtained the 813.4 Mb cultivated commercial strawberry sequence from the California cultivar “Camarosa.” Modern strawberries have a complex octoploid genetics (i.e., 8 sets of chromosomes) and sequences for an estimated 7,096 genes (Bombarely et al., 2010, BMC Genomics, 11:503).

### Fusarium Wilt

Fusarium wilt is a vascular disease caused by the fungal pathogen Fusarium oxysporum. Fusarium oxysporum species complex is a diverse group of filamentous, ascomycetous fungi that are soil borne and able to cause disease in many economically important crop species. F. oxysporum has a wide host range, with specific strains that are genetically distinct and typically host specific. Fusarium oxysporum f. sp. Fragariae (aka Fusarium Fragariae) is the causal agent of Fusarium wilt of strawberry (aka yellows disease).

The disease was discovered on strawberries in California in 2006 (Koike et al., 2009, Plant Dis. 93(10): 1077) and has become a significant threat to production. Two races of F. oxysporum f. sp. Fragariae have been identified, with race 1 widely distributed in California, the largest production region in the U.S.

Fusarium wilt infection can lead to plant stunting, wilting, and vegetative necrosis, reducing yields and in some cases killing an entire strawberry bed or strawberry field.

Symptoms of the disease typically include slower growth than normal, stunting, wilting of foliage (particularly of older leaves), crown discoloration, drying and death of older leaves, and eventual plant death (Pastrana et al., 2019, Plant Disease, 103:1249-1251). The youngest leaves in the center of the plant often remain green and alive. Symptoms usually first appear well after plants are established. Plants bearing heavy fruit loads or subjected to stress often show the most severe symptoms. When internal tissues of plant crowns are examined, vascular and cortical tissues are dark to orange-brown. Internal tissues of the main roots are typically not discolored.

Henry et al. (2017, Plant Disease, 1010:550-556) found considerable diversity in their study of 59 isolates ofF. oxysporum f. sp. Fragariae obtained from diseased strawberry plants in California. They reported indications that horizontal gene transfer may have occurred. According to Gordon et al. (2015, California Strawberry Commission Annual Production Research Report, Pathology, U. of CA, 15-27), the California population of F. oxysporum f. sp. Fragariae is composed of three somatic compatibility groups (SCG 1, SCG 2 and SCG 3).

Pastrana et al. (2019) demonstrated that F. oxysporum f. sp. Fragariae could move through stolons of infected mother plants and colonize first-generation daughter plants. They also showed that the pathogen could move through stolons from first to second-generation daughter plants and that daughter plants of both generations were symptomless. Henry et al. (2019, Phytopathology, 109:770-779) showed the persistence of F. oxysporum f. sp. Fragariae in soil through asymptomatic colonization of rotation crops. Henry et al. (2020, Plant Pathology, 69(7):1218-1226) also showed that the pathogen can colonize organic matter in soil and persist through anaerobic soil disinfestation.

F. oxysporum f. sp. Fragariae is polyphyletic and limited genetic markers are available for its detection (Burkhardt et al., 2019, Plant Disease, 103:1006-1013).

### Strawberry Fusarium Wilt Resistance Genes

Two previous studies identified a major locus on chromosome 2 of strawberry (called the FW1 locus) contributing to resistance to race 1 (Pincot et al., 2018; Pincot et al., 2022). The FW1 locus was mapped to the tip of chromosome 2B, a near-telomeric haploblock spanning approximately 3.3 Mb. However, Pincot et al. 2022 could not ascertain if this location contained one, multi-allelic loci, or three paralogs, and the causative gene(s) were not identified.

### Identification of the FW1 Locus Genes—Targeted Genome Sequencing

Three resistant breeding lines, ‘BG-4.352’ (US PP26,921), ‘BG-12.3258’ (US PP35,166), and ‘PS-9271’ (also known as ‘Reliance’, US PP21,415) were grown in the greenhouse and genomic DNA was isolated from young leaf tissue using a modified CTAB protocol (Doyle and Doyle, 1987). Oxford Nanopore Technologies (ONT) libraries were prepared using SQK-LSK110 library kit (Oxford, UK) with the Long Fragment Buffer according to the ‘Ligation sequencing gDNA’ ONT protocol. Libraries were loaded onto a FLO-MIN106D R.9.4.1 flow cell attached to an ONT MinION (MK1b) device which was connected to an HP Z440 workstation equipped with a GeoForce RTX 3060 video card. MinKNOW (v22.05.5; Core v5.1.0) and Guppy (v6.1.5) were used to initiate sequencing runs with default parameters and the adaptive sampling option was used with the fast basecalling model to enrich for the region associated with resistance in F. vesca plus 60 kb buffer on either side. The ONT flow cell wash kit EXP-WSH004 (Oxford, UK) was used with the ONT ‘Flow Cell Wash Kit’ protocol to wash a flow cell and reload with a new library when most pores became unavailable or enough data had been collected for a line.

### Identification of the FW1 Locus Genes—Resistant Haplotype Identification

Raw data was basecalled on a local Linux server equipped with four Tesla V100 GPUs using Guppy (v6.3.2) (ONT, Oxford, UK) with the following parameters: --config dna_r9.4.1_450bps_sup.cfg --chunks_per_runner 320 --chunk_size 2000 --gpu_runners_per_device 4 --device cuda:all:100% --num_callers 20 --trim_strategy dna --trim_adapters --trim_barcodes --calib_detect. Read quality was assessed via the ‘ont_tutorial_basicqc’ pipeline (available on the world wide web at github.com/nanoporetech/ont_tutorial basicqc) and the R markdown file was modified to additionally output the N10 and N90 read statistics. After QC, basecalled reads were re-aligned to the F. vesca H4v4 (Edger et al., 2018), ‘UCD Royal Royce’ (US PP32,952) v1 (Hardigan et al., 2021), and ‘Inspire’ (US PP29,794) v2 (unpublished) genome assemblies via minimap2 (v2.24) (Li, 2018) with the following options: ‘-secondary=no -x map-ont -a’. Reads were mapped separately for each haplotype in ‘UCD Royal Royce’ and ‘Inspire’ and combined via Samtools (v1.14) (Li et al., 2009) merge. Read mappings to the associated resistance region in each assembly were extracted via Samtools (v1.14) (Li et al., 2009) view, read IDs from all assemblies were concatenated into a single file, and reads were subsetted from the fastq file via Seqtk (v1.3-r106) (https://github.com/lh3/seqtk) subseq, only retaining reads longer than 1 kb via Seqtk (v1.3-r106) seq. The subsetted fastq file was then used with Shasta (v0.10.0) (Shafin et al., 2020) and the following options: --config Nanopore-Phased-May2022 --Reads.minReadLength 5000. Contigs less than 1 kb were removed from the ‘Assembly-Phased.fasta’ file via Seqtk (v1.3-r106) seq. Bandage (v0.8.1) (Wick et al., 2015) was used to visualize the graphical fragment assembly file. ONT basecalled reads were aligned to the assembly via Minimap2 (v2.24) (Li, 2018) using the same parameters stated above and IGV (v2.13.0) (Robinson et al., 2011) was used to visualize read alignments. Marker flanking sequences were searched against the assembled haplotypes via BLASTN as implemented in Qiagen CLC Genomics Workbench (v22; Venlo, Netherlands) to identify which haplotype was associated with resistance by detecting the presence of the expected SNP.

### Identification of the FW1 Locus Genes—Transcriptome Sequencing

Bare-root plants from eight resistant breeding lines (including the three lines sequenced above with ONT) and ‘Inspire’ were dip-inoculated with a F. oxysporum f.sp. Fragariae race 1 isolate at a 1×10⁶ spore/ml concentration. Afterwards, plants were placed in a greenhouse. Root tissue was collected from inoculated and non-inoculated plants at 24 and 48 hours post inoculation (HPI), and immediately flash frozen in liquid nitrogen. Roots from three plants were pooled and designated as a replicate, and three replicates were collected per line, treatment, and time point. Total RNA was isolated using the Norgen Plant/Fungi Total RNA Purification Kit (Thorold, CA) and subsequently DNase treated with TURBO DNase (Invitrogen; Waltham, USA). Libraries were generated with the NEBNext Ultra II Directional Kit (Ipswich, USA) coupled with the IDT unique dual index barcodes (Newark, USA). Pooled libraries were sequenced on 1 Illumina NovaSeq S4 lane at the Texas A&M AgriLife Genomics and Bioinformatics Center to generate paired-end 150 nt reads.

### Identification of the FW1 Locus Genes—Allele Annotation and Differential Expression

Reads were quality checked and sequencing adapters removed using FastQC (v0.11.8) (https://www.bioinformatics.babraham.ac.uk/projects/fastqc/) and Cutadapt (v4.1) (Martin, 2011) with the following parameters: --nextseq-trim=20 --trim-n -m 80 -n 1. Reads were mapped separately to Fragaria vesca ‘Hawaii 4’ (v4) (Edger et al., 2018), ‘UCD Royal Royce’ (Hardigan et al., 2021), ‘Inspire’ (v2), and the three local ONT assemblies for ‘BG-4.352’, ‘BG-12.3258’, and ‘PS-9271’ with Hisat2 (v2.2.1) (Kim et al., 2019) with the following parameters: -k 16 --rna-strandness RF --max-intronlen 50000. Read alignments were imported into Qiagen CLC Genomics Workbench (v22; Venlo, Netherlands) and used as evidence to predict gene annotations for ‘BG-4.352’, ‘BG-12.3258’, and ‘PS-9271’ via the ‘Transcript Discovery’ tool. Annotated transcripts from F. vesca H4v4, ‘UCD Royal Royce’ v1, and ‘BG-12.3258’ were separately used with the trimmed reads in Kallisto (v0.48.0) (Bray et al., 2016) to quantify transcript abundances with the ‘quant’ function and the following parameters: --bias --rf-stranded. Abundances were subsequently processed by tximport (v1.26.0) (Soneson et al., 2015) and DESeq2 (v1.38.2) (Love et al., 2014) in R (v4.2.1) to obtain gene-level expression abundances and identify genes which were differentially expressed across all resistant lines compared to ‘Inspire’. Pearson's Correlation Coefficients (PCC) were calculated with the variance stabilized transformed counts and the following samples were removed from the dataset due to low PCC vales with other replicates (PCC <0.92): replicate 1 from PE-7.2054 24 HPI inoculated, replicate 3 from ‘BG-4.352’ 48 HPI inoculated, and replicate 1 from BG-4.367 24 HPI inoculated. Principal component analysis (PCA) was performed on the samples using the ‘plotPCA’ function. The likelihood ratio test was used in DESeq2 with the ‘Line’ factor removed in the reduced model, followed by retaining significant genes (adj. p-value<0.05) which had an absolute log 2-fold change >2 in all resistant lines compared to ‘Inspire’. Gene function was assessed via InterProScan (available on the world wide web at ebi.ac.uk/interpro/) to identify protein domains.

### Identification of the FW1 Locus Genes and Resistance Alleles—FUS1 and FUS4

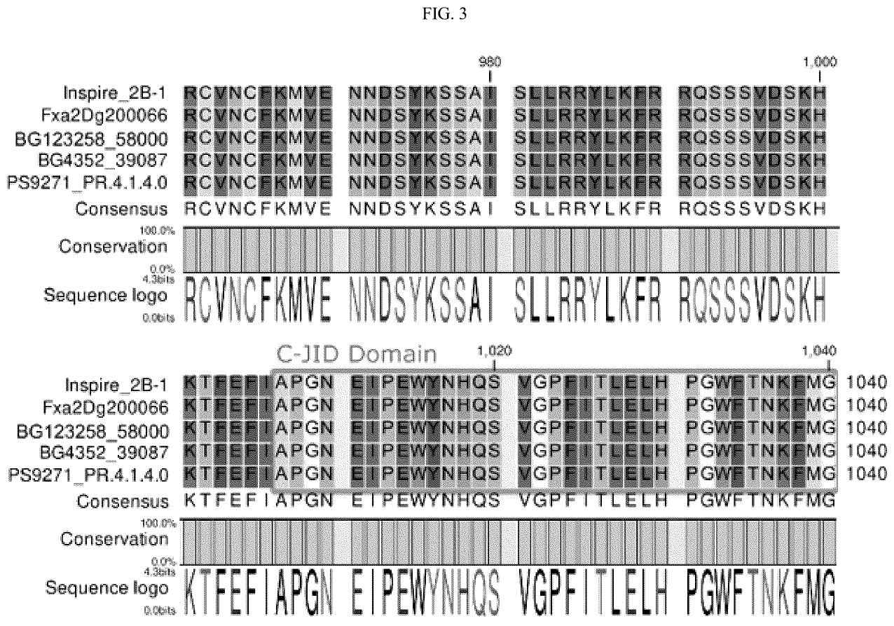

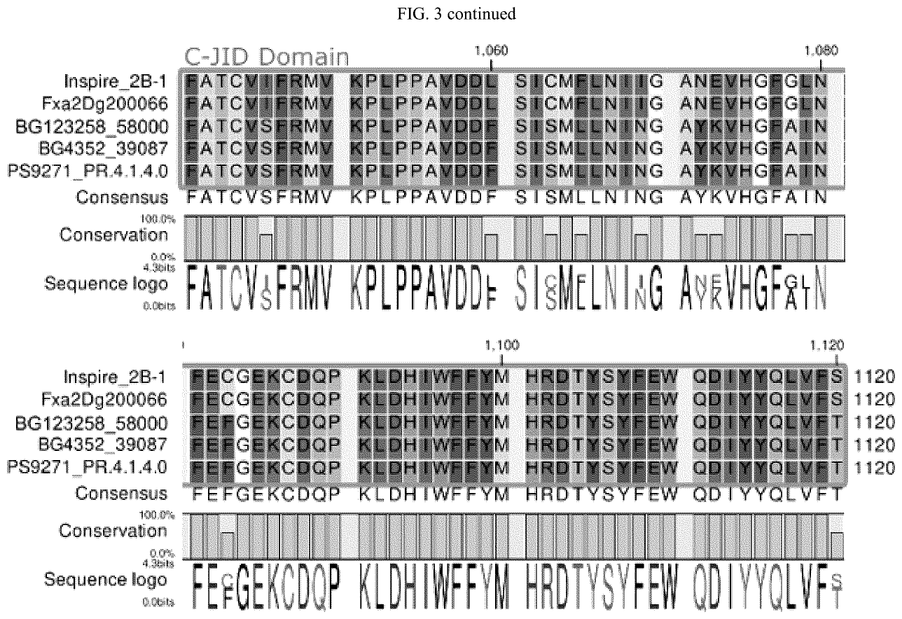

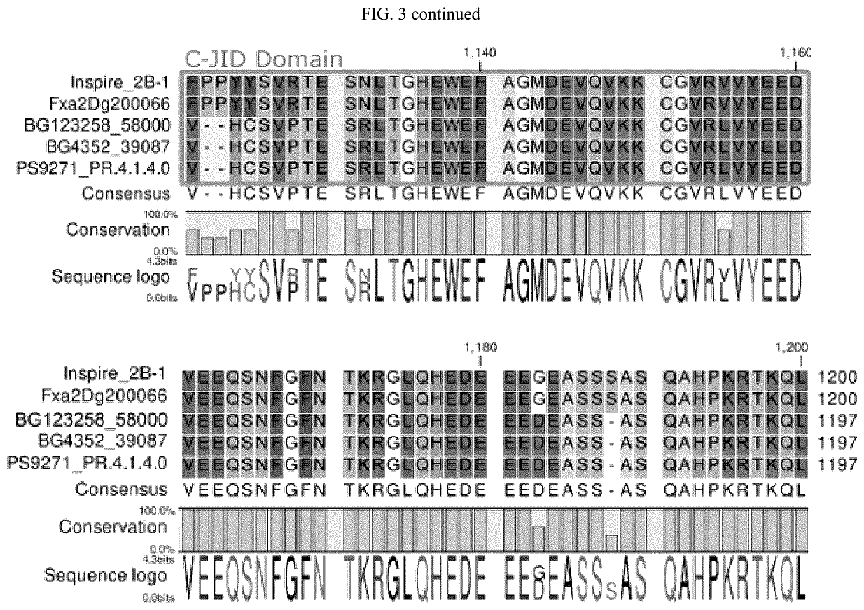

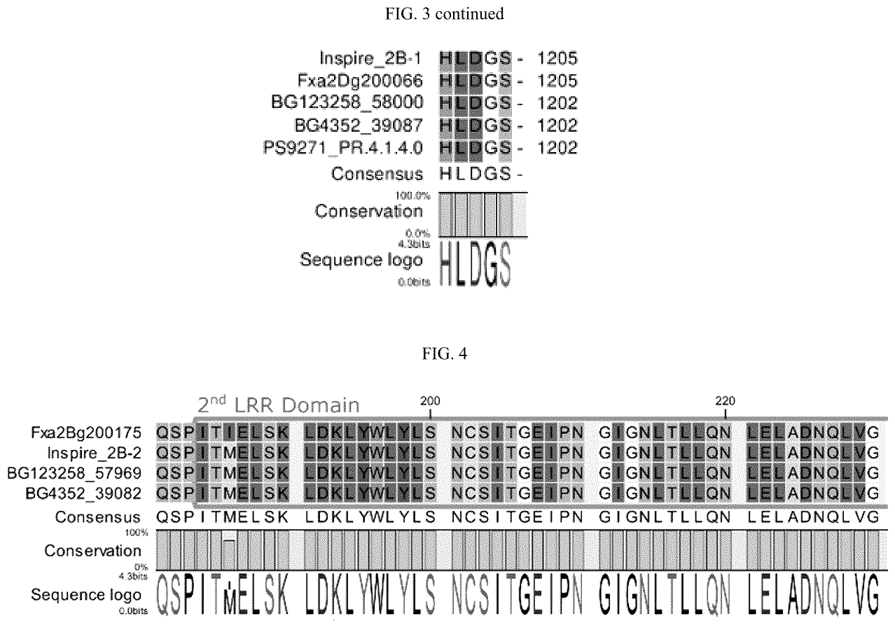

Five markers (Fof1 to Fof5) were identified as contributing to resistance and were located in the F. vesca (H4v4a2) genome (Edger et al., 2018; Li et al., 2019) on Chr02 from 1.0-2.6 Mb. Resistant haplotypes were identified for four of the markers in all three sequenced lines, with copy numbers ranging between 1 and 2 copies. Only two F. vesca genes were differentially expressed (average Transcripts Per Million (TPM)>1 and average Log-2 Fold Change (LFC)>2) in the Chr02 region with both having no known function. Several alleles on BG-12.3258 haplotypes associated with resistance (i.e. scaffolds containing the variant associated with resistance) were differentially expressed, including a TIR-NB-ARC-LRR (R gene) and a leucine rich receptor like kinase (RLK). In both cases, the differentially expressed allele was unique to resistant lines and all profiled resistant lines contained the allele. The R gene had very high similarity across both resistant and susceptible alleles throughout the sequence except for the C-JID (C-terminal Jelly-roll/Ig-like Domain) (84.9% ID in resistant allele) (FIG. 3), which is involved in effector recognition.

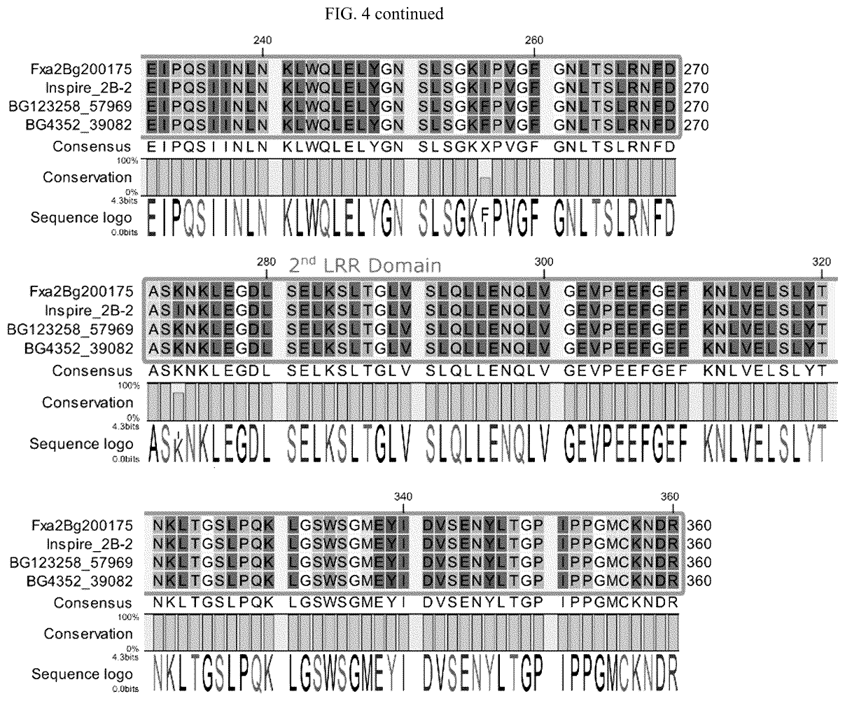

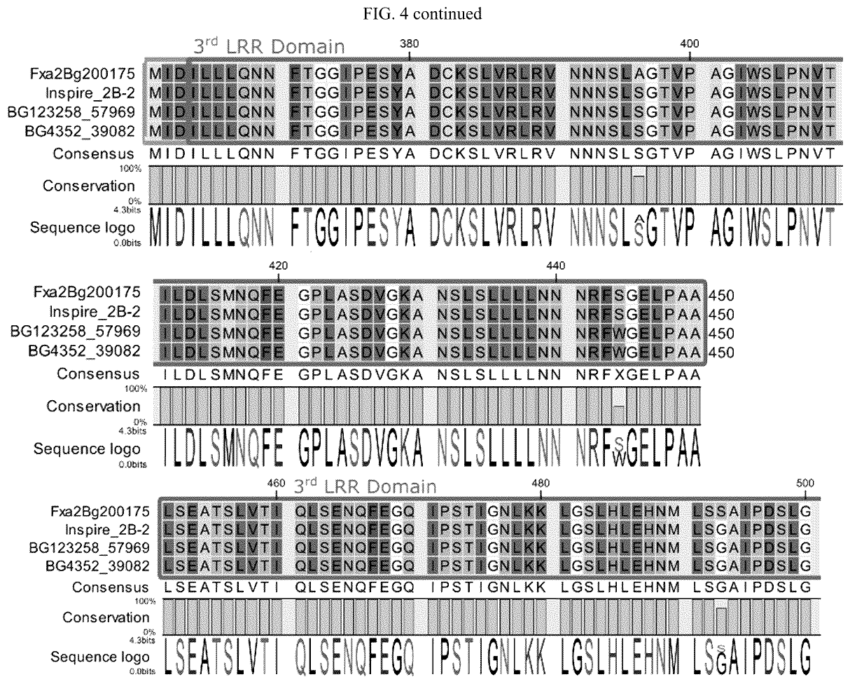

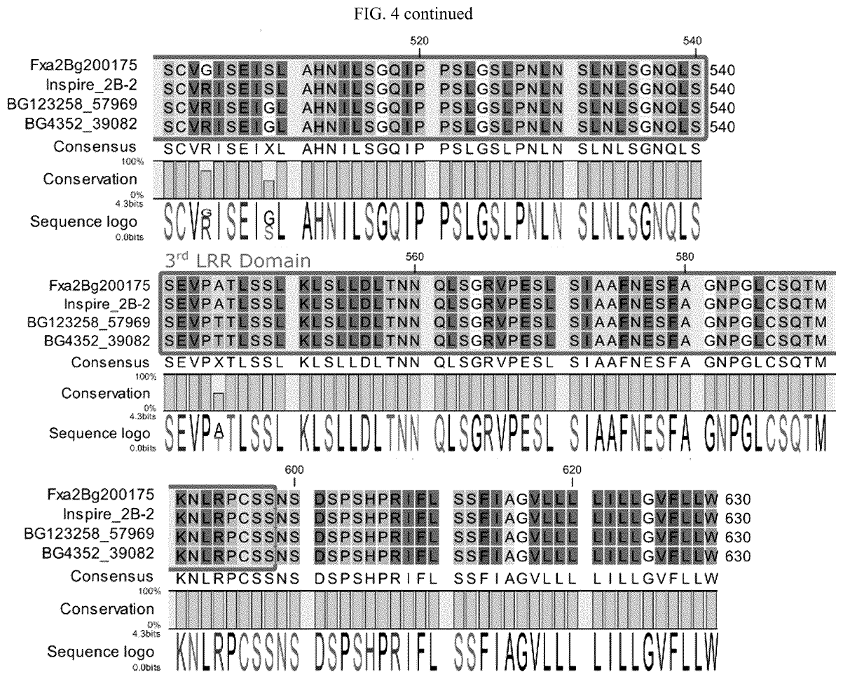

The RLK similarly had high sequence similarity between all alleles with only 4 amino acids unique to the resistant allele compared to the susceptible alleles (FIG. 4). One of these amino acid changes (S509G) is predicted to reside at the surface where the pathogen associated molecular pattern (PAMP) binds. Hereafter, the R gene is designated as FUS1 and the RLK is designated as FUS4.

### Identification of the FW1 Locus Genes—Construct Design and Cloning

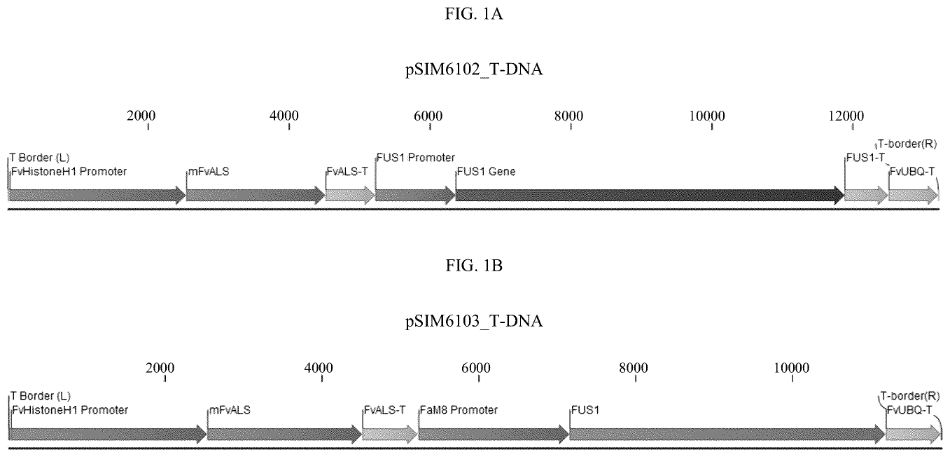

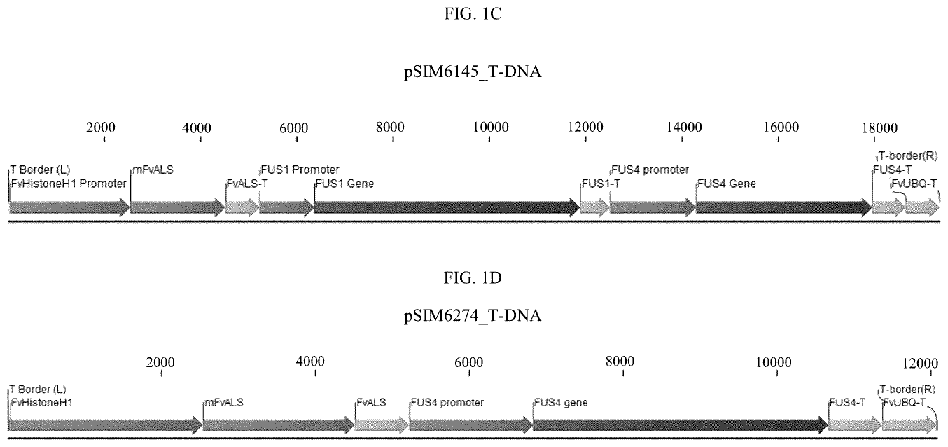

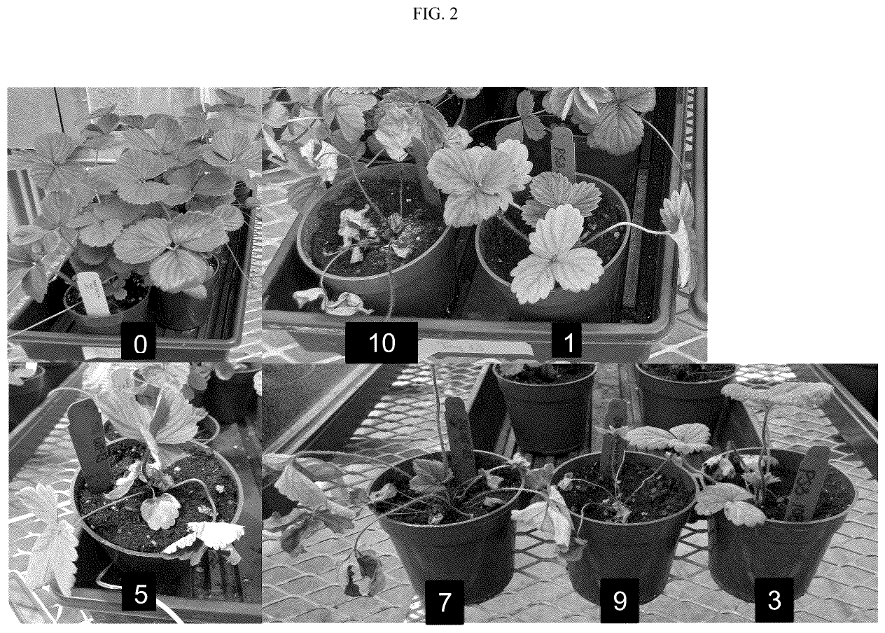

Four constructs were designed, including: pSIM6102 (FIG. 1A), pSIM6103 (FIG. 1B), pSIM6145 (FIG. 1C), and pSIM6274 (FIG. 1D). pSIM6102 contained the FUS1 gene driven by its native promoter and terminator (Table 1); pSIM6103 contained the FUS1 gene excluding untranslated regions driven by the F. x ananassa M8 constitutive promoter (Table 2); pSIM6145 contained FUS1 and FUS4 driven by their native promoters and terminators (Table 3); pSIM6274 contained the FUS4 gene driven its native promoter and terminator (Table 4). A total of 1,140 nt upstream ofthe transcriptional start site was used as the native promoter and 618 nt downstream of the transcriptional stop site was used the native terminator for FUS 1. A total of 1,787 nt upstream ofthe transcriptional start site was used as the native promoter and 696 nt downstream of the transcriptional stop site was used the native terminator for FUS4. FUS1 and FUS4 were isolated from genomic DNA from the resistant line ‘BG-4.352’ and inserted into the pENTR/D-TOPO vector (Invitrogen, Waltham, USA). To increase specificity, the sequences were initially amplified with AccuStart II Taq DNA polymerase (Quantaflio, Beverly, USA), followed by amplification with Q5 High Fidelity DNA polymerase (NEB, Newark, USA) using the first PCR product as template. Restriction enzyme digests followed by ligation with T4 DNA ligase (Promega, Madison, USA) were subsequently used to transfer the gene segments into the destination vector, which contained mutated. vesca acetolactate synthase (mFvALS) as the selection marker. All constructs were confirmed via Sanger sequencing and were subsequently transformed into Agrobacterium tumefaciens strain GV3101.

TABLE 1
pSIM6102 Genetic Elements of the T-DNA Insert

| | | Accession | | |
|---|---|---|---|---|
| Name | Origin | Number | Position (bp) | Intended Function |
| T Border (Left | pCambia- | AF234297 | 1 . . . 26 | Secondary cleavage site releases ssDNA |
| border site) | 1301 | | | insert from pSIM6102 (VanHaaren et al., |
| | | | | 1989) |
| Intervening | synthetic | na | 27 . . . 32 | Sequence used for DNA cloning |
| Sequence | | | | |
| FvHistoneH1 | F. vesca | na | 33 . . . 2,531 | Drives expression of mFvALS |
| Promoter | | | | |
| Intervening | synthetic | na | 2,532 . . . 2,537 | Sequence used for DNA cloning |
| Sequence | | | | |
| mFvALS | F. vesca | na | 2,538 . . . 4,508 | Confers resistance to Imazamox |
| Intervening | synthetic | na | 4,509 . . . 4,514 | Sequence used for DNA cloning |
| Sequence | | | | |
| FvALS | F. vesca | na | 4,515 . . . 5,214 | Terminates transcription of mFvALS |
| Terminator | | | | |
| Intervening | synthetic | na | 5,215 . . . 5,219 | Sequence used for DNA cloning |
| Sequence | | | | |
| FUS1 Promoter | F. ananassa | na | 5,220 . . . 6,359 | Drives expression of FUS1 |
| FUS1 gene | F. ananassa | na | 6,360 . . . 11,879 | FUS1 R gene |
| FUS1- | F. ananassa | na | 11,880 . . . 12,497 | Terminates transcription of FUS1 |
| Terminator | | | | |
| Intervening | synthetic | na | 12,498 . . . 12,504 | Sequence used for DNA cloning |
| Sequence | | | | |
| FvUBQ- | F. vesca | na | 12,505 . . . 13,204 | Terminates transcription |
| Terminator | | | | |
| T border (Right | pCambia- | AF234297 | 13,205 . . . 13,229 | Primary cleavage releases ssDNA insert |
| border site) | 1301 | | | from pSIM6102 (VanHaaren et al., |
| | | | | 1989) |

TABLE 2
pSIM6103 Genetic Elements of the T-DNA Insert

| | | Accession | | |
|---|---|---|---|---|
| Name | Origin | Number | Position (bp) | Intended Function |
| T Border (Left | pCambia- | AF234297 | 1 . . . 26 | Secondary cleavage site releases ssDNA |
| border site) | 1301 | | | insert from pSIM6103 (VanHaaren et al., |
| | | | | 1989) |
| Intervening | synthetic | na | 27 . . . 32 | Sequence used for DNA cloning |
| Sequence | | | | |
| FvHistoneH1 | F. vesca | na | 33 . . . 2,531 | Drives expression of mFvALS |
| Promoter | | | | |
| Intervening | synthetic | na | 2,532 . . . 2,537 | Sequence used for DNA cloning |
| Sequence | | | | |
| mFvALS | F. vesca | na | 2,538 . . . 4,508 | Confers resistance to Imazamox |
| Intervening | synthetic | na | 4,509 . . . 4,514 | Sequence used for DNA cloning |
| Sequence | | | | |
| FvALS | F. vesca | na | 4,515 . . . 5,214 | Terminates transcription of mFvALS |
| Terminator | | | | |
| Intervening | synthetic | na | 5,215 . . . 5,228 | Sequence used for DNA cloning |
| Sequence | | | | |
| FaM8 Promoter | F. ananassa | na | 5,229 . . . 7,149 | Drives expression of FUS1 |
| Intervening | synthetic | na | 7,150 . . . 7,155 | Sequence used for DNA cloning |
| Sequence | | | | |
| FUS1 | F. ananassa | na | 7,156 . . . 11,180 | FUS1 R gene without untranslated |
| | | | | regions |
| Intervening | synthetic | na | 11,181 . . . 11,186 | Sequence used for DNA cloning |
| Sequence | | | | |
| FvUBQ | F. vesca | na | 11,187 . . . 11,886 | Terminates transcription of FUS1 |
| Terminator | | | | |
| T border (Right | pCambia- | AF234297 | 11,887 . . . 11,911 | Primary cleavage releases ssDNA insert |
| border site) | 1301 | | | from pSIM6103 (VanHaaren et al., |
| | | | | 1989) |

TABLE 3
pSIM6145 Genetic Elements of the T-DNA Insert

| | | Accession | | |
|---|---|---|---|---|
| Name | Origin | Number | Position (bp) | Intended Function |
| T Border (Left | pCambia- | AF234297 | 1 . . . 26 | Secondary cleavage site releases |
| border site) | 1301 | | | ssDNA insert from pSIM6145 |
| | | | | (VanHaaren et al., 1989) |
| Intervening | synthetic | na | 27 . . . 32 | Sequence used for DNA cloning |
| Sequence | | | | |
| FvHistoneH1 | F. vesca | na | 33 . . . 2,531 | Drives expression of mFvALS |
| Promoter | | | | |
| Intervening | synthetic | na | 2,532 . . . 2,537 | Sequence used for DNA cloning |
| Sequence | | | | |
| mFvALS | F. vesca | na | 2,538 . . . 4,508 | Confers resistance to Imazamox |
| Intervening | synthetic | na | 4,509 . . . 4,514 | Sequence used for DNA cloning |
| Sequence | | | | |
| FvALS | F. vesca | na | 4,515 . . . 5,214 | Terminates transcription of mFvALS |
| Terminator | | | | |
| Intervening | synthetic | na | 5,215 . . . 5,219 | Sequence used for DNA cloning |
| Sequence | | | | |
| FUS1 Promoter | F. ananassa | na | 5,220 . . . 6,359 | Drives expression of FUS1 |
| FUS1 | F. ananassa | na | 6,360 . . . 11,879 | FUS1 R gene |
| FUS1 | F. ananassa | na | 11,880 . . . 12,497 | Terminates transcription of FUS1 |
| Terminator | | | | |
| Intervening | synthetic | na | 12,498 . . . 12,503 | Sequence used for DNA cloning |
| Sequence | | | | |
| FUS4 Promoter | F. ananassa | na | 12,504 . . . 14,290 | Drives expression of FUS4 |
| FUS4 | F. ananassa | na | 14,291 . . . 17,946 | FUS4 RLK gene |
| FUS4 | F. ananassa | na | 17,947 . . . 18,642 | Terminates transcription of FUS4 |
| Terminator | | | | |
| Intervening | synthetic | na | 18,643 . . . 18,648 | Sequence used for DNA cloning |
| Sequence | | | | |
| FvUBQ | F. vesca | na | 18,649 . . . 19,348 | Terminates transcription |
| Terminator | | | | |
| T border (Right | pCambia- | AF234297 | 19,349 . . . 19,373 | Primary cleavage releases ssDNA insert |
| border site) | 1301 | | | from pSIM6145 (VanHaaren et al., |
| | | | | 1989) |

TABLE 4
pSIM6274 Genetic Elements of the T-DNA Insert

| | | Accession | | |
|---|---|---|---|---|
| Name | Origin | Number | Position (bp) | Intended Function |
| T Border (Left | pCambia- | AF234297 | 1 . . . 26 | Secondary cleavage site releases |
| border site) | 1301 | | | ssDNA insert from pSIM6274 |
| | | | | (VanHaaren et al., 1989) |
| Intervening | synthetic | na | 27 . . . 32 | Sequence used for DNA cloning |
| Sequence | | | | |
| FvHistoneH1 | F. vesca | na | 33 . . . 2,531 | Drives expression of mFvALS |
| Promoter | | | | |
| Intervening | synthetic | na | 2,532 . . . 2,537 | Sequence used for DNA cloning |
| Sequence | | | | |
| mFvALS | F. vesca | na | 2,538 . . . 4,508 | Confers resistance to Imazamox |
| Intervening | synthetic | na | 4,509 . . . 4,514 | Sequence used for DNA cloning |
| Sequence | | | | |
| FvALS | F. vesca | na | 4,515 . . . 5,214 | Terminates transcription of mFvALS |
| Terminator | | | | |
| Intervening | synthetic | na | 5,215 . . . 5,220 | Sequence used for DNA cloning |
| Sequence | | | | |
| FUS4 Promoter | F. ananassa | na | 5,221 . . . 7,006 | Drives expression of FUS4 |
| FUS4 gene | F. ananassa | na | 7,007 . . . 10,664 | FUS4 RLK gene |
| FUS4- | F. ananassa | na | 10,665 . . . 11,361 | Terminates transcription of FUS4 |
| Terminator | | | | |
| Intervening | synthetic | na | 11,362 . . . 11,367 | Sequence used for DNA cloning |
| Sequence | | | | |
| FvUBQ- | F. vesca | na | 11,368 . . . 12,067 | Terminates transcription |
| Terminator | | | | |
| T border (Right | pCambia- | AF234297 | 12,068 . . . 12,092 | Primary cleavage releases ssDNA insert |
| border site) | 1301 | | | from pSIM6274 (VanHaaren et al., |
| | | | | 1989) |

### Identification of the FW1 Locus Genes—Transformation

‘Inspire’ plants were grown in Magenta boxes (Magenta™, Model GA-7, Millipore Sigma, St. Louis, MO) containing 75 mL of Fragaria ananassa rooting (FaR) medium (half strength Murashige & Skoog Basal Medium with Vitamins, 15 g/L sucrose, 6 g/L agar, pH 5.7). Plants were cultured in a Percival growth chamber (Percival Scientific, Inc., Model CU-36L4, Perry, IA) with a 16-hour photoperiod of white fluorescent light (115 μmoles m-2·s-1) at 24° C. for 6 weeks. Leaves were cut and placed onto a filter paper moistened with MS liquid medium (full strength Murashige & Skoog Basal Medium with Vitamins, 30 g/L sucrose, pH 5.7). The outer edges of each leaf were removed, and each leaf was cut into 3-5×3-5 mm rectangular explant sectors. Explants were then transferred onto pre-culture medium (TIA1 medium: full strength Murashige & Skoog Basal Medium with Vitamins, 30 g/L sucrose, 6 g/L agar, pH 5.7, 2 mg/L thidiazuron, 0.25 mg/L indole-3-acetic acid) with the abaxial surface in contact with the medium. Explants were cultured at 24° C. in the dark for 7 days.

After the pre-culture period, the explants were transferred to a sterile Petri plate and transformed with various constructs using standard techniques known in the art. Explants were cultured at 24° C. in the dark for 48 hours. After the co-culture period, explants were transferred to shoot regeneration medium (TIA1) supplemented with 300 mg/L Timentin, 1 mL/L Plant Preservative Mixture (PPM), and 0.02 mg/L imazamox for plant selection. These explants were cultured at 24° C. in the dark for 7 days. After 7 days, explants were then placed under white fluorescent light (115 μmoles m-2·s-1) with a 16-hr photoperiod for an additional 21 days. Explants were then subcultured onto the same shoot regeneration medium for up to an additional 28 days.

As 4-5 mm shoot clumps formed, they were cut from the calli and transferred to Magenta boxes containing FaR medium supplemented with 300 mg/L Timentin, 1 mL/L PPM, and 0.04 mg/L imazamox for plant selection. Shoot clumps were cultured with a 16-hour photoperiod at 24° C. for 28 days. After 28 days, a single shoot per shoot clump was transferred into FaR medium supplemented with 300 mg/L Timentin and 1 mL/L PPM and were cultured for an additional 28 days. Afterwards, DNA from putative transformants was screened using primers specific to the T-DNA (Table 5) to confirm its presence.

TABLE 5
Screening and qPCR Primers

| Construct/Gene | Primer Name | Primer Sequence (5′→3′) | SEQ ID NO: |
|---|---|---|---|
| pSIM6103 | JF179 | GCTGTTGTCTTCTCGATTACT | SEQ ID NO: 18 |
| pSIM6103 | JF180 | CAAGTTCATCCAAGCACCA | SEQ ID NO: 19 |
| pSIM6102 | HY4730 | GGCAAGTAGGTGTCAATGGAAAT | SEQ ID NO: 20 |
| pSIM6102 | HY4722 | CTCCACCTTCGCACTTTCTC | SEQ ID NO: 21 |
| FUS1 qPCR primer | JF183 | GTATTCACAGTCCACTGCTC | SEQ ID NO: 22 |
| FUS1 qPCR primer | JF184 | ACTCCCATTCATGACCTGTT | SEQ ID NO: 23 |
| FUS1 FAM probe | JR172v2-FAM | TGCCAACAGAAAGCCGCCT | SEQ ID NO: 24 |
| CHC1 qPCR primer | JF122 | ATGCGCTGATTGAATCTC | SEQ ID NO: 25 |
| CHC1 qPCR primer | JF124 | TGGCTTCCGGTTCAAATAG | SEQ ID NO: 26 |
| CHC1 HEX probe | JF150-HEX | AGCTGGAGTAGCGAGTCG | SEQ ID NO: 27 |
| pSIM6145 | JF193 | TTACATATCCGTGGAGGAGAT | SEQ ID NO: 30 |
| pSIM6145 | JF194 | GAGCGAGAGAAGGAGAACT | SEQ ID NO: 31 |
| FUS4 qPCR primer | JF240 | GGGAACTTGAAGAAATTAGGTAG | SEQ ID NO: 32 |
| FUS4 qPCR primer | JF242 | AATGTTATGGGCAAGGCC | SEQ ID NO: 33 |
| FUS4 FAM probe | JF241-FAM | GGTGCCATACCAGACTCACTAGGT | SEQ ID NO: 34 |
| FvHistoneH1- | RF1258 | AACCCTAACCCTAATTCC | SEQ ID NO: 35 |
| mFvALS primer | | | |
| FvHistoneH1- | RF1259 | GAGAGGTTTTGGAGAAGG | SEQ ID NO: 36 |
| mFvALS primer | | | |
| FvHistoneH1- | RF1260-FAM | AAGGATCTATGGCGGCCACC | SEQ ID NO: 37 |
| mFvALS FAM probe | | | |
| AGP primer | RF1132 | TGCCACGTGTTTAAGGACT | SEQ ID NO: 38 |
| AGP primer | RF1133 | CCCGAAAACAAACAAATGAGAC | SEQ ID NO: 39 |
| AGP HEX probe | RF1173-HEX | AACACCACGAGGGCCCCACT | SEQ ID NO: 40 |

### Identification of the FW 1 Locus Genes—Copy Number Assays

To determine the number of T-DNA insertions, droplet digital PCR (ddPCR) was used with genomic DNA, ddPCR Supermix (Bio-Rad, Hercules, USA), restriction enzyme, and the primers and probes indicated in Table 5. ADP-Glucose Pyrophosphorylase (AGP) was used as the reference. Droplets were generated with a Bio-Rad QX200 Droplet Generator (Hercules, USA) and subsequently run on a Bio-Rad C1000 Thermal Cycler (Hercules, USA) with the following program: 95° C. for 10 min followed by 44 cycles of 94° C. for 30 s, 58° C. or 56° C. for 30 s, and 60° C. for 30 s followed by a temperature of 98° C. for 10 min. The FUS1 and FvHistoneH1-mFvALS primers used an annealing temperature of 58° C. and 56° C., respectively. Afterwards, droplets were read with a Bio-Rad QX200 Droplet Reader (Hercules, USA) and data were analyzed with Bio-Rad QuantaSoft software (Hercules, USA).

Identification of the FW1 Locus Genes—qPCR Assays

To quantify the expression level of FUS1 and FUS4, root tissue was collected from inoculated and non-inoculated plants at 24 hours post inoculation (HPI), and immediately flash frozen in liquid nitrogen. Roots from a single plant were designated as a replicate, and two replicates were collected per line and treatment. Total RNA was isolated using the Norgen Plant/Fungi Total RNA Purification Kit (Thorold, CA) and subsequently DNase treated with TURBO DNase (Invitrogen; Waltham, USA). DNase-treated RNA was reverse transcribed using SuperScript III first strand synthesis system (Invitrogen, Waltham, USA) followed by qPCR with AccuStart II Taq DNA polymerase (QuantaBio, Beverly, USA) with the primers and probes indicated in Table 5. Clathrin heavy chain 1 (CHC1) was used as the reference gene. FUS1 and FUS4 primers and probes were designed to specifically amplify the resistant allele. Samples were run on a Bio-Rad CFX96 Real-Time system (Hercules, USA) using the following program: 95° C. for 2 min followed by 40 cycles of 95° C. for 15 s, 58° C. for 15 s, and 72° C. for 30 s. Data were analyzed using Bio-Rad CFX Manager software (Hercules, USA). Replicates were averaged and values normalized to PS 3.108 expression. To quantify the abundance of F. oxysproum f.sp. Fragariae, previously designed primers and probes (Burkhardt et al., 2018) were used on isolated DNA.

### Identification of the FW1 Locus Genes—Disease Assays

Tissue culture plants were transplanted into Sunshine Mix #1 soil (Sun Gro Horticulture, Agawam, USA) and grown at 20° C./16° C. day/night temperature in a growth chamber. After six weeks, plant roots and crown tissue were dip inoculated for 10 min with the F. oxysporum f.sp. Fragariae race 1 isolate 157 (collected in Ventura County, CA, USA in 2016) at 1×10⁶ cfu/ml concentration. The inoculum was prepared by dislodging conidia from plates with 100 ml of autoclave distilled water and a sterile cell spreader, filtering through a double layer of cheesecloth to remove hyphae, and diluting with 0.1% water agar to the final concentration. After inoculation, plants were transferred to 4-inch pots, covered with plastic domes, and grown at 28° C./20° C. day/night temperatures and 16 hour photoperiod in the greenhouse. Plants were monitored weekly for symptoms and rated on a 0 to 10 scale (Table 6). Photographs of plants with various disease scoring is shown in FIG. 2. Disease scores were plotted by time and the resulting plot is called the disease progress curve. The relative area under the disease progress curve (rAUDPC) was calculated by dividing the area under the disease progress curve (AUDPC) by the AUDPC maximum potential (defined as the total number of days between the first and last rating day multiplied by 100).

TABLE 6
Strawberry Fusarium Disease Scoring

| Score | Description of plant |
|---|---|
| 0 | Healthy plant with erect growth and full vigor |
| 1 | Healthy plant with a small canopy and moderate vigor |
| 3 | Plant with some slight wilt, lower leaves affected |
| 5 | Plant with moderate wilt, with mature leaves collapsed but young leaves still |
| | healthy |
| 7 | Plant with severe wilt, most of the plant is has collapsed and is desiccated |
| 9 | Plant with severe wilt, the entire plant has collapsed and is desiccated |
| 10 | Dead plant |

### Methods for Identifying Plants Comprising the FUS1 or FUS4 Allele

In some embodiments, the disclosure teaches a method for identifying a plant, plant part, or plant cell comprising a FUS1 allele, which confers resistance to Fusarium wilt when present in a strawberry plant, wherein said FUS1 allele encodes a TIR-NB-ARC-LRR protein that comprises in its amino acid sequence a C-terminal Jelly-roll/Ig-like Domain, said method comprising: determining the presence of a genomic nucleotide sequence or a resistance-conferring part thereof in the genome of a plant, plant part, or plant cell, wherein said nucleotide sequence encodes a protein having at least 90% sequence similarity to SEQ ID NO: 2.

In some embodiments, the disclosure teaches a method for identifying a plant comprising the FUS1 allele comprising determining the presence of a coding sequence, or a resistance-conferring part thereof in the genome of a plant, wherein said sequence has at least 95%, sequence similarity to SEQ ID NO: 2. In some embodiments, the FUS1 allele encodes an amino acid sequence having at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 2.

In some embodiments, the nucleotide sequence comprises SEQ ID NO: 1 or a sequence at least 75% identical thereto. In some embodiments, the nucleotide sequence comprises SEQ ID NO: 1 or a sequence at least 85% identical thereto.

In some embodiments, the FUS1 allele comprises a sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 1.

In some embodiments, the nucleotide sequence comprises SEQ ID NO: 28 or a sequence at least 75% identical thereto. In some embodiments, the nucleotide sequence comprises SEQ ID NO: 28 or a sequence at least 85% identical thereto.

In some embodiments, the FUS1 allele comprises a sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 28.

In some embodiments, the disclosure teaches a method for identifying a plant, plant part, or plant cell comprising a FUS4 allele, which confers resistance to Fusarium wilt when present in a strawberry plant, wherein said FUS4 allele encodes a leucine rich receptor like kinase, said method comprising: determining the presence of genomic nucleotide sequence or a resistance-conferring part thereof in the genome of a plant, plant part, or plant cell, wherein said nucleotide sequence encodes a protein having at least 90% sequence similarity to SEQ ID NO: 4.

In some embodiments, the disclosure teaches a method for identifying a plant comprising the FUS4 allele comprising determining the presence of a coding sequence, or a resistance-conferring part thereof in the genome of a plant, wherein said sequence has at least 95%, sequence similarity to SEQ ID NO: 4. In some embodiments, the FUS4 allele encodes an amino acid sequence having at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 4.

In some embodiments, the nucleotide sequence comprises SEQ ID NO: 3 or a sequence at least 75% identical thereto. In some embodiments, the nucleotide sequence comprises SEQ ID NO: 3 or a sequence at least 85% identical thereto.

In some embodiments, the FUS4 allele comprises a sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 3.

In some embodiments, the nucleotide sequence comprises SEQ ID NO: 29 or a sequence at least 75% identical thereto. In some embodiments, the nucleotide sequence comprises SEQ ID NO: 29 or a sequence at least 85% identical thereto.

In some embodiments, the FUS4 allele comprises a sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 29.

In some embodiments, the method for determining the presence of a genomic nucleotide sequence or a resistance-conferring part thereof comprises biomolecular characterization.

Determining the presence of a genomic nucleotide sequence or a resistance-conferring part thereof in the genome of a plant, plant part, or plant cell strawberry plant may be performed using any suitable molecular biological method known in the art, including but not limited to (genomic) PCR amplification followed by Sanger sequencing, whole-genome-sequencing, transcriptome sequencing, sequence-specific target capture followed by next-generation sequencing (using, for example, the xGen® target capture system of Integrated DNA Technologies), specific amplification of LRR-domain-which may comprise gene sequences (using, for example, the Resistance Gene Enrichment Sequencing (RenSeq) methodology, as described in Jupe et al., 2013, Plant J. 76: 530-544) followed by sequencing, etcetera.

In some embodiments, determining the presence of a genomic nucleotide sequence or a resistance-conferring part thereof in the genome of a plant, plant part, or plant cell comprises restriction fragment length polymorphism, amplified fragment length polymorphism, random amplified polymorphic DNA, cleaved amplified polymorphic sequences, simple sequence repeat length polymorphism, single strand conformational polymorphism, heteroduplex analysis, single nucleotide polymorphism, expressed sequence tags, or sequence tagged sites.

### Marker-Assisted Breeding to Introduce Fusarium Wilt Resistance

In some embodiments, the disclosure teaches a method of producing a Fragaria spp. plant comprising a Fusarium wilt resistance allele, comprising: screening a Fragaria spp. plant for the presence of a Fusarium wilt resistance allele designated FUS1, wherein said FUS1 allele encodes a protein comprising SEQ ID NO: 2 or a sequence at least 90% identical thereto, and wherein the protein is a TIR-NB-ARC-LRR protein that comprises a C-terminal Jelly-roll/Ig-like Domain, and selecting a Fragaria spp. plant comprising said FUS1 allele; crossing the selected Fragaria spp. plant comprising said FUS1 allele with a second Fragaria spp. plant to produce progeny Fragaria spp. plants; screening said progeny Fragaria spp. plants for the presence of said FUS1 allele, and selecting a progeny Fragaria spp. plant comprising at least one copy of said FUS1 allele.

In some embodiments, the selected progeny Fragaria spp. plant comprising at least one copy of the FUS1 allele is resistant to Fusarium oxysporum f. sp. Fragariae.

In some embodiments, the selected Fragaria spp. plant and/or the second Fragaria spp. plant are Fragaria x ananassa. In some embodiments, the method of producing a Fragaria spp. plant comprising at least one copy of the FUS1 allele further comprises asexual propagation, outcrossing, or backcrossing the selected progeny Fragaria spp. plant.

In some embodiments, the selected progeny Fragaria spp. plant comprising at least one copy of the FUS1 allele comprises a sequence at least 75%, at least 76%, at least 77%, at least 78%, at least 79%, 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 1.

In some embodiments, the selected progeny Fragaria spp. plant comprising at least one copy of the FUS1 allele comprises a sequence at least 75%, at least 76%, at least 77%, at least 78%, at least 79%, 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 28.

In some embodiments, the selected progeny Fragaria spp. plant comprising at least one copy of the FUS1 allele encodes an amino acid sequence having at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 2.

In some embodiments, the FUS1 allele encoding a protein having at least 90% identity SEQ ID NO: 2 comprises a C-JID sequence having a serine at a position corresponding to 1,046 of SEQ ID NO: 2; a phenylalanine at a position corresponding to 1,060 of SEQ ID NO: 2; a serine at a position corresponding to 1,063 of SEQ ID NO: 2; a leucine at a position corresponding to 1,065 of SEQ ID NO: 2; an asparagine at a position corresponding to 1,069 of SEQ ID NO: 2; a tyrosine at a position corresponding to 1,072 of SEQ ID NO: 2; a lysine at a position corresponding to 1,073 of SEQ ID NO: 2; a phenylalanine at a position corresponding to 1,083 of SEQ ID NO: 2; a valine at a position corresponding to 1,121 of SEQ ID NO: 2; a histidine at a position corresponding to 1,122 of SEQ ID NO: 2; a cysteine at a position corresponding to 1,123 of SEQ ID NO: 2; a proline at a position corresponding to 1,126 of SEQ ID NO: 2; and an arginine at a position corresponding to 1,130 of SEQ ID NO: 2. In some embodiments, the FUS1 allele encoding a protein having at least 90% identity SEQ ID NO: 2 further includes an alanine at a position corresponding to 1,078 of SEQ ID NO:2; an isoleucine at a position corresponding to 1,079 of SEQ ID NO:2; a threonine at a position corresponding to 1,120 of SEQ ID NO: 2; and a leucine at a position corresponding to 1,153 of SEQ ID NO: 2.

In some embodiments, the screening a Fragaria spp. plant for the presence of said FUS1 allele comprises marker-assisted selection. In some aspects, the marker-assisted selection comprises a DNA marker selected from the group consisting of SEQ ID NO: 1, cDNA sequences thereof, fragments of at least 18 consecutive nucleotides thereof, and complementary sequences thereof. In some aspects, the marker-assisted selection comprises a DNA marker selected from the group consisting of SEQ ID NO: 28, cDNA sequences thereof, fragments of at least 18 consecutive nucleotides thereof, and complementary sequences thereof.

In some embodiments, the FUS1 allele having at least 75% identity to SEQ ID NO: 1 or encoding a protein having at least 90% identity SEQ ID NO: 2 comprises SEQ ID NO: 5.

In some embodiments, the screening the Fragaria spp. plant or the progeny Fragaria spp. plants for said FUS1 allele comprises identifying any one of the following amino acid changes: a serine at a position corresponding to 1,046 of SEQ ID NO: 2; a phenylalanine at a position corresponding to 1,060 of SEQ ID NO: 2; a serine at a position corresponding to 1,063 of SEQ ID NO: 2; a leucine at a position corresponding to 1,065 of SEQ ID NO: 2; an asparagine at a position corresponding to 1,069 of SEQ ID NO: 2; a tyrosine at a position corresponding to 1,072 of SEQ ID NO: 2; a lysine at a position corresponding to 1,073 of SEQ ID NO: 2; a phenylalanine at a position corresponding to 1,083 of SEQ ID NO: 2; a valine at a position corresponding to 1,121 of SEQ ID NO: 2; a histidine at a position corresponding to 1,122 of SEQ ID NO: 2; a cysteine at a position corresponding to 1,123 of SEQ ID NO: 2; a proline at a position corresponding to 1,126 of SEQ ID NO: 2; or an arginine at a position corresponding to 1,130 of SEQ ID NO: 2.

In some embodiments, the screening the Fragaria spp. plant or the progeny Fragaria spp. plants for said FUS1 allele comprises identifying an alanine at a position corresponding to 1,078 of SEQ ID NO:2; an isoleucine at a position corresponding to 1,079 of SEQ ID NO:2; a threonine at a position corresponding to 1,120 of SEQ ID NO: 2; and a leucine at a position corresponding to 1,153 of SEQ ID NO: 2.

In some embodiments, the screening the Fragaria spp. plant or the progeny Fragaria spp. plants for said FUS1 allele comprises identifying a double proline deletion at a position corresponding to 1, 122-1,123 of SEQ ID NO: 7.

In some embodiments, the molecular marker is selected from SEQ ID NOs: 22-24, and combinations thereof.

In some embodiments, the disclosure teaches a method of producing a Fragaria spp. plant comprising a Fusarium wilt resistance allele, comprising: screening a Fragaria spp. plant for the presence of a Fusarium wilt resistance allele designated FUS4, wherein said FUS4 allele encodes a protein comprising SEQ ID NO: 4, or a sequence at least 90% identical thereto, and wherein the protein encoded by said allele is a leucine rich receptor like kinase, and selecting a Fragaria spp. plant comprising said FUS4 allele; crossing the selected Fragaria spp. plant comprising said FUS4 allele with a second Fragaria spp. plant to produce progeny Fragaria spp. plants; screening said progeny Fragaria spp. plants for the presence of said FUS4 allele, and selecting a progeny Fragaria spp. plant comprising at least one copy of said FUS4 allele.

In some embodiments, the selected progeny Fragaria spp. plant comprising at least one copy of the FUS4 allele is resistant to Fusarium oxysporum f. sp. Fragariae.

In some embodiments, the selected Fragaria spp. plant and/or the second Fragaria spp. plant are Fragaria x ananassa. In some embodiments, the method of producing a Fragaria spp. plant comprising at least one copy of the FUS4 allele further comprises asexual propagation, outcrossing, or backcrossing the selected progeny Fragaria spp. plant.

In some embodiments, the selected progeny Fragaria spp. plant comprising at least one copy of the FUS4 allele comprises a sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 3.

In some embodiments, the selected progeny Fragaria spp. plant comprising at least one copy of the FUS4 allele comprises a sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 29.

In some embodiments, the selected progeny Fragaria spp. plant comprising at least one copy of the FUS4 allele encodes an amino acid sequence having at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 4.

In some embodiments, the FUS4 allele having at least 90% identity to SEQ ID NO: 4 comprises a phenylalanine at a position corresponding to 256 of SEQ ID NO: 4; a tryptophan at a position corresponding to 444 of SEQ ID NO: 4; a glycine at a position corresponding to 509 of SEQ ID NO: 4; and a threonine at a position corresponding to 545 of SEQ ID NO: 4.

In some embodiments, the screening a Fragaria spp. plant for the presence of said FUS4 allele comprises marker-assisted selection. In some aspects, the marker-assisted selection comprises a DNA marker selected from the group consisting of SEQ ID NO: 3, cDNA sequences thereof, fragments of at least 18 consecutive nucleotides thereof, and complementary sequences thereof. In some aspects, the marker-assisted selection comprises a DNA marker selected from the group consisting of SEQ ID NO: 29, cDNA sequences thereof, fragments of at least 18 consecutive nucleotides thereof, and complementary sequences thereof.

In some embodiments, the screening the Fragaria spp. plant or the progeny Fragaria spp. plants comprises identifying any one of the following amino acid changes: a phenylalanine at a position corresponding to 256 of SEQ ID NO: 4; a tryptophan at a position corresponding to 444 of SEQ ID NO: 4; a glycine at a position corresponding to 509 of SEQ ID NO: 4; and a threonine at a position corresponding to 545 of SEQ ID NO: 4.

In some embodiments, the molecular marker is selected from SEQ ID NOs: 32-34, and combinations thereof.

In some embodiments, the screening of a Fragaria spp. plant for the presence of a Fusarium wilt resistance allele comprises marker-assisted selection. Marker-assisted selection, or marker-assisted breeding, combines the use traditional plant breeding with molecular markers. Molecular markers in this context, is any identifier of a particular aspect of phenotype and/or genotype, and its inheritance can easily be followed from generation to generation.

In some embodiments, the screening a Fragaria spp. plant or progeny plant for the presence of a Fusarium wilt resistance gene comprises a DNA amplification-based method.

In some embodiments, the screening a Fragaria spp. plant or progeny plant for the presence of a Fusarium wilt resistance gene comprises restriction fragment length polymorphism, amplified fragment length polymorphism, random amplified polymorphic DNA, cleaved amplified polymorphic sequences, simple sequence repeat length polymorphism, single strand conformational polymorphism, heteroduplex analysis, single nucleotide polymorphism, expressed sequence tags, or sequence tagged sites.

### Engineering Fusarium Wilt Resistance Via Genetic Modification

In some embodiments, the disclosure teaches a method for conferring Fusarium resistance in a plant, comprising at least one of: (a) introducing a nucleic acid into a plant, plant, plant part, or plant cell, wherein said nucleic acid encodes an amino acid sequence at least 90% identical to SEQ ID NO: 2, and (b) introducing a nucleic acid into a plant, plant part, or plant cell, wherein said nucleic acid encodes an amino acid sequence at least 90% identical to SEQ ID NO: 4.

In some embodiments, the nucleic acid of (a) encodes an amino acid sequence at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% identical to SEQ ID NO: 2.

In some embodiments, the nucleic acid of (b) encodes an amino acid sequence at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% identical to SEQ ID NO: 4.

In some embodiments, the nucleic acid of (a) comprises SEQ ID NO: 1, or a sequence at least 75% identical thereto. In some embodiments, the nucleic acid of (a) comprises SEQ ID NO: 1, or a sequence at least 85% identical thereto. In some embodiments, the nucleic acid of (a) comprises a sequence at least at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 1.

In some embodiments, the nucleic acid of (a) comprises SEQ ID NO: 28, or a sequence at least 75% identical thereto. In some embodiments, the nucleic acid of (a) comprises SEQ ID NO: 28, or a sequence at least 85% identical thereto. In some embodiments, the nucleic acid of (a) comprises a sequence at least at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 28.

In some embodiments, the nucleic acid of (b) comprises SEQ ID NO: 3, or a sequence at least 75% identical thereto. In some embodiments, the nucleic acid of (b) comprises SEQ ID NO: 3, or a sequence at least 85% identical thereto. In some embodiments, the nucleic acid of (b) comprises a sequence at least at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 3.

In some embodiments, the nucleic acid of (b) comprises SEQ ID NO: 29, or a sequence at least 75% identical thereto. In some embodiments, the nucleic acid of (b) comprises SEQ ID NO: 29, or a sequence at least 85% identical thereto. In some embodiments, the nucleic acid of (b) comprises a sequence at least at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 29.

In some embodiments, the present disclosure provides a recombinant nucleic acid sequence that encodes an amino acid sequence that shares at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 2, and plants comprising said sequences.

In some embodiments, the present disclosure provides a recombinant nucleic acid sequence that encodes an amino acid sequence that shares at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 4, and plants comprising said sequences.

In some embodiments, the present disclosure provides a recombinant nucleic acid sequence that shares at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 1, and plants comprising said sequences.

In some embodiments, the present disclosure provides a recombinant nucleic acid sequence that shares at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 28, and plants comprising said sequences.

In some embodiments, the present disclosure provides a recombinant nucleic acid sequence that shares at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 3, and plants comprising said sequences.

In some embodiments, the present disclosure provides a recombinant nucleic acid sequence that shares at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 29, and plants comprising said sequences.

In some embodiments, the disclosure relates to a plant transformation vector comprising at least one of the sequences disclosed herein. In some embodiments, the disclosure relates to a plant transformation vector comprising at least one of: a nucleotide sequence encoding an amino acid sequence at least 90% identical to SEQ ID NO: 2; a nucleotide sequence encoding an amino acid sequence at least 90% identical to SEQ ID NO: 4; a nucleotide sequence comprising SEQ ID NO: 1; a nucleotide sequence at least 75% identical to SEQ ID NO: 1; a nucleotide sequence comprising SEQ ID NO: 28; a nucleotide sequence at least 75% identical to SEQ ID NO: 28; a nucleotide sequence comprising SEQ ID NO: 3; a nucleotide sequence at least 75% identical to SEQ ID NO: 3; a nucleotide sequence comprising SEQ ID NO: 29; and, a nucleotide sequence at least 75% identical to SEQ ID NO: 29.

Methods of alignment of sequences for comparison are well known in the art. Various programs and alignment algorithms are described in: Smith and Waterman (Adv. Appl. Math., 2:482, 1981); Needleman and Wunsch (J. Mol. Biol., 48:443, 1970); Pearson and Lipman (Proc. Natl. Acad. Sci., 85:2444, 1988); Higgins and Sharp (Gene, 73:237 44, 1988); Higgins and Sharp (CABIOS, 5:151 53, 1989); Corpet et al. (Nuc. Acids Res., 16:10881 90, 1988); Huang et al. (Comp. Appls Biosci., 8:155 65, 1992); and Pearson et al. (Meth. Mol. Biol., 24:307 31, 1994). Altschul et al. (Nature Genet., 6:119 29, 1994) presents a detailed consideration of sequence alignment methods and homology calculations.

In some embodiments, the disclosure relates to a plant transformation vector designated pSIM6102 comprising the FUS1 allele driven by its native promoter and terminator.

In some embodiments, the disclosure relates to a plant transformation vector designated pSIM6103 comprising the FUS1 allele excluding untranslated regions driven by the F. ananassa M8 constitutive promoter.

In some embodiments, the disclosure relates to a plant transformation vector designated pSIM6145 comprising the FUS1 and FUS4 alleles driven by their native promoters and terminators.

In some embodiments, the disclosure relates to a plant transformation vector designated pSIM6274 comprising the FUS4 allele driven by its native promoter and terminator.

In some embodiments, expression of the nucleotide sequence in the plant prevents Fusarium from colonizing the plant, or prevents Fusarium from affecting plant growth or yield. In some embodiments, the Fusarium spp. is Fusarium oxysporum.

In some embodiments, the disclosure relates to plants and plant parts transformed with the sequences and vectors disclosed herein. In some embodiments, the plant is resistant to a fungal pathogen.

The disclosure further relates to use of such plants to transfer resistance to other strawberry lines via a plant breeding technique. Example plant breeding techniques include, but are not limited to, recurrent selection, mass selection, hybridization, open-pollination, backcrossing, pedigree breeding, mutation breeding, and marker enhanced selection.

Naing et al. (2019, Plant Methods, 15:36, 10 pages) provide in vitro propagation methods for the production of morphologically and genetically stable plants of different strawberry cultivars using meristem cultures.

### Expression Vectors for Transformation: Marker Genes

Expression vectors usually include at least one genetic marker, operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

Some commonly used selectable marker genes for plant transformation include, but are not limited to, neomycin phosphotransferase II (nptII) (Fraley et al., Proc. Natl. Acad. Sci. U.S.A., 80:4803 (1983)), aminoglycoside phosphotransferases APH(3′)II and APH(3′)I (Davies and Smith, 1978; Jimenez and Davies, 1980), kanamycin resistance (KmR) gene(Gray and Fitch, 1983), hygromycin phosphotransferase gene (Vanden Elzen et al., Plant Mol. Biol., 5:299 (1985)), gentamycin acetyl transferase, streptomycin phosphotransferase, aminoglycoside-3′-adenyl transferase, and the bleomycin resistance determinant. Hayford et al., Plant Physiol. 86:1216 (1988), Jones et al., Mol. Gen. Genet., 210:86 (1987), Svab et al., Plant Mol. Biol. 14:197 (1990) Hille et al., Plant Mol. Biol. 7:171 (1986). Other selectable marker genes confer resistance to herbicides such as glyphosate, glufosinate or bromoxynil. Comai et al., Nature 317:741-744 (1985), Gordon-Kamm et al., Plant Cell 2:603-618 (1990) and Stalker et al., Science 242:419-423 (1988).

Selectable marker genes for plant transformation not of bacterial origin include, for example, mouse dihydrofolate reductase, plant 5-enolpyruvylshikimate-3-phosphate synthase and plant acetolactate synthase. Eichholtz et al., Somatic Cell Mol. Genet. 13:67 (1987), Shah et al., Science 233:478 (1986), Charest et al., Plant Cell Rep. 8:643 (1990).

Another class of marker genes for plant transformation requires screening of presumptively transformed plant cells rather than direct genetic selection of transformed cells for resistance to a toxic substance such as an antibiotic. Commonly used genes for screening presumptively transformed cells include beta-glucuronidase (GUS), beta-galactosidase, luciferase and chloramphenicol acetyltransferase. Jefferson, R. A., Plant Mol. Biol. Rep. 5:387 (1987), Teeri et al., EMBO J. 8:343 (1989), Koncz et al., Proc. Natl. Acad. Sci. USA 84:131 (1987), DeBlock et al., EMBO J. 3:1681 (1984). In vivo methods for visualizing GUS activity that do not require destruction of plant tissue are available. Molecular Probes publication 2908, IMAGENE GREEN, p. 1-4 (1993) and Naleway et al., J. Cell Biol. 115:151a (1991). Expression Vectors for

### Transformation: Promoters

Genes included in expression vectors are typically driven by a nucleotide sequence comprising a regulatory element, for example, a promoter. Several types of promoters are well known in the transformation arts as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters that initiate transcription only in a certain tissue are referred to as “tissue-specific”. A “cell-type” specific promoter primarily drives expression in certain cell types in one or more organs, for example, vascular cells in roots or leaves. See for example, Schaart J. G. et al. Isolation and characterization of a strawberry fruit-specific promoter. Genes, Genomes and Genomics 5 (Special Issue 1), 102-107 (2011). An “inducible” promoter is a promoter which is under environmental control.

Examples of environmental conditions that may affect transcription by inducible promoters include anaerobic conditions or the presence of light. Tissue-specific, tissue-preferred, cell type specific, and inducible promoters constitute the class of “non-constitutive” promoters. A “constitutive” promoter is a promoter that is active under most environmental conditions, and cell types.

### A. Inducible Promoters

An inducible promoter is operably linked to a gene for expression in a plant. Optionally, the inducible promoter is operably linked to a nucleotide sequence encoding a signal sequence which is operably linked to a gene for expression in a plant. With an inducible promoter the rate of transcription increases in response to an inducing agent.

Any inducible promoter can be used in the instant disclosure. See Ward et al., Plant Mol. Biol. 22:361-366 (1993). Exemplary inducible promoters include, but are not limited to, that from the ACEI system which responds to copper (Mett et al., PNAS 90:4567-4571 (1993)); In2 gene from maize which responds to benzenesulfonamide herbicide safeners (Hershey et al., Mol. Gen Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991)). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone. Schena et al., Proc. Natl. Acad. Sci. USA 88:0421 (1991).

### B. Constitutive Promoters

A constitutive promoter is operably linked to a gene for expression in a plant or the constitutive promoter is operably linked to a nucleotide sequence encoding a signal sequence which is operably linked to a gene for expression in a plant.

Many different constitutive promoters can be utilized in the instant disclosure. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2: 163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)).

The ALS promoter, Xbal/Ncol fragment 5′ to the Brassica napus ALS3 structural gene (or a nucleotide sequence similarity to said Xbal/Ncol fragment), represents a particularly useful constitutive promoter. See PCT application WO 96/30530.

### C. Tissue-Specific or Tissue-Preferred Promoters

A tissue-specific promoter is operably linked to a gene for expression in a plant. Optionally, the tissue-specific promoter is operably linked to a nucleotide sequence encoding a signal sequence which is operably linked to a gene for expression in a plant. Plants transformed with a gene of interest operably linked to a tissue-specific promoter produce the protein product of the transgene exclusively, or preferentially, in a specific tissue.

Any tissue-specific or tissue-preferred promoter can be utilized in the instant disclosure. Exemplary tissue-specific or tissue-preferred promoters include, but are not limited to, a root-preferred promoter—such as that from the phaseolin gene (Murai et al., Science 23:476-482 (1983) and Sengupta-Gopalan et al., Proc. Natl. Acad. Sci. USA 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993)).

### Expression Vectors for Transformation: Terminators

As used herein, the term “terminator” or “termination sequence” generally refers to a 3′ flanking region of a gene that contains nucleotide sequences which regulate transcription termination and typically confer RNA stability.

The disclosure provides terminator sequences that find use in proper transcriptional processing of recombinant nucleic acids in the transformation vectors taught herein. Although terminator sequences do not by themselves initiate gene transcription, their presence can increase accurate processing and termination of the RNA transcript, and result in message stability. The use of recombinant terminator sequences is established in the art. It is appreciated that an understanding of the molecular mechanisms underlying terminator sequence activity are not required to make or use the present disclosure.

### Methods for Transformation

Transformation methods and vectors for strawberry are well known in the art. See for example, Mathews, H., et al. Genetic transformation of strawberry: Stable integration of a gene to control biosynthesis of ethylene. In Vitro Cell Dev Biol—Plant 31, 36-43 (1995); Nehra N S, et al. Genetic transformation of strawberry by Agrobacterium tumefaciens using a leaf disk regeneration system. Plant Cell Rep. 1990 October; 9(6):293-8; Schaart, J. G. (2014). Agrobacterium-mediated Transformation of Strawberry. Bio-protocol 4(1); Ricardo V. G. et al., Transformation of a Strawberry Cultivar Using a Modified Regeneration Medium. HortScience 38(2):277-280 (2003); Yang, X., et al. Strawberry vein banding virus-based vector for transient overexpression in strawberry plants. Phytopathol Res 4, 8 (2022); Quesada M. A. et al., Transgenic Strawberry: Current Status and Future Perspectives. Transgenic Plant Journal 1(2):280-288 (2007).

Additional methods for plant transformation have been developed and are well known in the art, including biological and physical plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc. Boca Raton, 1993) pages 67-88. In addition, expression vectors and in-vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pages 89-119.

### A. Agrobacterium-Mediated Transformation

One method for introducing an expression vector into plants is based on the natural transformation system of Agrobacterium. U.S. Pat. No. 6,274,791 provides methods for agrobacterium-mediated transformation and regeneration of strawberry plants using explants prepared from leaves. U.S. Pat. No. 6,043,410 isolated promoters from genomic DNA of strawberry plants and showed that the promoters are capable of tissue-specific expression in transgenic strawberry plants produced via regeneration of apical meristem tissues.

There are numerous patents governing Agrobacterium mediated transformation and particular DNA delivery plasmids designed specifically for use with Agrobacterium, for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. Nos. 5,693,512, 6,051,757 and EP904362A1, which are all hereby incorporated by reference in their entirety.

Although transgenic plants produced through Agrobacterium-mediated transformation generally contain a simple integration pattern as compared to microparticle-mediated genetic transformation, a wide variation in copy number and insertion patterns exists (Jones et al, 1987; Jorgensen et al., 1987). Moreover, even within a single plant genotype, different patterns of transfer DNA integration are possible based on the type of explant and transformation system used (Grevelding et al., 1993).

### B. Direct Gene Transfer

Direct plant transformation methods using DNA have also been reported. The first of these to be reported historically is electroporation, which utilizes an electrical current applied to a solution containing plant cells (M. E. Fromm et al., Nature, 319, 791 (1986); H. Jones et al., Plant Mol. Biol., 13, 501 (1989) and H. Yang et al., Plant Cell Reports, 7, 421 (1988).

Another direct method, called “biolistic bombardment”, uses ultrafine particles, usually tungsten or gold, that are coated with DNA and then sprayed onto the surface of a plant tissue with sufficient force to cause the particles to penetrate plant cells, including the thick cell wall, membrane and nuclear envelope, but without killing at least some of them (U.S. Pat. Nos. 5,204,253, 5,015,580).

A third direct method uses fibrous forms of metal or ceramic consisting of sharp, porous or hollow needle-like projections that literally impale the cells, and also the nuclear envelope of cells. Both silicon carbide and aluminum borate whiskers have been used for plant transformation (Mizuno et al., 2004; Petolino et al., 2000; U.S. Pat. No. 5,302,523 US Application 20040197909) and also for bacterial and animal transformation (Kaepler et al., 1992; Raloff, 1990; Wang, 1995). There are other methods reported, and undoubtedly, additional methods will be developed. The methods taught herein are capable of detecting the non-naturally occurring nucleotide junctions that result from any plant transformation method.

The sequences of the present disclosure may be transferred to any cell, for example, such as a plant cell transformation competent bacterium. Such bacteria are known in the art and may, for instance, belong to the following species: Agrobacterium spp., Rhizobium spp., Sinorhizobium spp., Mesorhizobium spp., Phyllobacterium spp. Ochrobactrum spp. and Bradyrhizobium spp. In some embodiments, such bacteria may belong to Agrobacterium spp.

The present disclosure also relates to a plant cell transforming bacterium comprising the sequences disclosed herein, and which may be used for transforming a plant cell. In some embodiments, the plant transforming bacteria is selected from Agrobacterium spp., Rhizobium spp., Sinorhizobium spp., Mesorhizobium spp., Phyllobacterium spp. Ochrobactrum spp. or Bradyrhizobium spp.

The present disclosure also relates to a method for transforming a plant cell comprising: contacting at least a first plant cell with a plant cell transforming bacteria of the present disclosure; and selecting at least a plant cell transformed with one or more of the sequences disclosed herein. In some embodiments, the plant cell is a strawberry plant cell. In one embodiment, a method of the disclosure further comprises regenerating a plant from the plant cell.

In some embodiments, the present disclosure provides a method for transforming a plant cell, wherein the method comprises: (i) introducing a plant transformation vector taught herein into the plant cell; and (ii) cultivating the transformed plant cell under conditions conducive to regeneration and mature plant growth. In some embodiments, the plant cell is a strawberry cell.

Persons of ordinary skill in the art will recognize that plants comprising the sequences disclosed herein also includes derivative varieties that retain the essential distinguishing characteristics of the event in question, such as a locus converted plant of that variety or a derivative having one or more value-added genes incorporated therein (such as herbicide or pest resistance).

Likewise, transgenes can be introduced into the plant using any of a variety of established recombinant methods well-known to persons skilled in the art, such as: Gressel, 1985, Biotechnologically Conferring Herbicide Resistance in Crops: The Present Realities, In Molecular Form and Function of the Plant Genome, L. van Vloten-Doting, (ed.), Plenum Press, New York; Huttner, S. L., et al., 1992, Revising Oversight of Genetically Modified Plants, Bio/Technology; Klee, H., et al., 1989, Plant Gene Vectors and Genetic Transformation: Plant Transformation Systems Based on the use of Agrobacterium tumefaciens, Cell Culture and Somatic Cell Genetics of Plants; Koncz, C., et al., 1986, The Promoter of T. sub.L-DNA Gene 5 Controls the Tissue-Specific Expression of Chimeric Genes Carried by a Novel Type of Agrobacterium Binary Vector; Molecular and General Genetics; Lawson, C., et al., 1990; all of which are incorporated herein by reference for this purpose.

Methods of modifying nucleic acid constructs to increase expression levels in plants are also generally known in the art (see, e.g. Rogers et al., 260 J. Biol. Chem. 3731-38, 1985; Cornejo et al., 23 Plant Mol. Biol. 567: 81, 1993). In engineering a plant system to affect the rate of transcription of a protein, various factors known in the art, including regulatory sequences such as positively or negatively acting sequences, enhancers and silencers, as well as chromatin structure may have an impact. The present disclosure provides that at least one of these factors may be utilized in engineering plants to express a protein of interest. The sequences of the present disclosure are native genetic elements, i.e., are isolated from the selected plant species to be modified.

### Engineering Fusarium Wilt Resistance Via Targeted Genome Editing

Using genome editing, DNA can be modified in a targeted way providing new alternatives to develop traits in plants.

Genome editing by CRISPR, which stands for Clustered Regularly Interspaced Short Palindromic Repeats, is based on a natural immune process used by bacteria to defend themselves against invading viruses. Indeed, in bacteria the invading viral DNA will be cut through use of a guide RNA (gRNA), or piece of RNA, and a CRISPR-associated protein (Cas). The last step of the bacterial immune process, when the gRNA is combined with Cas and cleaves the target DNA, has been adopted for genome editing in laboratories.

There are at least three main CRISPR system types (Type I, II, and III) and at least 10 distinct subtypes (Makarova, K. S., et. al., Nat Rev Microbiol. 2011 May 9; 9(6):467-477). Type I and III systems use Cas protein complexes and short guide polynucleotide sequences to target selected DNA regions. Type II systems rely on a single protein (e.g. Cas9) and the targeting guide polynucleotide, where a portion of the 5′ end of a guide sequence is complementary to a target nucleic acid. For more information on the CRISPR gene editing compositions and methods of the present disclosure, see U.S. Pat. Nos. 8,697,359; 8,889,418; 8,771,945; and 8,871,445, each of which is hereby incorporated in its entirety for all purposes.

CRISPR genome editing requires two components, a gRNA and a Cas enzyme. These components associate to form a ribonucleoprotein (RNP) complex, where after the gRNA can base pair with a complementary protospacer sequence (i.e. the target genomic sequence of about 20 bases in length) under the condition that a particular adjacent sequence, called a protospacer-adjacent motif (PAM), is present in the genome. The PAM is only a few bases long, and its sequence depends on the type of Cas enzyme used. Once the gRNA binds to the target DNA (protospacer), the Cas enzyme recognizes this complex and makes a precise cut at the target site, resulting in a double strand break (DSB).

Each Cas enzyme is directed by the gRNA to a user-specified cut site in the genome. Like Cas9 nucleases, Cas12a1 family members contain a RuvC-like endonuclease domain, but lack the second HNH endonuclease domain of Cas9. Cas12a cleaves DNA in a staggered pattern in contrast to Cas9 which produces a blunt-end. Moreover, for cleavage Cas12a requires only one RNA rather than the two tracrRNA and crRNA needed by Cas9. For Cas9 as well as Cas12a, the target sequence of the gRNAs must be next to a PAM sequence. In the case of Cas9, the PAM sequence corresponds to NGG, where N is any base. The gRNA will recognize and bind to 20 nucleotides on the DNA strand opposite from the NGG PAM site. For Cas12a, the PAM sequence is TTTV, where V can represent A, C, or G. Using Alt-R Cas12a Ultra from Integrated DNA Technologies, a TTTT PAM sequence may also work. The “V” of the TTTV is immediately adjacent to the base at the 5′ end of the non-targeted strand side of the protospacer element. The guide RNA for Cas12a is relatively short and is approximately 40 to 44 bases long.

The damage caused by the DSB will be repaired in eukaryotic cells, primarily by two pathways: Non-Homologous End-Joining (NHEJ) and Homology Directed Repair (HDR). The HDR mechanism requires the presence of a donor DNA template containing regions of homology to both sites of the DNA break. This donor DNA can carry specific mutations and has to be delivered simultaneously with a preassembled Cas RNP complex composed of Cas9 or Cas12a and synthetically produced gRNAs.

Altogether, targeted cleavage events induced by nucleases can be used to introduce targeted mutations (deletions, substitutions and insertions) in genomic DNA sequences and as such, can be used as an efficient tool for genome editing in plants.

In some aspects, the present disclosure relates to a recombinant DNA construct comprising an expression cassette(s) encoding a site-specific nuclease and, optionally, any associated protein(s) to carry out genome modification. These nuclease-expressing cassette(s) may be present in the same molecule or vector as a donor template for templated editing or an expression cassette comprising nucleic acid sequence encoding a FUS1 and/or FUS4 allele as described herein or on a separate molecule or vector. Several methods for site-directed integration are known in the art involving different sequence-specific nucleases (or complexes of proteins or guide RNA or both) that cut the genomic DNA to produce a DSB or nick at a desired genomic site or locus. As understood in the art, during the process of repairing the DSB or nick introduced by the nuclease enzyme, the donor template DNA, transgene, or expression cassette may become integrated into the genome at the site of the DSB or nick. The presence of the homology arm(s) in the DNA to be integrated may promote the adoption and targeting of the insertion sequence into the plant genome during the repair process through homologous recombination, although an insertion event may occur through non-homologous end joining (NHEJ).

In some aspects, the endonuclease is selected from a meganuclease, a zinc-finger nuclease (ZFN), a transcription activator-like effector nucleases (TALEN), an Argonaute (non limiting examples of Argonaute proteins include Thermus thermophilus Argonaute (TtAgo), Pyrococcus furiosus Argonaute (PfAgo), Natronobacterium gregoryi Argonaute (NgAgo), an RNA-guided nuclease, such as a CRISPR associated nuclease (non-limiting examples of CRISPR associated nucleases include Cas1, Cas1B, Cas2, Cas3, Cas4, Cas5, Cas6, Cas7, Cas8, Cas9 (also known as Csn1 and Csx12), Cas12a (also known as Cpf1), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csxl, Csx15, Csf1, Csf2, Csf3, Csf4, Cpf1, CasX, CasY, homologs thereof, or modified versions thereof.

In some aspects, the site-specific genome modification enzyme is a recombinase. Non-limiting examples of recombinases include a tyrosine recombinase attached to a DNA recognition motif provided herein is selected from the group consisting of a Cre recombinase, a Gin recombinase, a Flp recombinase, and a Tnpl recombinase. In some aspects, a Cre recombinase or a Gin recombinase provided herein is tethered to a zinc-finger DNA-binding domain, or a TALE DNA-binding domain, or a Cas9 nuclease. In another aspect, a serine recombinase attached to a DNA recognition motif provided herein is selected from the group consisting of a PhiC3 1 integrase, an R4 integrase, and a TP-901 integrase. In another aspect, a DNA transposase attached to a DNA binding domain provided herein is selected from the group consisting of a TALE-piggyBac and TALE-Mutator.

In some aspects, plants comprising one or more of the genetic alterations described herein may be selfed or crossed to produce lines that are homozygous for one or more of the genetic alterations described herein. In some aspects, the genetic alterations described herein may be transferred or introgressed to other strawberry varieties through conventional breeding schemes.

In some embodiments, the genetic alteration comprises replacing isoleucine with phenylalanine at a position corresponding to 256 of SEQ ID NO: 4; replacing serine with tryptophan at a position corresponding to 444 of SEQ ID NO: 4; replacing serine with glycine at a position corresponding to 509 of SEQ ID NO: 4; and/or replacing alanine with threonine at a position corresponding to 545 of SEQ ID NO: 4.

In some embodiments, the genetic alteration comprises a deletion of two prolines at a position corresponding to 1,122-1,123 of SEQ ID NO: 7.

In some embodiments, the genetic alteration comprises replacing isoleucine with serine at a position corresponding to 1,046 of SEQ ID NO: 2; replacing leucine with phenylalanine at a position corresponding to 1,060 of SEQ ID NO: 2; replacing cysteine with serine at a position corresponding to 1,063 of SEQ ID NO: 2; replacing phenylalanine with leucine at a position corresponding to 1,065 of SEQ ID NO: 2; replacing isoleucine with asparagine at a position corresponding to 1,069 of SEQ ID NO: 2; replacing asparagine with tyrosine at a position corresponding to 1,072 of SEQ ID NO: 2; replacing glutamate with lysine at a position corresponding to 1,073 of SEQ ID NO: 2; replacing cysteine with phenylalanine at a position corresponding to 1,083 of SEQ ID NO: 2; replacing phenylalanine with valine at a position corresponding to 1,121 of SEQ ID NO: 2; replacing tyrosine with histidine at a position corresponding to 1,122 of SEQ ID NO: 2; replacing tyrosine with cysteine at a position corresponding to 1,123 of SEQ ID NO: 2; replacing arginine with proline at a position corresponding to 1,126 of SEQ ID NO: 2; and/or replacing asparagine with arginine at a position corresponding to 1,130 of SEQ ID NO: 2.

In some embodiments, genetic alteration comprises replacing glycine with alanine at a position corresponding to 1,078 of SEQ ID NO:2; replacing leucine with isoleucine at a position corresponding to 1,079 of SEQ ID NO:2; replacing serine with threonine at a position corresponding to 1,120 of SEQ ID NO: 2; and/or replacing valine with leucine at a position corresponding to 1,153 of SEQ ID NO: 2.

See also Zhou J. et al., Efficient genome editing of wild strawberry genes, vector development and validation. Plant Biotechnology J (2018) 16:11 pp 1868-1877.

Plants Produced from the Methods Disclosed Herein

In some embodiments, the disclosure relates to cisgenic Fragaria spp. plants produced from the disclosed methods. In some embodiments, the disclosure relates to transgenic Fragaria spp. plants produced from the disclosed methods. In some embodiments, the disclosure relates to genetically engineered Fragaria spp. plants produced from the disclosed methods. In some embodiments, the disclosure relates to Fragaria spp. plants produced from the disclosed marker-assisted breeding methods.

In some embodiments, the transgenic Fragaria spp. plant, wherein the nucleic acid encoding an amino acid sequence at least 90% identical to SEQ ID NO: 2, and/or the nucleic acid encoding an amino acid sequence at least 90% identical to SEQ ID NO: 4 is operably linked to a constitutive promoter.

In some embodiments, the transgenic Fragaria spp. plant has 2 copies, 3 copies, 4 copies, 5 copies, 6 copies, 7 copies, 8 copies, or more than 9 copies of the nucleic acid encoding an amino acid sequence at least 90% identical to SEQ ID NO: 2, and/or the nucleic acid encoding an amino acid sequence at least 90% identical to SEQ ID NO: 4.

In some embodiments, the transgenic Fragaria spp. plant has between 2 and 4, between 4 and 6, between 6 and 8, or 9 or more copies of the nucleic acid encoding an amino acid sequence at least 90% identical to SEQ ID NO: 2, and/or the nucleic acid encoding an amino acid sequence at least 90% identical to SEQ ID NO: 4.

In some embodiments, the cisgenic or transgenic Fragaria spp. plant has between 1.1-3 fold increase in expression of the nucleic acid encoding an amino acid sequence at least 90% identical to SEQ ID NO: 2 compared to a non-transgenic Fragaria spp. plant.

In some embodiments, the cisgenic or transgenic Fragaria spp. plant has between 1.1-3 fold increase in expression of the nucleic acid encoding an amino acid sequence at least 90% identical to SEQ ID NO: 4 compared to a non-transgenic Fragaria spp. plant.

In some embodiments, the cisgenic or transgenic Fragaria spp. plant has between a 3- and 5-fold increase in expression of the nucleic acid encoding an amino acid sequence at least 90% identical to SEQ ID NO: 4 and/or the nucleic acid encoding an amino acid sequence at least 90% identical to SEQ ID NO: 2 compared to a non-transgenic Fragaria spp. plant.

In some embodiments, the cisgenic or transgenic Fragaria spp. plant has between a 5- and 10-fold increase in expression of the nucleic acid encoding an amino acid sequence at least 90% identical to SEQ ID NO: 4 and/or the nucleic acid encoding an amino acid sequence at least 90% identical to SEQ ID NO: 2 compared to a non-transgenic Fragaria spp. plant.

In some embodiments, the cisgenic or transgenic Fragaria spp. plant has between a 10- and 15-fold increase in expression of the nucleic acid encoding an amino acid sequence at least 90% identical to SEQ ID NO: 4 and/or the nucleic acid encoding an amino acid sequence at least 90% identical to SEQ ID NO: 2 compared to a non-transgenic Fragaria spp. plant.

In some embodiments, the cisgenic or transgenic Fragaria spp. plant has between a 15- and 20-fold increase in expression of the nucleic acid encoding an amino acid sequence at least 90% identical to SEQ ID NO: 4 and/or the nucleic acid encoding an amino acid sequence at least 90% identical to SEQ ID NO: 2 compared to a non-transgenic Fragaria spp. plant.

In some embodiments, the cisgenic or transgenic Fragaria spp. plant has a 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0-fold increase in expression of the nucleic acid encoding an amino acid sequence at least 90% identical to SEQ ID NO: 4 and/or the nucleic acid encoding an amino acid sequence at least 90% identical to SEQ ID NO: 2 compared to a non-transgenic Fragaria spp. plant.

In some embodiments, the Fragaria spp. plant, cisgenic Fragaria spp. plant, or transgenic Fragaria spp. plant described herein has a Fusarium wilt resistance rAUDPC score of less than 0.04, less than 0.03, less than 0.02, or less than 0.01.

In some embodiments, the Fragaria spp. plant, cisgenic Fragaria spp. plant, or transgenic Fragaria spp. plant described herein has a Fusarium wilt resistance rAUDPC score of between 0.01 and 0.02, between 0.02 and 0.03, or between 0.03 and 0.04.

In some embodiments, the disclosure relates to a genetically engineered Fragaria spp. plant or plant part including a Fusarium wilt resistance allele designated FUS4, wherein said FUS4 allele encodes a protein including SEQ ID NO: 4, or a sequence at least 90% identical thereto, wherein the protein encoded by said allele is a leucine rich receptor like kinase, and wherein said genetically engineered Fragaria spp. plant or plant part has a Fusarium wilt resistance rAUDPC score of less than 0.04. In some embodiments, the genetically engineered Fragaria spp. plant or plant part has an average Fusarium wilt resistance rAUDPC score of less than 0.03, less than 0.02, less than 0.01.

In some embodiments, the FUS4 allele encodes a protein that is at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% identical to SEQ ID NO: 4.

In some embodiments, the genetically engineered Fragaria spp. plant or plant part has an FUS4 allele comprising SEQ ID NO: 3, or a sequence at least 75% identical thereto, or SEQ ID NO: 29, or a sequence at least 75% identical thereto.

In some embodiments, the disclosure relates to a genetically engineered Fragaria spp. plant or plant part including a Fusarium wilt resistance allele designated FUS1, wherein said FUS1 allele encodes a protein including SEQ ID NO: 2, or a sequence at least 90% identical thereto, wherein said FUS1 allele encodes a TIR-NB-ARC-LRR protein that includes in its amino acid sequence a C-terminal Jelly-roll/Ig-like Domain, and wherein said genetically engineered Fragaria spp. plant or plant part has an average Fusarium wilt resistance rAUDPC score of less than 0.04. In some embodiments, the genetically engineered Fragaria spp. plant or plant part has an average Fusarium wilt resistance rAUDPC score of less than 0.03, less than 0.02, less than 0.01.

In some embodiments, the FUS1 allele encodes a protein that is at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% identical to SEQ ID NO: 2.

In some embodiments, the genetically engineered Fragaria spp. plant or plant part has an FUS1 allele comprising SEQ ID NO: 1, or a sequence at least 75% identical thereto, or SEQ ID NO: 28, or a sequence at least 75% identical thereto.

In some embodiments, the genetically engineered Fragaria spp. plant or plant part having an FUS1 and/or FUS4 allele has a Fusarium wilt resistance rAUDPC score of between 0.01 and 0.02, between 0.02 and 0.03, or between 0.03 and 0.04.

In some aspects, the techniques described herein relate to a genetically engineered Fragaria spp. plant or plant part, wherein the FUS4 allele is driven by its native promoter.

In some aspects, the techniques described herein relate to a genetically engineered Fragaria spp. plant or plant part, wherein the FUS4 allele is driven by a constitutive promoter.

In some aspects, the techniques described herein relate to a genetically engineered Fragaria spp. plant or plant part, wherein the plant or plant part has increased FUS4 expression compared to a non-transgenic Fragaria spp. plant or plant part.

In some aspects, the techniques described herein relate to a genetically engineered Fragaria spp. plant or plant part, wherein the expression of FUS4 is between 1.1 and 20-fold greater than a non-transgenic Fragaria spp. plant or plant part.

In some aspects, the techniques described herein relate to a genetically engineered Fragaria spp. plant or plant part, wherein the expression of FUS4 is between 1.1 and 10-fold greater than a non-transgenic Fragaria spp. plant or plant part.

In some aspects, the techniques described herein relate to a genetically engineered Fragaria spp. plant or plant part, wherein the expression of FUS4 is between 10 and 20-fold greater than a non-transgenic Fragaria spp. plant or plant part.

In some aspects, the techniques described herein relate to a genetically engineered Fragaria spp. plant or plant part, wherein the FUS1 allele is driven by its native promoter.

In some aspects, the techniques described herein relate to a genetically engineered Fragaria spp. plant or plant part, wherein the FUS1 allele is driven by a constitutive promoter.

In some aspects, the techniques described herein relate to a genetically engineered Fragaria spp. plant or plant part, wherein the plant or plant part has increased FUS1 expression compared to a non-transgenic Fragaria spp. plant or plant part.

In some aspects, the techniques described herein relate to a genetically engineered Fragaria spp. plant or plant part, wherein the expression of FUS1 is between 1.1 and 20-fold greater than a non-transgenic Fragaria spp. plant or plant part.

In some aspects, the techniques described herein relate to a genetically engineered Fragaria spp. plant or plant part, wherein the expression of FUS1 is between 1.1 and 10-fold greater than a non-transgenic Fragaria spp. plant or plant part.

In some aspects, the techniques described herein relate to a genetically engineered Fragaria spp. plant or plant part, wherein the expression of FUS1 is between 10 and 20-fold greater than a non-transgenic Fragaria spp. plant or plant part.

### Resistance to Other Pests and Pathogens and Other Desirable Traits

As will be understood by one skilled in the art, additional alleles may be stacked in the DNA constructs disclosed herein, or plants may be further bred to confer other desirable traits, and/or tolerance or resistance to other pests and pathogens. Methods for stacking genes are well known in the art. For example, the GAANTRY system (Gene Assembly in Agrobacterium by Nucleic acid Transfer using Recombinase technology) leverages recombinase-mediated stacking technology. The specificity and efficiency of recombinases make them extremely attractive for genome engineering. Advancements in molecular biology and recombinases have paved the way for gene stacking with the assistance of unidirectional recombination systems. Development of this high-efficiency gene stacking system uses the specificity of the recombinases to effectively deliver the target genes of interest to a predetermined position. This is a flexible and effective system for stably stacking multiple genes within an Agrobacterium virulence plasmid Transfer-DNA (T-DNA).

Examples of agronomically important traits include but are not limited to those that result in increased biomass production, increased food production, improved food quality, increased fruit production. Additional examples of agronomically important traits includes pest resistance, vigor, development time (time to harvest), enhanced nutrient content, novel growth patterns, flavors or colors, salt, heat, drought and cold tolerance, disease resistance, fruit size, fruit weight, fruit color, fruit nutrients, fruit taste, and the like.

Non-limited examples of disease resistance include, resistant to Raspberry ringspot virus (RpRSV), Strawberry crinkle virus (SCV), Strawberry feather leaf virus, Strawberry latent “C” virus (SLCV), Strawberry latent ringspot virus (SLRSV), Strawberry leaf roll virus, Strawberry mild yellow edge virus (SMYEV), Strawberry mottle virus (SMV), Strawberry pallidosis virus, Strawberry vein banding virus (SVBV), Tobacco necrosis virus (TNV), Tobacco ringspot virus (TRSV), Tobacco streak virus (TSV), Strawberry necrotic shock virus (SNSV), Tomato black ring virus (TBRV), Tomato bushy stunt virus (TBSV), Tomato ringspot virus (ToRSV), and Xanthamonas Fragariae (angular leafspot). Additional preferred traits are described in Yue et al. (An Evaluation of U.S. Strawberry Producers Trait Prioritization: Evidence from Audience Surveys, HortScience 49(2) 188-193 (2014)).

### Plant Breeding Techniques

Marker-assisted breeding or marker enhanced selection for the FUS1 and FUS4 alleles can be used with any plant breeding technique. Additionally, plants comprising the FUS1 and/or FUS4 alleles from either transformation or genetic engineering may further be bred, and the trait transferred to other lines. In some embodiments, the plant breeding technique is selected from the group consisting of recurrent selection, mass selection, hybridization, open-pollination, backcrossing, pedigree breeding, mutation breeding, haploid/double haploid production, and marker enhanced selection for other desirable traits. In some embodiments, the plant breeding technique is mutation breeding and the mutation selected is spontaneous or artificially induced.

Traditional methods for breeding strawberry plants can be utilized to create additional strawberry plants based on the present disclosure, such as those described in Strawberry; History, Breeding and Physiology by Darrow GM (1966), and U.S. Pat. No. 6,598,339 which is hereby incorporated by reference in its entirety. The cultivated strawberry (F. x ananassa) is an interspecific hybrid between the wild octaploid species F. chiloensis L. and F. virginiana Duch., which was first introduced in the 1750 s (Darrow, 1966). Using recurrent mass selection, intraspecific and interspecific crosses have been utilized to make new cultivars. There are more than twenty Fragaria species possessing multiple ploidy that change in size, color, flavor, shape, degree of fertility, season of ripening, susceptibility to disease and constitution of plant (Biswas et al. (2009) Sci Hortic. 122:409-416). With intraspecific crosses of the cultivated strawberry variety (F. x ananassa), improved agronomic traits are introduced into new cultivars. Pedigree selection, crossing of the best genotypes, and further selection are used for breeding for new strawberry cultivars because the strawberry cultivars are heterozygous and sensitive to inbreeding. Strawberry cultivars are then vegetatively propagated through runners (or stolons) as clones (Hancock, 1999).

Also, a new strawberry cultivar can be developed through the induction of somaclonal variation from in vitro tissue culture and selection of suitable variants for further cultivation (Biswas et al., 2009). Somaclonal variation occurs by changes in chromosome number (polyploidy) or chromosome rearrangements by insertions, deletions, translocations, or mutation. The success of plant breeding by somaclonal variation depends on the selection of genetically stable somaclones.

Classic breeding methods can be included in the present disclosure to introduce one or more modified gene of the present disclosure into other plant varieties, or other close-related species that are compatible to be crossed with the plants of the present disclosure.

For example, backcrossing methods can be used with the present disclosure to improve or introduce a characteristic into the variety. The term “backcrossing” as used herein refers to the repeated crossing 1, 2, 3, 4, 5, 6, 7, 8, 9 or more times of a hybrid progeny back to the recurrent parents. The parental plant which contributes the gene(s) for the one or more desired characteristics is termed the nonrecurrent or donor parent. This terminology refers to the fact that the nonrecurrent parent is used one time in the backcross protocol and therefore does not recur. The parental plant to which the gene or genes from the nonrecurrent parent are transferred is known as the recurrent parent as it is used for several rounds in the backcrossing protocol. In a typical backcross protocol, the original variety of interest (recurrent parent) is crossed to a second variety (nonrecurrent parent) that carries the gene(s) of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the one or more genes transferred from the nonrecurrent parent.

The selection of a suitable recurrent parent is an important step for a successful backcrossing procedure. The goal of a backcross protocol is to alter or substitute one or more traits or characteristics in the original variety. To accomplish this, one or more genes of the recurrent variety are modified, substituted or supplemented with the desired gene(s) from the nonrecurrent parent, while retaining essentially all of the rest of the desired genes, and therefore the desired physiological and morphological constitution of the original variety. The choice of the particular nonrecurrent parent will depend on the purpose of the backcross. One of the major purposes is to add some commercially desirable, agronomically important trait to the plant. The exact backcrossing protocol will depend on the characteristic or trait being altered or added to determine an appropriate testing protocol. Although backcrossing methods are simplified when the characteristic being transferred is a dominant allele, a recessive allele may also be transferred. In this instance, it may be necessary to introduce a test of the progeny to determine if the desired characteristic has been successfully transferred.

The present disclosure provides strawberry cultivars having resistance to Fusarium oxysporum, wherein the strawberry cultivars genetically trace their resistance to Fusarium oxysporum to a strawberry cultivar described herein.

The present disclosure provides methods for producing hybrid strawberry plants, or parts thereof, comprising crossing two strawberry plants to produce such hybrid strawberry plants and growing the hybrid strawberry plants, wherein at least one of the two strawberry plants is a strawberry cultivar described herein. In some embodiments, the hybrid strawberry plants display resistance to Fusarium oxysporum f. sp. Fragariae and are selected using molecular markers described herein.

Plants for Use with the Disclosed Methods and Sequences

The genetics of strawberry plants are uniquely diverse in terms of ploidy. Strawberry plant species can be diploid, tetraploid, pentaploid, hexaploid, heptaploid, octaploid, or decaploid (which have 2, 4, 5, 6, 7, 8, or 10 sets of chromosomes, respectively). Some species of Fragaria have uncategorized ploidy.

Fragaria species include but are not limited to, 1) diploid: F. bucharica, F. chinensis, F. daltoniana, F. gracilis, F. hayatai, F. iinumae, F. nilgerrensis, F. nipponica, F. nubicola, F. pentaphylla, F. rubicola, F. vesca, F. viridis, F. vezoensis, and F. x bifera; 2) tetraploid: F. corymbosa, F. moupinensis, F. orientalis, and F. tibetica; 3) pentaploid: F. x bringhurstii; 4) hexaploid: F. moschata; 5) hexaploid: F. moschata; 6) heptaploid: F. x comarum; 7) octaploid: F. x ananassa, F. chiloensis, F. chiloensis subsp. chiloensis forma chiloensis, F. chiloensis subsp. chiloensis forma patagonica, F. chiloensis subsp. Lucida, F. chiloensis subsp. Pacifica, Fragaria chiloensis subsp. Sandwicensis, F. iturupensis, F. ovalis, and F. virginiana; 8) decaploid: C. frutescens, C. chinense, and C. pendulum. More Fragaria species are described in Liston et al. (Fragaria: A genus with deep historical roots and ripe for evolutionary and ecological insights, American Journal of Botany (2014) 101:1686-1699) and Kole (Wild Crop relatives: Genomic and Breeding Resources: Temperate Fruits, Chapter 2 Fragaria, 2011).

Example fusarium susceptible strawberry lines for use with the disclosed methods and sequences include, but are not limited to, ‘Inspire’, ‘Benicia’, ‘Grenada’, ‘UCD Royal Royce’, ‘Seascape’, ‘Camarosa’, ‘Albion’, ‘Florida Belle’, ‘Jurica’, ‘Puget Reliance’, ‘Lassen’, ‘Brighton’, ‘Selkirk’, ‘Vystavochnaya’, and ‘Radiance’.

### SEQUENCE LISTING

TABLE 7A
Sequence Listing

| Sequence name | SEQ ID NO: |
|---|---|
| FUS1_R_gene (including UTRs) | SEQ ID NO: 1 |
| FUS1_R_protein (BG4352_39087, BG123258_58000, | SEQ ID NO: 2 |
| PS9271_PR.4.1.4.0) | |
| FUS4_RLK_gene (including UTRs) | SEQ ID NO: 3 |
| FUS4_RLK_protein (BG4352_39082, BG123258_57969) | SEQ ID NO: 4 |
| C-JID domain of FUS1 | SEQ ID NO: 5 |
| Inspire_2B-1 gene | SEQ ID NO: 6 |
| Inspire_2B-1 protein | SEQ ID NO: 7 |
| Fxa2Dg200066 gene | SEQ ID NO: 8 |
| Fxa2Dg200066 protein | SEQ ID NO: 9 |
| BG4352_39087, BG123258_58000, PS9271_PR.4.1.4.0 gene | SEQ ID NO: 10 |
| BG4352_39087, BG123258_58000, PS9271_PR.4.1.4.0 protein | SEQ ID NO: 11 |
| Fxa2Bg200175 gene | SEQ ID NO: 12 |
| Fxa2Bg200175 protein | SEQ ID NO: 13 |
| Inspire_2B-2 gene | SEQ ID NO: 14 |
| Inspire_2B-2 protein | SEQ ID NO: 15 |
| BG4352_39082, BG123258_57969 gene | SEQ ID NO: 16 |
| BG4352_39082, BG123258_57969 protein | SEQ ID NO: 17 |
| FUS1_R_gene (without UTRs) | SEQ ID NO: 28 |
| FUS4_RLK_gene (without UTRs) | SEQ ID NO: 29 |

TABLE 7B
Sequence Listing

| Construct/gene | Primer name | Sequence (5′→3′) | SEQ ID NO: |
|---|---|---|---|
| pSIM6103 | JF179 | GCTGTTGTCTTCTCGATTACT | SEQ ID NO: 18 |
| pSIM6103 | JF180 | CAAGTTCATCCAAGCACCA | SEQ ID NO: 19 |
| pSIM6102 | HY4730 | GGCAAGTAGGTGTCAATGGAAAT | SEQ ID NO: 20 |
| pSIM6102 | HY4722 | CTCCACCTTCGCACTTTCTC | SEQ ID NO: 21 |
| FUS1 qPCR primer | JF183 | GTATTCACAGTCCACTGCTC | SEQ ID NO: 22 |
| FUS1 qPCR primer | JF184 | ACTCCCATTCATGACCTGTT | SEQ ID NO: 23 |
| FUS1 FAM probe | JF174-FAM | TGCCAACAGAAAGCCGCCT | SEQ ID NO: 24 |
| CHC1 qPCR | JF122 | ATGCGCTGATTGAATCTC | SEQ ID NO: 25 |
| primer | | | |
| CHC1 qPCR | JF124 | TGGCTTCCGGTTCAAATAG | SEQ ID NO: 26 |
| primer | | | |
| CHC1 HEX probe | JF150-HEX | AGCTGGAGTAGCGAGTCG | SEQ ID NO: 27 |
| pSIM6145 | JF193 | TTACATATCCGTGGAGGAGAT | SEQ ID NO: 30 |
| pSIM6145 | JF194 | GAGCGAGAGAAGGAGAACT | SEQ ID NO: 31 |
| FUS4 qPCR primer | JF240 | GGGAACTTGAAGAAATTAGGTAG | SEQ ID NO: 32 |
| FUS4 qPCR primer | JF242 | AATGTTATGGGCAAGGCC | SEQ ID NO: 33 |
| FUS4 FAM probe | JF241-FAM | GGTGCCATACCAGACTCACTAGGT | SEQ ID NO: 34 |
| Histone-mALS | RF1258 | AACCCTAACCCTAATTCC | SEQ ID NO: 35 |
| primer | | | |
| Histone-mALS | RF1259 | GAGAGGTTTTGGAGAAGG | SEQ ID NO: 36 |
| primer | | | |
| Histone-mALS | RF1260-FAM | AAGGATCTATGGCGGCCACC | SEQ ID NO: 37 |
| FAM probe | | | |
| AGP primer | RF1132 | TGCCACGTGTTTAAGGACT | SEQ ID NO: 38 |
| AGP primer | RF1133 | CCCGAAAACAAACAAATGAGAC | SEQ ID NO: 39 |
| AGP HEX probe | RF1173-HEX | AACACCACGAGGGCCCCACT | SEQ ID NO: 40 |

### EXAMPLES

The foregoing examples of the related art and limitations related therewith are intended to be illustrative and not exclusive. Other limitations of the related art will become apparent to those of skill in the art upon a reading of the specification.

### Example 1: Transforming Elite Strawberry Lines with FUS1 and/or FUS4

Plant transformation vectors were constructed using standard molecular biology techniques. Four constructs were designed, including: pSIM6102 (FIG. 1A), pSIM6103 (FIG. 1B), pSIM6145 (FIG. 1C), and pSIM6274 (FIG. 1D). pSIM6102 contains the FUS1 gene driven by its native promoter and terminator (Table 1); pSIM6103 contains the FUS1 gene excluding untranslated regions driven by the F. x ananassa M8 constitutive promoter (Table 2); pSIM6145 contains FUS1 and FUS4 driven by their native promoters and terminators (Table 3); and pSIM6274 contains the FUS4 gene driven by its native promoter and terminator (Table 4). A total of 1,140 nt upstream of the transcriptional start site was used as the native promoter and 618 nt downstream of the transcriptional stop site was used the native terminator for FUS1. A total of 1,787 nt upstream of the transcriptional start site was used as the native promoter and 696 nt downstream of the transcriptional stop site was used the native terminator for FUS4. FUS1 and FUS4 were isolated from genomic DNA from the resistant line ‘BG-4.352’ and inserted into the pENTR/D-TOPO vector (Invitrogen, Waltham, USA). To increase specificity, the sequences were initially amplified with AccuStart II Taq DNA polymerase (QuantaBio, Beverly, USA), followed by amplification with Q5 High Fidelity DNA polymerase (NEB, Newark, USA) using the first PCR product as template. Restriction enzyme digests followed by ligation with T4 DNA ligase (Promega, Madison, USA) were subsequently used to transfer the gene segments into the destination vector, which contained mutated F. vesca acetolactate synthase (mFvALS) as the selection marker. All constructs were confirmed via Sanger sequencing and were subsequently transformed into Agrobacterium tumefaciens strain GV3101.

Elite strawberry plants, including ‘Inspire’, were grown in Magenta boxes (Magenta™, Model GA-7, Millipore Sigma, St. Louis, MO) containing Fragaria ananassa rooting (FaR) medium (1/2-X M519 (PhytoTechnology Laboratories, Shawnee, KS), 15 g/L sucrose, 6 g/L agar, pH 5.7) inside a Percival growth chamber (Percival Scientific, Inc., Model CU-36L4, Perry, IA) with a 16 hour photoperiod at 23° C. Leaves of 4-6 week old plants were cut and placed on a filter paper moistened with sterile MS liquid medium (1×M519, 30 g/L sucrose, pH 5.7). The outer edges of each leaf was removed, and each leaf was cut into 3-5×3-5 mm rectangular explant sectors. Explants were placed onto pre-culture medium (TIA1: 1×M519, 30 g/L sucrose, 6 g/L agar, pH 5.7, 2 mg/L thidiazuron, 0.5 mg/L indole-3-acetic acid) with the abaxial surface (under side) in contact with the medium. Explants on pre-culture TIA1 media were placed in a Percival growth chamber at 23° C. in the dark for 7 days.

After the pre-culture period, the explants were transferred to a sterile Petri plate and transformed with various constructs using standard techniques known in the art. Explants were cultured at 24° C. in the dark for 48 hours. After the co-culture period, explants were transferred to shoot regeneration medium (TIA1) supplemented with 300 mg/L Timentin, 1 mL/L Plant Preservative Mixture (PPM), and 0.02 mg/L imazamox for plant selection. Explants were then cultured at 24° C. in the dark for 7 days, then placed under white fluorescent light (115 μmoles m-2·s-1) with a 16-hr photoperiod for an additional 21 days. Explants were then subcultured onto the same shoot regeneration medium for up to an additional 28 days.

As 4-5 mm shoot clumps formed, they were cut from the calli and transferred to Magenta boxes containing FaR medium supplemented with 300 mg/L Timentin, 1 mL/L PPM, and 0.04 mg/L imazamox for plant selection. Shoot clumps were cultured with a 16-hour photoperiod at 24° C. for 28 days. After 28 days, a single shoot per shoot clump was transferred into FaR medium supplemented with 300 mg/L Timentin and 1 mL/L PPM and cultured for an additional 28 days. Afterwards, DNA from putative transformants was screened using primers specific to the T-DNA (e.g. Table 5) to confirm its presence.

### Example 2: Fusarium Resistance Assessment of Transformed Plants

Tissue culture plants were transplanted into Sunshine Mix #1 soil (Sun Gro Horticulture, Agawam, USA) and grown at 20° C./16° C. day/night temperature in the growth chamber. After six weeks, plant roots and crown tissue were dip inoculated for 10 min with the F. oxysporum f.sp. Fragariae race 1 isolate 157 (collected in Ventura County, CA, USA in 2016) at 1×10⁶ cfu/ml concentration. The inoculum was prepared by dislodging conidia from plates with 100 ml of autoclave distilled water and a sterile cell spreader, filtering through a double layer of cheesecloth to remove hyphae, and diluting with 0.1% water agar to the final concentration. After inoculation, plants were transferred to 4-inch pots, covered with plastic domes, and grown at 28° C./20° C. day/night temperatures and 16 hour photoperiod in the greenhouse.

Plants were monitored weekly for symptoms and rated on a 0 to 10 scale (Table 6, FIG. 2). Disease scores were plotted by time and the resulting plot is called the disease progress curve. The relative area under the disease progress curve (rAUDPC) was calculated by dividing the area under the disease progress curve (AUDPC) by the AUDPC maximum potential (defined as the total number of days between the first and last rating day multiplied by 100).

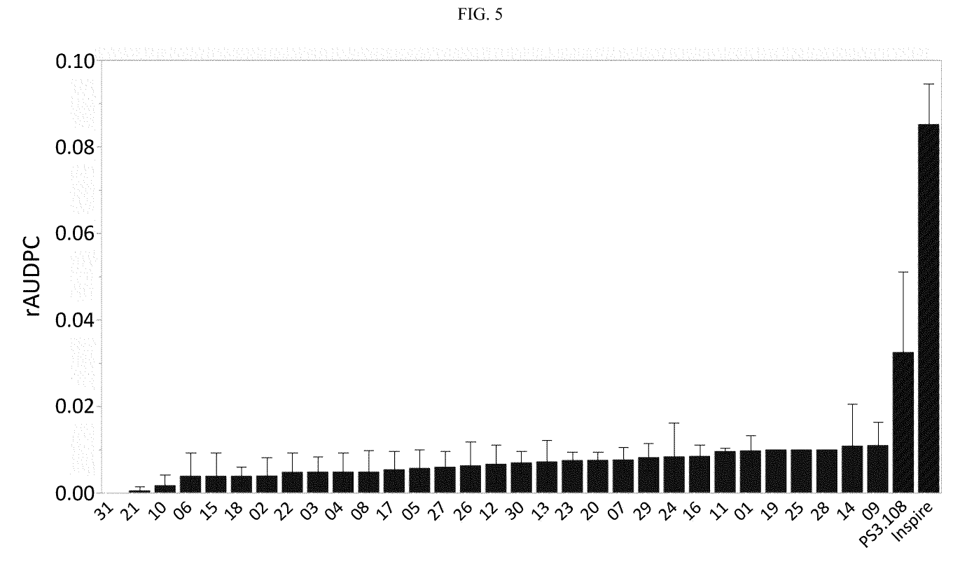

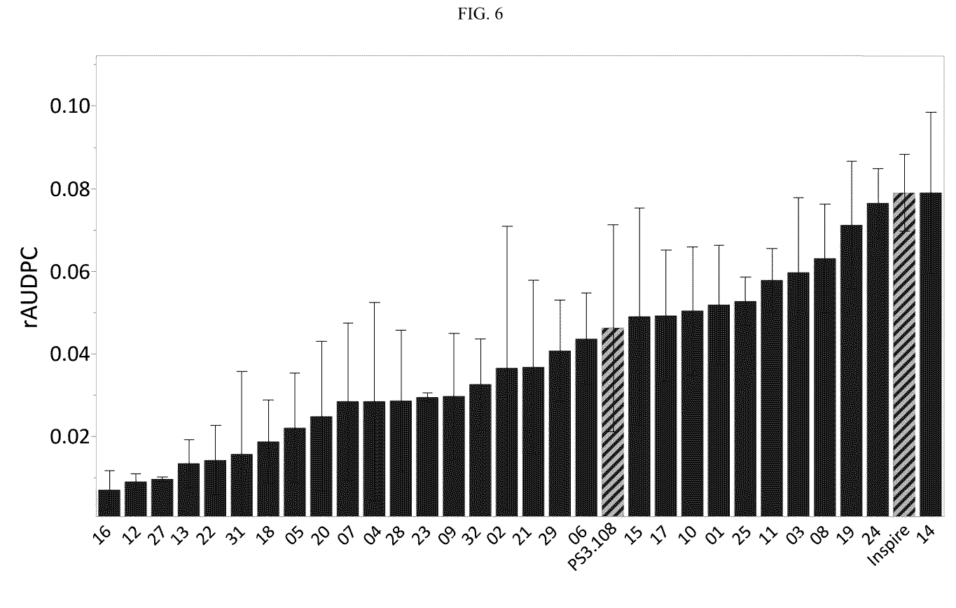

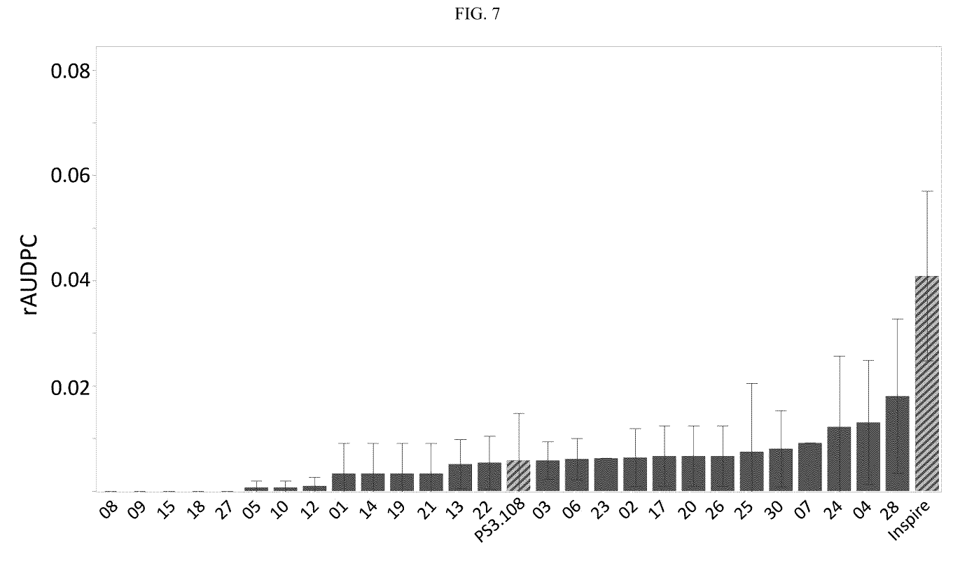

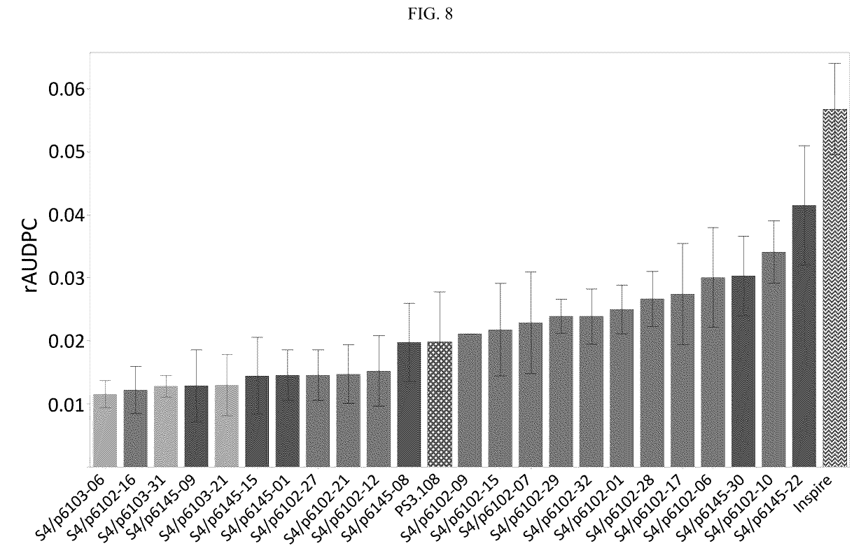

All Inspire plants transformed with pSIM6103 (i.e. constitutive expression of FUS1) resulted in significantly lower rAUDPC compared to Inspire, a susceptible variety not containing FUS1 or FUS4, and compared to PS 3.108, a resistant variety containing a single copy of FUS1 and FUS4 (FIG. 5). Inspire plants transformed with pSIM6102 (i.e. native expression of FUS1) resulted in a range of rAUDPC scores, with some lines performing better than PS 3.108 and some lines performing similarly to Inspire (FIG. 6). Out of 30 pSIM6102 lines, 18 were not significantly different from PS 3.108. Most Inspire plants transformed with pSIM6145 (i.e. native expression of both FUS1 and FUS4) displayed rAUDPC scores similar to PS 3.108, with a smaller range of rAUDPC scores (FIG. 7). To confirm results and directly compare strategies, lines from each strategy were selected and re-screened in the same disease assay (FIG. 8). pSIM6103 transformants scored similarly to the previous assay and again had significantly lower rAUDPC scores compared to both Inspire and PS 3.108. Both pSIM6102 and pSIM6145 lines displayed a range of rAUDPC scores and results largely resembled previous assays. A total of 9 pSIM6102 lines (64% of all pSIM6102 lines) and 3 pSIM6145 lines (50% of all pSIM6145 lines) were statistically similar to PS 3.108.

### Example 3: Gene Copy Number Assays of Transformed Plants

To determine the number of T-DNA insertions, droplet digital PCR (ddPCR) was used with genomic DNA, ddPCR Supermix (Bio-Rad, Hercules, USA), restriction enzyme, and the primers and probes indicated in Table 5. ADP-Glucose Pyrophosphorylase (AGP) was used as the reference. Droplets were generated with a Bio-Rad QX200 Droplet Generator (Hercules, USA) and subsequently run on a Bio-Rad C1000 Thermal Cycler (Hercules, USA) with the following program: 95° C. for 10 min followed by 44 cycles of 94° C. for 30 s, 58° C. or 56° C. for 30 s, and 60 LC for 30 s followed by a temperature of 98° C. for 10 m. The FUS1 and FvHistoneH1-mFvALS primers used an annealing temperature of 58° C. and 56° C., respectively. Afterwards, droplets were read with a Bio-Rad QX200 Droplet Reader (Hercules, USA) and data were analyzed with Bio-Rad QuantaSoft software (Hercules, USA).

Of the re-screened lines, FUS1 and FvHistoneH1-mFvALS copy number were the same for all lines which had one or two copies present (Table 8). Five lines which had higher copy number differed slightly between the two assays. PS 3.108 was confirmed to have a single copy of FUS1. Both FUS1 and FvHistoneH1-mFvALS copy number inversely correlated with the rAUDPC score (Spearman Rank correlation coefficient=−0.61 and −0.54, respectively), with more copies of FUS1 leading to lower rAUIDPC scores.

TABLE 8
Re-Screening Assay Results

| | | | FvHistoneH1- | Fof FUS1 | Fof FUS4 |
|---|---|---|---|---|---|
| | Average | FUS1 Copy | mFvALS Copy | Normalized | Normalized |
| Line | rAUDPC Score | Number | Number | Expression | Expression |
| S4/p6102-01 | 0.025 | 1 | 1 | 0.145 | NA |
| S4/p6102-06 | 0.03 | 2 | 2 | 0.644 | NA |
| S4/p6102-07 | 0.023 | 2 | 2 | 0.534 | NA |
| S4/p6102-09 | 0.021 | 2 | 2 | 0.602 | NA |
| S4/p6102-10 | 0.034 | 1 | 1 | 0.321 | NA |
| S4/p6102-12 | 0.015 | 3 | 2 | 1.805 | NA |
| S4/p6102-15 | 0.022 | 1 | 1 | 0.247 | NA |
| S4/p6102-16 | 0.012 | 7 | 5 | 1.787 | NA |
| S4/p6102-17 | 0.027 | 1 | 1 | 0.251 | NA |
| S4/p6102-21 | 0.015 | 2 | 2 | 0.470 | NA |
| S4/p6102-27 | 0.015 | 9 | 8 | 0.291 | NA |
| S4/p6102-28 | 0.027 | 2 | 2 | 0.394 | NA |
| S4/p6102-29 | 0.024 | 2 | 2 | 0.436 | NA |
| S4/p6102-32 | 0.024 | 1 | 1 | 0.380 | NA |
| S4/p6103-06 | 0.012 | 2 | 2 | 13.269 | NA |
| S4/p6103-21 | 0.013 | 3 | 2 | 19.632 | NA |
| S4/p6103-31 | 0.013 | 1 | 1 | 6.891 | NA |
| S4/p6145-01 | 0.015 | 1 | 1 | 0.325 | 2.590 |
| S4/p6145-08 | 0.02 | 5 | 5 | 0.522 | 7.734 |
| S4/p6145-09 | 0.013 | 6 | 7 | 1.395 | 13.424 |
| S4/p6145-15 | 0.014 | 3 | 3 | 0.547 | 7.157 |
| S4/p6145-22 | 0.042 | 1 | 1 | 0.083 | 1.702 |
| S4/p6145-30 | 0.03 | 1 | 1 | 0.072 | 2.256 |
| PS 3.108 | 0.02 | 1 | 0 | 0.915 | 1.336 |
| Inspire | 0.057 | 0 | 0 | NA | NA |

### Example 4: Expression Level of FUS1 and FUS4 in Transformed Plants—QPCR Assays

To quantify the expression level of FUS1 and FUS4, root tissue was collected from inoculated and non-inoculated plants at 24 hours post inoculation (HPI), and immediately flash frozen in liquid nitrogen. Roots from a single plant were designated as a replicate, and two replicates were collected per line and treatment. Total RNA was isolated using the Norgen Plant/Fungi Total RNA Purification Kit (Thorold, CA) and subsequently DNase treated with TURBO DNase (Invitrogen; Waltham, USA). DNase-treated RNA was reverse transcribed using SuperScript III first strand synthesis system (Invitrogen, Waltham, USA) followed by qPCR with AccuStart II Taq DNA polymerase (QuantaBio, Beverly, USA) with the primers and probes indicated in Table 5. Clathrin heavy chain 1 (CHC1) was used as the reference gene. FUS1 and FUS4 primers and probes were designed to specifically amplify the resistant allele. Samples were run on a Bio-Rad CFX96 Real-Time system (Hercules, USA) using the following program: 95° C. for 2 min followed by 40 cycles of 95° C. for 15 s, 58° C. for 15 s, and 72° C. for 30 s. Data were analyzed using Bio-Rad CFX Manager software (Hercules, USA). Replicates were averaged and values normalized to PS 3.108 expression. To quantify the abundance of F. oxysproum f.sp. Fragariae, previously designed primers and probes (Burkhardt et al., 2018) were used on isolated DNA.

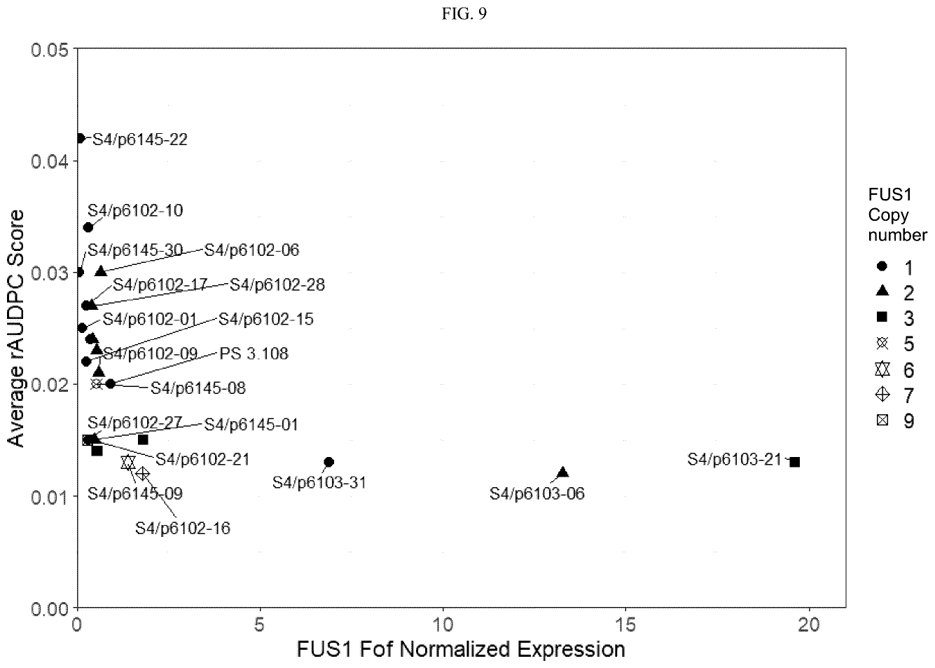

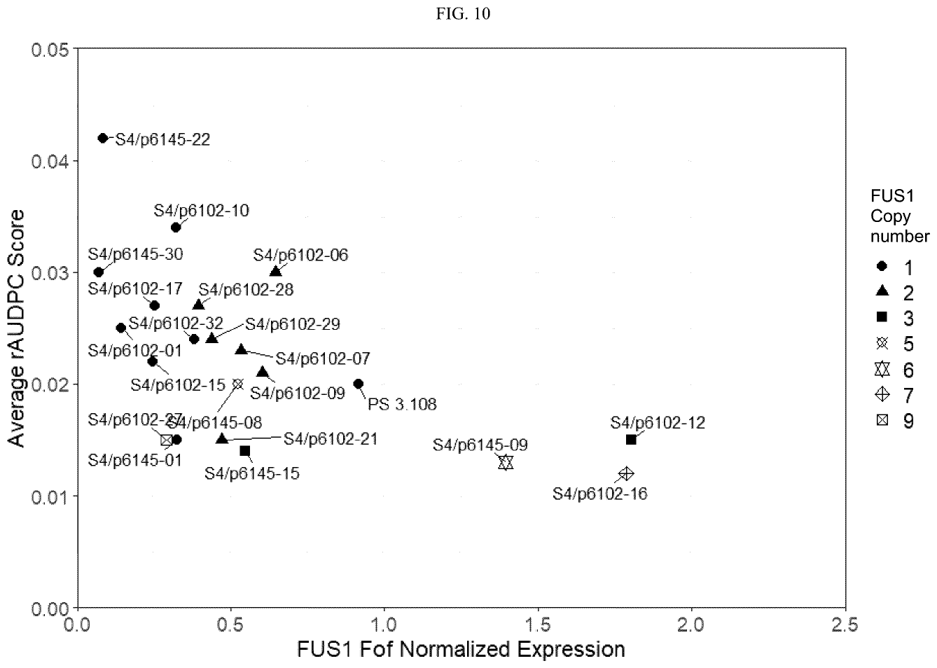

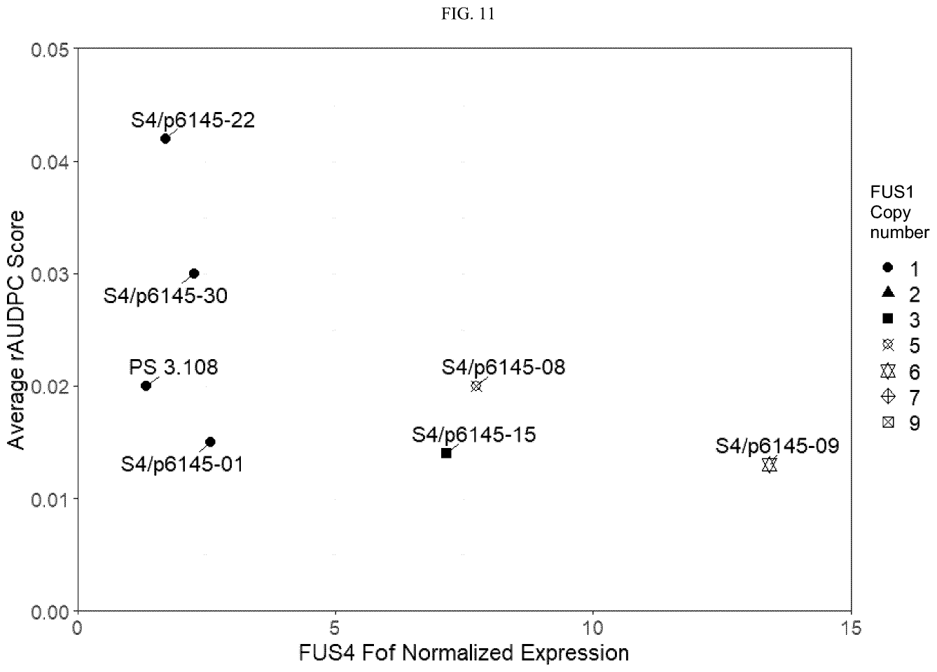

The FUS1 and FUS4 expression abundances measured on inoculated root tissue during the re-screening assay are displayed in Table 8. All pSIM6103 lines displayed high FUS1 expression (between 7-20 times the expression of PS 3.108) (Table 8, FIG. 9). Most pSIM6102 and pSIM6145 lines displayed lower expression compared to PS 3.108, while three lines (S4/p6102-12, S4/p6102-16, and S4/p6145-09) had higher expression (Table 8, FIG. 10). All single copy FUS1 lines displayed expression in the range of 0.072-0.38, while two copy FUS1 lines displayed expression in the range of 0.394-0.644. FUS1 expression was inversely correlated with rAUDPC score (Spearman Rank correlation coefficient=−0.73), with higher expression of FUS1 leading to lower rAUDPC scores. Indeed, all lines with higher FUS1 expression compared to PS 3.108 displayed better rAUDPC scores than PS 3.108. Of the pSIM6145 lines, all had the same or higher expression of FUS4 compared to PS 3.108 (Table 8, FIG. 11) and FUS4 expression was inversely correlated with rAUDPC score (Spearman Rank correlation coefficient=−0.70).

### Example 5: Marker Assisted Breeding

FUS1 and/or FUS4 primers and probes disclosed herein (for example, SEQ ID NOs: 22-24 and 32-34), or primers and probes designed based on the sequences disclosed herein, can be used for marker assisted breeding. For example, with these primers and probes, strawberry plants can be screened for the alleles and fusarium resistant lines can be identified and selected for further breeding. In one example, the selected fusarium resistant line may be crossed with an elite commercial variety. Offspring are screened for the allele(s), and progeny having the FUS1 and/or FUS4 alleles are selected for backcrossing to the elite commercial variety. This method can be repeated to generate a single locus converted elite commercial variety having fusarium resistance.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

It should be understood that the above description is only representative of illustrative embodiments and examples. For the convenience of the reader, the above description has focused on a limited number of representative examples of all possible embodiments, examples that teach the principles of the disclosure. The description has not attempted to exhaustively enumerate all possible variations or even combinations of those variations described. That alternate embodiments may not have been presented for a specific portion of the disclosure, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the disclosure. Accordingly, the disclosure is not intended to be limited to less than the scope set forth in the following claims and equivalents.

### Numbered Embodiments of the Disclosure

Notwithstanding the appended claims, the disclosure sets forth the following numbered embodiments:
1. A method for conferring Fusarium resistance in a plant, comprising at least one of: (a) introducing a nucleic acid into a plant, plant part, or plant cell, wherein said nucleic acid encodes an amino acid sequence at least 90% identical to SEQ ID NO: 2, and (b) introducing a nucleic acid into a plant, plant part, or plant cell, wherein said nucleic acid encodes an amino acid sequence at least 90% identical to SEQ ID NO: 4.
2. The method of embodiment 1, wherein the nucleic acid of (a) comprises SEQ ID NO: 1, or a sequence at least 75% identical thereto.
3. The method of embodiment 1, wherein the nucleic acid of (a) comprises SEQ ID NO: 1, or a sequence at least 85% identical thereto.
4. The method of embodiment 1, wherein the nucleic acid of (a) comprises SEQ ID NO: 28, or a sequence at least 75% identical thereto.
5. The method of embodiment 1, wherein the nucleic acid of (a) comprises SEQ ID NO: 28, or a sequence at least 85% identical thereto.
6. The method of embodiment 1, wherein, the nucleic acid of (b) comprises SEQ ID NO: 3, or a sequence at least 75% identical thereto.
7. The method of embodiment 1, wherein, the nucleic acid of (b) comprises SEQ ID NO: 3, or a sequence at least 85% identical thereto.
8. The method of embodiment 1, wherein, the nucleic acid of (b) comprises SEQ ID NO: 29, or a sequence at least 75% identical thereto.
9. The method of embodiment 1, wherein, the nucleic acid of (b) comprises SEQ ID NO: 29, or a sequence at least 85% identical thereto.
10. A Fragaria spp. plant produced by the method of any one of embodiments 1-9.
11. The Fragaria spp. plant of embodiment 10, wherein expression of said nucleotide sequence in the plant prevents Fusarium from colonizing the plant, or prevents Fusarium from affecting plant growth or marketable yield.
12. The Fragaria spp. plant of embodiment 11, wherein the Fusarium is Fusarium oxysporum.
13. A cisgenic Fragaria spp. plant produced by the method of any one of embodiments 1-12, wherein the cisgenic Fragaria spp. plant comprises a nucleic acid encoding an amino acid sequence at least 90% identical to SEQ ID NO: 2.
14. A cisgenic Fragaria spp. plant produced by the method of any one of embodiments 1-12, wherein the cisgenic Fragaria spp. plant comprises a nucleic acid encoding an amino acid sequence at least 90% identical to SEQ ID NO: 4.
15. A transgenic Fragaria spp. plant produced by the method of any one of embodiments 1-12, wherein the transgenic Fragaria spp. plant comprises a nucleic acid encoding an amino acid sequence at least 90% identical to SEQ ID NO: 2.
16. A transgenic Fragaria spp. plant produced by the method of any one of embodiments 1-12, wherein the transgenic Fragaria spp. plant comprises a nucleic acid encoding an amino acid sequence at least 90% identical to SEQ ID NO: 4.
17. The transgenic Fragaria spp. plant of embodiment 15 or 16, wherein the nucleic acid encoding an amino acid sequence at least 90% identical to SEQ ID NO: 2, and/or the nucleic acid encoding an amino acid sequence at least 90% identical to SEQ ID NO: 4 is operably linked to a constitutive promoter.
18. The transgenic Fragaria spp. plant of any one of embodiments 15-17, wherein the plant has between two and four copies of the nucleic acid encoding an amino acid sequence at least 90% identical to SEQ ID NO: 2, and/or the nucleic acid encoding an amino acid sequence at least 90% identical to SEQ ID NO: 4.
19. The transgenic Fragaria spp. plant of any one of embodiments 15-17, wherein the plant has between four and eight copies of the nucleic acid encoding an amino acid sequence at least 90% identical to SEQ ID NO: 2, and/or the nucleic acid encoding an amino acid sequence at least 90% identical to SEQ ID NO: 4.
20. The transgenic Fragaria spp. plant of any one of embodiments 10-19, wherein the transgenic Fragaria spp. plant has between 1.1-3 fold increase in expression of the nucleic acid encoding an amino acid sequence at least 90% identical to SEQ ID NO: 2 compared to a non-transgenic Fragaria spp. plant.
21. The transgenic Fragaria spp. plant of any one of embodiments 10-19, wherein the transgenic Fragaria spp. plant has between 1.1-3 fold increase in expression of the nucleic acid encoding an amino acid sequence at least 90% identical to SEQ ID NO: 4 compared to a non-transgenic Fragaria spp. plant.
22. The transgenic Fragaria spp. plant of embodiment 17, wherein the transgenic Fragaria spp. plant has between a 3- and 5-fold increase in expression of the nucleic acid encoding an amino acid sequence at least 90% identical to SEQ ID NO: 4 and/or the nucleic acid encoding an amino acid sequence at least 90% identical to SEQ ID NO: 2 compared to a non-transgenic Fragaria spp. plant.
23. The transgenic Fragaria spp. plant of embodiment 17, wherein the transgenic Fragaria spp. plant has between a 5- and 10-fold increase in expression of the nucleic acid encoding an amino acid sequence at least 90% identical to SEQ ID NO: 4 and/or the nucleic acid encoding an amino acid sequence at least 90% identical to SEQ ID NO: 2 compared to a non-transgenic Fragaria spp. plant.
24. The transgenic Fragaria spp. plant of embodiment 17, wherein the transgenic Fragaria spp. plant has between a 10- and 15-fold increase in expression of the nucleic acid encoding an amino acid sequence at least 90% identical to SEQ ID NO: 4 and/or the nucleic acid encoding an amino acid sequence at least 90% identical to SEQ ID NO: 2 compared to a non-transgenic Fragaria spp. plant.
25. The transgenic Fragaria spp. plant of embodiment 17, wherein the transgenic Fragaria spp. plant has between a 15- and 20-fold increase in expression of the nucleic acid encoding an amino acid sequence at least 90% identical to SEQ ID NO: 4 and/or the nucleic acid encoding an amino acid sequence at least 90% identical to SEQ ID NO: 2 compared to a non-transgenic Fragaria spp. plant.
26. The Fragaria spp. plant of any one of embodiments 10-12, the cisgenic Fragaria spp. plant of any one of embodiments 13-14, or the transgenic Fragaria spp. plant of any one of embodiments 15-25, wherein the plant has a Fusarium wilt resistance rAUDPC score of less than 0.04.
27. The Fragaria spp. plant of any one of embodiments 10-12, the cisgenic Fragaria spp. plant of any one of embodiments 13-14, or the transgenic Fragaria spp. plant of any one of embodiments 15-25, wherein the plant has a Fusarium wilt resistance rAUDPC score of less than 0.03.
28. The Fragaria spp. plant of any one of embodiments 10-12, the cisgenic Fragaria spp. plant of any one of embodiments 13-14, or the transgenic Fragaria spp. plant of any one of embodiments 15-25, wherein the plant has a Fusarium wilt resistance rAUDPC score of less than 0.02.
29. The Fragaria spp. plant of any one of embodiments 10-12, the cisgenic Fragaria spp. plant of any one of embodiments 13-14, or the transgenic Fragaria spp. plant of any one of embodiments 15-25, wherein the plant has a Fusarium wilt resistance rAUDPC score of less than 0.01.
30. The Fragaria spp. plant of any one of embodiments 10-12, the cisgenic Fragaria spp. plant of any one of embodiments 13-14, or the transgenic Fragaria spp. plant of any one of embodiments 15-25, wherein the plant has a Fusarium wilt resistance rAUDPC score of between 0.01 and 0.02.
31. A method for producing a Fragaria spp. plant having resistance or tolerance to Fusarium wilt, comprising: applying a plant breeding technique to the Fragaria spp. plant of any one of embodiments 10-30.
32. The method of embodiment 31, wherein the plant breeding technique is selected from the group consisting of recurrent selection, mass selection, hybridization, open-pollination, backcrossing, pedigree breeding, mutation breeding, and marker enhanced selection.
33. A Fragaria spp. plant having resistance or tolerance to Fusarium wilt produced by the method of embodiment 31 or 32.
34. A method of producing a Fragaria spp. plant comprising a Fusarium wilt resistance allele, comprising:
   screening a Fragaria spp. plant for the presence of a Fusarium wilt resistance allele designated FUS1, wherein said FUS1 allele encodes a protein comprising SEQ ID NO: 2 or a sequence at least 90% identical thereto, and wherein the protein is a TIR-NB-ARC-LRR protein that comprises a C-terminal Jelly-roll/Ig-like Domain, and selecting a Fragaria spp. plant comprising said FUS1 allele;
   crossing the selected Fragaria spp. plant comprising said FUS1 allele with a second Fragaria spp. plant to produce progeny Fragaria spp. plants;
   screening said progeny Fragaria spp. plants for the presence of said FUS1 allele; and
   selecting a progeny Fragaria spp. plant comprising at least one copy of said FUS1 allele.
35. The method of embodiment 34, wherein the FUS1 allele comprises a sequence at least 80% identical to SEQ ID NO: 1.
36. The method of embodiment 34, wherein the FUS1 allele comprises a sequence at least 90% identical to SEQ ID NO: 1.
37. The method of embodiment 34, wherein the FUS1 allele comprises a sequence at least 80% identical to SEQ ID NO: 28.
38. The method of embodiment 34, wherein the FUS1 allele comprises a sequence at least 90% identical to SEQ ID NO: 28.
39. The method of any one of embodiments 34-38, wherein the FUS1 allele encoding a protein having at least 90% identity SEQ ID NO: 2 comprises a C-JID sequence having a serine at a position corresponding to 1,046 of SEQ ID NO: 2; a phenylalanine at a position corresponding to 1,060 of SEQ ID NO: 2; a serine at a position corresponding to 1,063 of SEQ ID NO: 2; a leucine at a position corresponding to 1,065 of SEQ ID NO: 2; an asparagine at a position corresponding to 1,069 of SEQ ID NO: 2; a tyrosine at a position corresponding to 1,072 of SEQ ID NO: 2; a lysine at a position corresponding to 1,073 of SEQ ID NO: 2; a phenylalanine at a position corresponding to 1,083 of SEQ ID NO: 2; a valine at a position corresponding to 1,121 of SEQ ID NO: 2; a histidine at a position corresponding to 1,122 of SEQ ID NO: 2; a cysteine at a position corresponding to 1,123 of SEQ ID NO: 2; a proline at a position corresponding to 1,126 of SEQ ID NO: 2; and an arginine at a position corresponding to 1,130 of SEQ ID NO: 2.
39.1 The method of any one of embodiments 34-39, wherein the FUS1 allele encoding a protein having at least 90% identity to SEQ ID NO: 2 further comprises an alanine at a position corresponding to 1,078 of SEQ ID NO:2; an isoleucine at a position corresponding to 1,079 of SEQ ID NO:2; a threonine at a position corresponding to 1,120 of SEQ ID NO: 2; and a leucine at a position corresponding to 1,153 of SEQ ID NO: 2
40. The method of any one of embodiments 34-39, wherein the screening a Fragaria spp. plant for the presence of said FUS1 allele comprises marker-assisted selection.
41. The method of embodiment 40, wherein the marker-assisted selection comprises a DNA marker selected from the group consisting of SEQ ID NO: 1, cDNA sequences thereof, fragments of at least 18 consecutive nucleotides thereof, and complementary sequences thereof.
42. The method of embodiment 41, wherein the marker-assisted selection comprises a DNA marker selected from the group consisting of SEQ ID NO: 28, cDNA sequences thereof, fragments of at least 18 consecutive nucleotides thereof, and complementary sequences thereof.
43. A method of producing a Fragaria spp. plant comprising a Fusarium wilt resistance allele, comprising:
   screening a Fragaria spp. plant for the presence of a Fusarium wilt resistance allele designated FUS4, wherein said FUS4 allele encodes a protein comprising SEQ ID NO: 4, or a sequence at least 90% identical thereto, and wherein the protein encoded by said allele is a leucine rich receptor like kinase, and selecting a Fragaria spp. plant comprising said FUS4 allele;
   crossing the selected Fragaria spp. plant comprising said FUS4 allele with a second Fragaria spp. plant to produce progeny Fragaria spp. plants;
   screening said progeny Fragaria spp. plants for the presence of said FUS4 allele; and selecting a progeny Fragaria spp. plant comprising at least one copy of said FUS4 allele.
44. The method of embodiment 43, wherein the FUS4 allele comprises a sequence at least 80% identical to SEQ ID NO: 3.
45. The method of embodiment 43, wherein the FUS4 allele comprises a sequence at least 90% identical to SEQ ID NO: 3.
46. The method of embodiment 43, wherein the FUS4 allele comprises a sequence at least 80% identical to SEQ ID NO: 29.
47. The method of embodiment 43, wherein the FUS4 allele comprises a sequence at least 90% identical to SEQ ID NO: 29.
48. The method of any one of embodiments 43-47, wherein the FUS4 allele encoding a protein having at least 90% identity SEQ ID NO: 4 comprises a phenylalanine at a position corresponding to 256 of SEQ ID NO: 4; a tryptophan at a position corresponding to 444 of SEQ ID NO: 4; a glycine at a position corresponding to 509 of SEQ ID NO: 4; and a threonine at a position corresponding to 545 of SEQ ID NO: 4.
49. The method of any one of embodiments 43-48, wherein the screening a Fragaria spp. plant for the presence of a Fusarium wilt resistance gene comprises marker-assisted selection.
50. The method of embodiment 49, wherein the marker-assisted selection comprises a DNA marker selected from the group consisting of SEQ ID NO: 3, cDNA sequences thereof, fragments of at least 18 consecutive nucleotides thereof, and complementary sequences thereof.
51. The method of embodiment 49, wherein the marker-assisted selection comprises a DNA marker selected from the group consisting of SEQ ID NO: 29, cDNA sequences thereof, fragments of at least 18 consecutive nucleotides thereof, and complementary sequences thereof.
52. The method of any one of embodiments 34-51, wherein the screening a Fragaria spp. plant for the presence of a Fusarium wilt resistance allele comprises a DNA amplification-based method.
53. The method of any one of embodiments 34-52, wherein the screening a Fragaria spp. plant for the presence of a Fusarium wilt resistance allele comprises restriction fragment length polymorphism, amplified fragment length polymorphism, random amplified polymorphic DNA, cleaved amplified polymorphic sequences, simple sequence repeat length polymorphism, single strand conformational polymorphism, heteroduplex analysis, single nucleotide polymorphism, expressed sequence tags, or sequence tagged sites.
54. The method of any one of embodiments 34-53, wherein the progeny Fragaria spp. plant comprising at least one copy of said Fusarium wilt resistance allele is resistant to Fusarium oxysporum f. sp. Fragariae.
55. The method of any one of embodiments 34-54, wherein the Fragaria spp. plant for breeding and/or the second Fragaria spp. plant are Fragaria x ananassa.
56. The method of any one of embodiments 34-55, further comprising: asexual propagation, outcrossing, or backcrossing the selected progeny Fragaria spp. plant.
57. A molecular marker for a Fusarium wilt resistance allele, comprising: at least one sequence selected from the group consisting of SEQ ID NO: 1, cDNA sequences thereof, fragments of at least 18 consecutive nucleotides thereof, and complementary sequences thereof.
58. A method for distinguishing a Fragaria spp. plant having at least one Fusarium wilt resistance allele, comprising: using the molecular marker of embodiment 57.
59. The method of embodiment 58, wherein the molecular marker is selected from SEQ ID NOs: 22-24, and combinations thereof.
60. A molecular marker for distinguishing a plant having at least one Fusarium wilt resistance allele, comprising: at least one sequence selected from the group consisting of SEQ ID NO: 3, cDNA sequences thereof, fragments of at least 18 consecutive nucleotides thereof, and complementary sequences thereof.
61. A method for distinguishing a Fragaria spp. plant having at least one Fusarium wilt resistance allele, comprising: using the molecular marker of embodiment 60.
62. The method of embodiment 61, wherein the molecular marker is selected from SEQ ID NOs: 32-34, and combinations thereof.
63. A recombinant nucleic acid molecule comprising a nucleotide sequence encoding SEQ ID NO: 2, or a sequence at least 90% identical thereto.
64. The recombinant nucleic acid molecule of embodiment 63, wherein the nucleotide sequence encoding SEQ ID NO: 2 is SEQ ID NO: 1, or a sequence at least 75% identical thereto.
65. The recombinant nucleic acid molecule of embodiment 63, wherein the nucleotide sequence encoding SEQ ID NO: 2 is SEQ ID NO: 28, or a sequence at least 75% identical thereto.
66. The recombinant nucleic acid molecule of any one of embodiments 63-65, wherein the nucleotide sequence encoding SEQ ID NO: 2, or a sequence at least 90% identical thereto is operably linked to a tissue specific promoter.
67. The recombinant nucleic acid molecule of any one of embodiments 63-65, wherein the nucleotide sequence encoding SEQ ID NO: 2, or a sequence at least 90% identical thereto is operably linked to a constitutive promoter.
68. A recombinant nucleic acid molecule comprising a nucleotide sequence encoding SEQ ID NO: 4, or a sequence at least 90% identical thereto.
69. The recombinant nucleic acid molecule of embodiment 68, wherein the nucleotide sequence encoding SEQ ID NO: 4 is SEQ ID NO: 3, or a sequence at least 75% identical thereto.
70. The recombinant nucleic acid molecule of embodiment 68, wherein the nucleotide sequence encoding SEQ ID NO: 4 is SEQ ID NO: 29, or a sequence at least 75% identical thereto.
71. The recombinant nucleic acid molecule of any one of embodiments 68-70, wherein the nucleotide sequence encoding SEQ ID NO: 4, or a sequence at least 90% identical thereto is operably linked to a tissue specific promoter.
72. The recombinant nucleic acid molecule of any one of embodiments 68-70, wherein the nucleotide sequence encoding SEQ ID NO: 4, or a sequence at least 90% identical thereto is operably linked to a constitutive promoter.
73. A vector comprising the recombinant nucleic acid molecule of any one of embodiments 63-72.
74. A method for identifying a plant, plant part, or plant cell comprising a FUS1 allele, which confers resistance to Fusarium wilt when present in a strawberry plant, wherein said FUS1 allele encodes a TIR-NB-ARC-LRR protein that comprises in its amino acid sequence a C-terminal Jelly-roll/Ig-like Domain, the method comprising: determining the presence of a genomic nucleotide sequence or a resistance-conferring part thereof in the genome of a plant, plant part, or plant cell, wherein said nucleotide sequence encodes a protein having at least 90% sequence similarity to SEQ ID NO: 2.
75. The method of embodiment 74, wherein the nucleotide sequence encodes a protein having at least 95% sequence similarity to SEQ ID NO: 2.
76. The method of embodiment 74, wherein the nucleotide sequence comprises SEQ ID NO: 1 or a sequence at least 75% identical thereto.
77. The method of embodiment 74, wherein the nucleotide sequence comprises SEQ ID NO: 1 or a sequence at least 85% identical thereto.
78. The method of embodiment 74, wherein the nucleotide sequence comprises SEQ ID NO: 28 or a sequence at least 75% identical thereto.
79. The method of embodiment 74, wherein the nucleotide sequence comprises SEQ ID NO: 28 or a sequence at least 85% identical thereto.
80. The method of any one of embodiments 74-79, wherein determining the presence of a genomic nucleotide sequence or a resistance-conferring part thereof comprises biomolecular characterization.
81. A method for identifying a plant, plant part, or plant cell comprising a FUS4 allele, which confers resistance to Fusarium wilt when present in a strawberry plant, wherein said FUS4 allele encodes a leucine rich receptor like kinase, said method comprising: determining the presence of genomic nucleotide sequence or a resistance-conferring part thereof in the genome of a plant, plant part, or plant cell, wherein said nucleotide sequence encodes a protein having at least 90% sequence similarity to SEQ ID NO: 4.
82. The method of embodiment 81, wherein the nucleotide sequence encodes a protein having at least 95% sequence similarity to SEQ ID NO: 4.
83. The method of embodiment 81, wherein the nucleotide sequence comprises SEQ ID NO: 3 or a sequence at least 75% identical thereto.
84. The method of embodiment 81, wherein the nucleotide sequence comprises SEQ ID NO: 3 or a sequence at least 85% identical thereto.
85. The method of embodiment 81, wherein the nucleotide sequence comprises SEQ ID NO: 29 or a sequence at least 75% identical thereto.
86. The method of embodiment 81, wherein the nucleotide sequence comprises SEQ ID NO: 29 or a sequence at least 85% identical thereto.
87. The method of any one of embodiments 81-86, wherein determining the presence of a genomic nucleotide sequence or a resistance-conferring part thereof comprises biomolecular characterization.
88. A molecular marker for a Fusarium wilt resistance allele, comprising: at least one sequence selected from the group consisting of SEQ ID NO: 28, cDNA sequences thereof, fragments of at least 18 consecutive nucleotides thereof, and complementary sequences thereof.
89. A method for distinguishing a Fragaria spp. plant having at least one Fusarium wilt resistance allele, comprising: using the molecular marker of embodiment 88.
90. A molecular marker for distinguishing a plant having at least one Fusarium wilt resistance allele, comprising: at least one sequence selected from the group consisting of SEQ ID NO: 29, cDNA sequences thereof, fragments of at least 18 consecutive nucleotides thereof, and complementary sequences thereof.
91. A method for distinguishing a Fragaria spp. plant having at least one Fusarium wilt resistance allele, comprising: using the molecular marker of embodiment 90.
92. A genetically engineered Fragaria spp. plant or plant part comprising a Fusarium wilt resistance allele designated FUS4, wherein said FUS4 allele encodes a protein comprising SEQ ID NO: 4, or a sequence at least 90% identical thereto, wherein the protein encoded by said allele is a leucine rich receptor like kinase, and wherein said genetically engineered Fragaria spp. plant or plant part has a Fusarium wilt resistance rAUDPC score of less than 0.04.
93. A genetically engineered Fragaria spp. plant or plant part comprising a Fusarium wilt resistance allele designated FUS1, wherein said FUS1 allele encodes a protein comprising SEQ ID NO: 2, or a sequence at least 90% identical thereto, wherein said FUS1 allele encodes a TIR-NB-ARC-LRR protein that comprises in its amino acid sequence a C-terminal Jelly-roll/Ig-like Domain, and wherein said genetically engineered Fragaria spp. plant or plant part has an average Fusarium wilt resistance rAUDPC score of less than 0.04.
94. The genetically engineered Fragaria spp. plant or plant part of embodiment 92 or 93, wherein the genetically engineered Fragaria spp. plant or plant part has an average Fusarium wilt resistance rAUDPC score of less than 0.03.
95. The genetically engineered Fragaria spp. plant or plant part of embodiment 92 or 93, wherein the genetically engineered Fragaria spp. plant or plant part has an average Fusarium wilt resistance rAUDPC score of less than 0.02.
96. The genetically engineered Fragaria spp. plant or plant part of embodiment 92 or 93, wherein the genetically engineered Fragaria spp. plant or plant part has an average Fusarium wilt resistance rAUDPC score of less than 0.01.
97. The genetically engineered Fragaria spp. plant or plant part of embodiment 92 or 93, wherein the genetically engineered Fragaria spp. plant or plant part has an average Fusarium wilt resistance rAUDPC score of between 0.01 and 0.02.
98. The genetically engineered Fragaria spp. plant or plant part of embodiment 92, wherein the FUS4 allele is driven by its native promoter.
99. The genetically engineered Fragaria spp. plant or plant part of any one of embodiments 92 or 94-97, wherein the FUS4 allele is driven by a constitutive promoter.
100. The genetically engineered Fragaria spp. plant or plant part of embodiment 99, wherein the plant or plant part has increased FUS4 expression compared to a non-transgenic Fragaria spp. plant or plant part.
101. The genetically engineered Fragaria spp. plant or plant part of embodiment 100, wherein the expression of FUS4 is between 1.1 and 20-fold greater than a non-transgenic Fragaria spp. plant or plant part.
102. The genetically engineered Fragaria spp. plant or plant part of embodiment 101, wherein the expression of FUS4 is between 1.1 and 10-fold greater than a non-transgenic Fragaria spp. plant or plant part.
103. The genetically engineered Fragaria spp. plant or plant part of embodiment 101, wherein the expression of FUS4 is between 10 and 20-fold greater than a non-transgenic Fragaria spp. plant or plant part.
104. The genetically engineered Fragaria spp. plant or plant part of embodiment 93, wherein the FUS1 allele is driven by its native promoter.
105. The genetically engineered Fragaria spp. plant or plant part of embodiment 93, wherein the FUS1 allele is driven by a constitutive promoter.
106. The genetically engineered Fragaria spp. plant or plant part of embodiment 105, wherein the plant or plant part has increased FUS1 expression compared to a non-transgenic Fragaria spp. plant or plant part.
107. The genetically engineered Fragaria spp. plant or plant part of embodiment 106, wherein the expression of FUS1 is between 1.1 and 20-fold greater than a non-transgenic Fragaria spp. plant or plant part.
108. The genetically engineered Fragaria spp. plant or plant part of embodiment 107, wherein the expression of FUS1 is between 1.1 and 10-fold greater than a non-transgenic Fragaria spp. plant or plant part.
109. The genetically engineered Fragaria spp. plant or plant part of embodiment 107, wherein the expression of FUS1 is between 10 and 20-fold greater than a non-transgenic Fragaria spp. plant or plant part.

What is claimed is:

1. A genetically engineered Fragaria spp. plant or plant part comprising a Fusarium wilt resistance allele designated FUS1, wherein said FUS1 allele encodes a protein comprising SEQ ID NO: 2, or a sequence at least 90% identical thereto, wherein said FUS1 allele encodes a TIR-NB-ARC-LRR protein that comprises in its amino acid sequence a C-terminal Jelly-roll/Ig-like Domain, and wherein said genetically engineered Fragaria spp. plant or plant part has an average Fusarium wilt resistance rAUDPC score of less than 0.04.

2. The genetically engineered Fragaria spp. plant or plant part of claim 1, wherein the FUS1 allele encodes a protein at least 95% identical to SEQ ID NO: 2.

3. The genetically engineered Fragaria spp. plant or plant part of claim 1, wherein the FUS1 allele comprises:
SEQ ID NO: 1, or a sequence at least 75% identical thereto, or
SEQ ID NO: 28, or a sequence at least 75% identical thereto.

4. The genetically engineered Fragaria spp. plant or plant part of claim 1, wherein the genetically engineered Fragaria spp. plant or plant part has an average Fusarium wilt resistance rAUDPC score of less than 0.03.

5. The genetically engineered Fragaria spp. plant or plant part of claim 1, wherein the genetically engineered Fragaria spp. plant or plant part has an average Fusarium wilt resistance rAUDPC score of less than 0.02.

6. The genetically engineered Fragaria spp. plant or plant part of claim 1, wherein the genetically engineered Fragaria spp. plant or plant part has an average Fusarium wilt resistance rAUDPC score of less than 0.01.

7. The genetically engineered Fragaria spp. plant or plant part of claim 1, wherein the genetically engineered Fragaria spp. plant or plant part has an average Fusarium wilt resistance rAUDPC score of between 0.01 and 0.02.

8. The genetically engineered Fragaria spp. plant or plant part of claim 1, wherein the FUS1 allele is driven by its native promoter.

9. The genetically engineered Fragaria spp. plant or plant part of claim 1, wherein the FUS1 allele is driven by a constitutive promoter.

10. The genetically engineered Fragaria spp. plant or plant part of claim 7, wherein the plant or plant part has increased FUS1 expression compared to a non-transgenic Fragaria spp. plant or plant part.

11. The genetically engineered Fragaria spp. plant or plant part of claim 8, wherein the expression of FUS1 is between 1.1 and 20-fold greater than a non-transgenic Fragaria spp. plant or plant part.

12. The genetically engineered Fragaria spp. plant or plant part of claim 9, wherein the expression of FUS1 is between 1.1 and 10-fold greater than a non-transgenic Fragaria spp. plant or plant part.

13. The genetically engineered Fragaria spp. plant or plant part of claim 9, wherein the expression of FUS1 is between 10 and 20-fold greater than a non-transgenic Fragaria spp. plant or plant part.

14. The genetically engineered Fragaria spp. plant or plant part of claim 1, further comprising a Fusarium wilt resistance allele designated FUS4, wherein said FUS4 allele encodes a protein comprising SEQ ID NO: 4, or a sequence at least 90% identical thereto, wherein the protein encoded by said allele is a leucine rich receptor like kinase.

15. A method for conferring Fusarium resistance in a plant, comprising at least one of: (a) introducing a nucleic acid into a plant, plant part, or plant cell, wherein said nucleic acid encodes an amino acid sequence at least 90% identical to SEQ ID NO: 2, and (b) introducing a nucleic acid into a plant, plant part, or plant cell, wherein said nucleic acid encodes an amino acid sequence at least 90% identical to SEQ ID NO: 4.

16. The method of claim 15, wherein the nucleic acid of (a) comprises SEQ ID NO: 1, or a sequence at least 75% identical thereto.

17. The method of claim 15, wherein the nucleic acid of (a) comprises SEQ ID NO: 1, or a sequence at least 85% identical thereto.

18. The method of claim 15, wherein the nucleic acid of (a) comprises SEQ ID NO: 28, or a sequence at least 75% identical thereto.

19. The method of claim 15, wherein the nucleic acid of (a) comprises SEQ ID NO: 28, or a sequence at least 85% identical thereto.

20. A molecular marker for a Fusarium wilt resistance allele, comprising: at least one sequence selected from the group consisting of SEQ ID NO: 1, cDNA sequences thereof, fragments of at least 18 consecutive nucleotides thereof, and complementary sequences thereof.
